(19) United States
(12) Patent Application Publication
(10) Pub. No.: US 2019/0298824 A1
(43) Pub. Date: Oct. 3, 2019

(54) ALBUMIN-BINDING IMMUNOMODULATORY COMPOSITIONS AND METHODS OF USE THEREOF

(71) Applicant: THE UNITED STATES OF AMERICA, as represented by the Secretary, Department of Health and Human Serv, Bethesda, MD (US)

(72) Inventors: Xiaoyuan Chen, Potomac, MS (US); Guizhi Zhu, Richmond, VA (US)

(73) Assignee: THE UNITED STATES OF AMERICA, as represented by the Secretary, Department of Health and Human Serv, Bethesda, MD (US)

(21) Appl. No.: 16/098,115

(22) PCT Filed: May 4, 2017
(86) PCT No.: PCT/US2017/031098

§ 371 Date: Oct. 31, 2018

Related U.S. Application Data
(60) Provisional application No. 62/331,890, filed on May 4, 2016.

(51) Int. Cl. A61K 39/39; A61K 49/00; A61K 47/54; A61K 47/65

(52) U.S. Cl. CPC A61K 39/39; A61K 49/0052; A61K 49/0021; A61K 47/65; A61K 47/545; A61K 47/542; A61K 47/549

(57) ABSTRACT

The present disclosure concerns chemical conjugates having structures satisfying Formula I and methods of making and using these chemical conjugates.

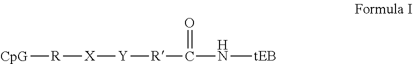

Also described are immunomodulatory compositions comprising the chemical conjugates and methods of using the chemical conjugates and/or immunomodulatory compositions thereof to induce an immune response in a subject.

### CROSS REFERENCE TO RELATED APPLICATION

This application is a § 371 U.S. national stage of International Application No. PCT/US2017/031098, filed May 4, 2017, which was published in English under PCT Article 21(2), which claims the benefit of U.S. Provisional Application No. 62/331,890, filed May 4, 2016, which is incorporated by reference herein.

### FIELD OF THE DISCLOSURE

This relates to the field of immunotherapy, specifically to chemical conjugates that are immunostimulatory, and can be used to induce an immune response.

### BACKGROUND

Immunotherapy harnesses the host immune system for disease therapy, and is applicable to a wide range of diseases, including tumors, infectious diseases, and autoimmune diseases such as Type I diabetes (Rosenberg et al., 2004, Nature Medicine 10(9):909-915; Sharma and Allison, 2015, Science 348(6230):56-61). Subunit vaccines represent an alternative class of immunotherapeutics that activate antigen presenting cells (APCs) and modulate T cell priming, in order to educate the immune system and tune its immune responses to tumors, pathogens, or self-tissue in the case of autoimmune diseases. For optimal immunotherapy outcome, it is critical to efficiently deliver and sustainably retain subunit vaccines into the secondary lymphoid organs, where versatile immune cells reside and immune responses are orchestrated. This need for vaccine delivery into secondary lymphoid organs is further strengthened by the systemic toxicity induced by many unconjugated vaccine components, of which one prominent example is nucleic acid adjuvants, including immunostimulatory CpG oligodeoxynucleotides (ODN) (Klinman, 2004, Nature reviews. Immunology 4(4):249-258; Hartmann et al., 1999, Proceedings of the National Academy of Sciences of the United States of America 96(16):9305-9310). CpG ODNs are a class of unmethylated oligonucleotides containing cytosine-guanine motifs derived from pathogen-associated molecular patterns (PAMPs). CpG ODNs recognize Toll-like receptor 9 (TLR9) and stimulate innate immune responses in many immune cells, making CpG one of the most potent molecular adjuvants. However, clinical translation of CpG has been thwarted by its toxicity, which is mainly associated with systemic inflammation induced by CpG disseminated into the systemic circulation (Bourquin C (2008) Targeting CpG oligonucleotides to the lymph node by nanoparticles elicits efficient antitumoral immunity. Journal of immunology 181:2990-2998).

Emulsion-based formulation, such as Incomplete Freud's Adjuvant (IFA), has been implemented for vaccine delivery by encapsulating vaccines inside emulsion and slowly release vaccines after vaccination for a prolonged efficacy. Additionally, nanoparticulate vaccines have been studied for vaccine delivery into secondary lymphoid organs such as lymph nodes (LNs) (Irvine et al., 2015, Synthetic Nanoparticles for Vaccines and Immunotherapy. Chemical Reviews:2018197663; Reddy, 2007, Nature Biotechnol. 25:1159-1164; Hubbell et al., 2009, Materials engineering for immunomodulation. Nature 462:449-460; Lynn et al., 2015, Nature Biotechnology 33(11): 1201-1210; Kim et al., 2015, Nature Biotechnology 33(1):64-72; Liu et al, 2012, Nano Letters 12(8):4254-4259; Beaudette et al., 2009, Molecular Pharmaceutics 6(4): 1160-1169; Nishikawa et al., 2011, Biomaterials 32(2):488-494; Moon et al., 2012, Adv. Mater. 24:3724-3746; Li et al., 2011, ACS nano 5(11):8783-8789), which are distributed throughout the body and filter lymph before lymph is conducted into blood circulation. Nanoparticulate vaccines that mimic nanoscale pathogens can be efficiently drained by lymphatic system into secondary lymphoid organs. Further, the relatively large sizes and the “foreign” nature of nanoparticulate vaccines prevent them from being rapidly flushed away from LNs and allow them to be recognized and taken up by APCs, thereby offering a time window during which nanoparticulate vaccines can have enhanced interaction with residing immune cells in LNs for immunoregulation. LN injection, though invasive to some extent and require ultrasound imaging for guidance in clinic, has proved to efficiently administer vaccines into LNs for potent regulation of LN microenvironment (Kheirolomoom et al., 2015, Journal of controlled release: official journal of the Controlled Release Society 220(Pt A):253-264).

Alternatively, molecular vaccines are attractive for their defined chemical and pharmacological characteristics. However, molecular vaccines can often be rapidly flushed into the systemic circulation, resulting in both systemic toxicity and rapid clearance from the body. To address this dilemma, molecular vaccines that can leverage endogenous nanocarriers are highly desired for efficient delivery and prolonged retention in secondary lymphoid organs.

Albumin nanoparticles account for approximately 50% of proteins in lymph as well as blood, and it is also ubiquitous in extravascular fluid (Kratz, 2008, Journal of Controlled Release 132(3):171-183). Albumin is extremely stable, with the half-life of human serum albumin, for instance, as long as 20 days. Albumin serves as a natural carrier for molecules with low hydrophilicity, cations, fatty acids, and drugs. Indeed, albumin has been long sought after as carriers of theranostic compounds for drug delivery and imaging in clinic. For example, paclitaxel, an anticancer drug, is an exemplary drugs that bind albumin, and the resulting complex, Abraxane, improves the pharmacological behaviors and overall therapeutic outcome of paclitaxel. The exploration of albumin as vaccine carriers in the lymphatic system has recently emerged (Liu et al., 2014, Nature 507:519-522). As interstitial fluid is drained through the LNs, immune cells and proteins (e.g., albumin) in the lymph are temporarily retained in LNs, which, combined with the relatively slow lymphatic draining, offers a time window for albumin-binding vaccines to be retained in LN microenvironment and manipulate the immune system via predesignated interaction with immune cells residing in LNs.

### SUMMARY OF THE DISCLOSURE

Disclosed herein are embodiments of chemical conjugates having structures satisfying Formula I

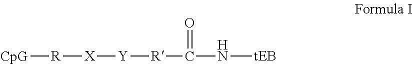

wherein CpG is a CpG oligodeoxynucleotide; tEB is a truncated Evans Blue dye; X is selected from amine, sulfur, carbonyl, or hydroxy amine; Y is selected from ester, amine, aliphatic, or a pyrrolidine dione; or X and Y combine to form a triazole or cyclooctatriazole; and each of R and R′ independently is selected from an aliphatic linker, an amide linker, an alkylene oxide linker, a peptide linker, or an oligonucleotide linker. In particular disclosed embodiments, tEB group has a structure satisfying Formula II

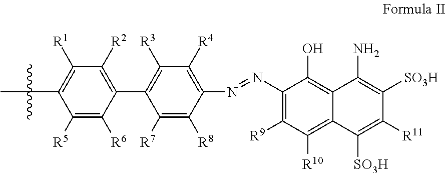

wherein each of R¹, R², R³, R⁴, R⁵, R⁶, R⁷, R⁸, R⁹, R¹⁰, and R¹¹ independently is selected from hydrogen, halogen, hydroxyl, cyano, aliphatic, heteroaliphatic, haloaliphatic, or haloheteroaliphatic.

In particular disclosed embodiments, the chemical conjugate can have a structure selected from those shown below.

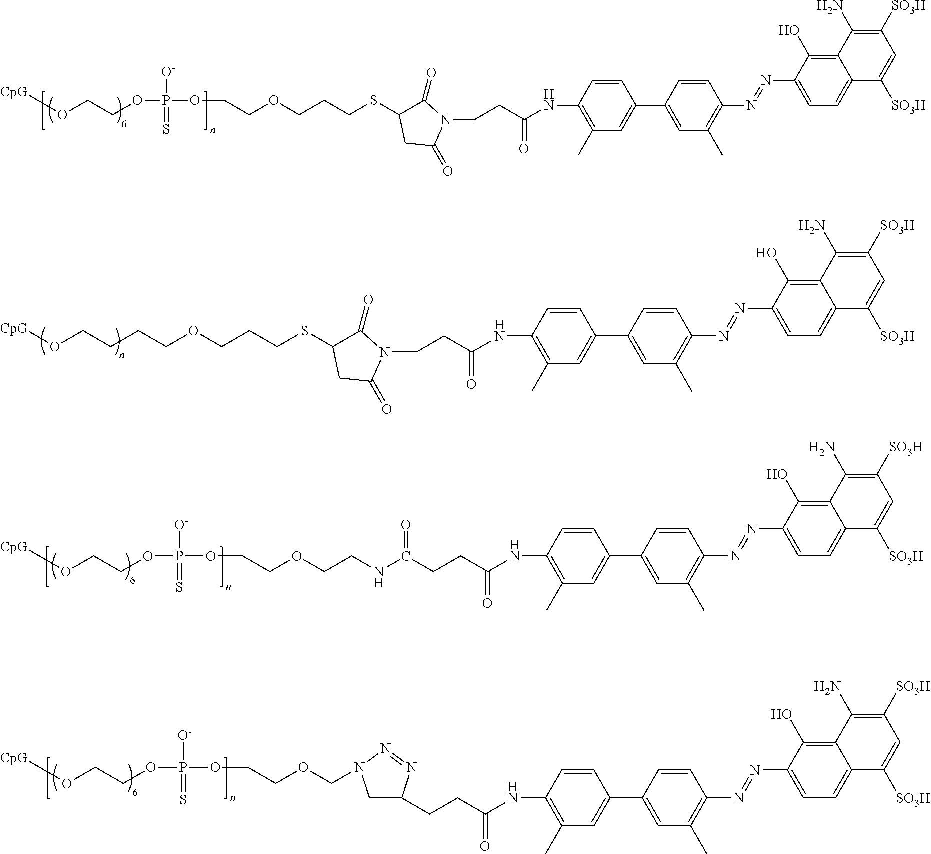

In some embodiments, disclosed are immunomodulatory compositions including the chemical conjugates described herein. The chemical conjugates and immunomodulatory compositions thereof can be used in methods to induce an immune response in a subject.

In some embodiments, the chemical conjugates and immunomodulatory compositions can be used to treat or prevent cancer. In other embodiments, the chemical conjugates and immunomodulatory compositions can be used to treat or prevent an infection.

The foregoing and other features and advantages of the invention will become more apparent from the following detailed description of several embodiments which proceeds with reference to the accompanying figures.

### BRIEF DESCRIPTION OF THE FIGURES

FIGS. 1A-1B. Schematic illustration of AlbiVax for LN-targeted delivery of molecular vaccines and combination cancer immunotherapy. (a) AlbiVax efficiently delivered molecular vaccines to LNs, via leveraging endogenous albumin as nanocarriers. AlbiVax was engineered by site-specifically conjugating the repurposed albumin-binding EB derivatives with CpG and subunit antigens, including TAA or tumor-specific neoantigen discovered via exosome DNA sequencing for personalized immunotherapy. AlbiCpG was synthesized by conjugating MEB with thiol-modified CpG, with HEG as tunable linkers. AlbiAg was synthesized via conjugation of MEB with Ag that was modified with an N-terminal cysteine. (b) The avid binding of subcutaneously (s.c.)-injected AlbiVax to endogenous albumin dramatically enhanced the efficiency of LN-targeted vaccine delivery via lymph draining and prolonged retention in LNs. AlbiCpG and AlbiAg bound to albumin were co-delivered into APCs, leading to potent stimulation of APCs, antigen cross presentation, and clonal expansion of antigen-specific CD8+ CTLs, thereby eliciting durable and potent anti-tumor immunity. AlbiVax upregulated the expression of PD-1 on these CTLs, and combination of AlbiVax and anti-PD-1 were studied for synergistic cancer immunotherapy in established primary tumors and lung metastatic tumors.

FIGS. 2A-2B. Characterization of MEB. (A) NMR spectrum of MEB. ¹H NMR (300 MHz, MeOD) δ 8.71 (s, 1H), 7.99 (d, J=9.9 Hz, 1H), 7.93 (d, J=8.5 Hz, 1H), 7.59 (d, J=8.7 Hz, 1H), 7.54 (s, 1H), 7.49 (s, 1H), 7.48-7.37 (m, 2H), 7.35 (t, J=6.7 Hz, 1H), 7.15 (d, J=9.9 Hz, 1H), 6.87 (s, 1H), 3.92 (t, J=6.8 Hz, 2H), 2.75 (t, J=6.8 Hz, 2H), 2.52 (s, 3H), 2.26 (s, 3H). (B) LC-MS analysis of MEB.

FIGS. 3A-3B. LC-MS results verifying the conjugation of MEB-CpG.

FIGS. 4A-4E. Recovery of MEB fluorescence and prolonged MEB fluorescence lifetime upon albumin binding of MEB-CpG. (A) Flow cytometry results showing the MEB fluorescence of HSA-coated beads in the presence of MEB-CpG, indicating binding of MEB-CpG to HSA. (B-D) Fluorescence of free MEB was enhanced upon conjugation on DNA (CpG and GpC), and the fluorescence of MEB in MEB-CpG (b) or MEB-GpC (C) was further enhanced in the presence of HSA, suggesting the binding of MEB-CpG with HSA. The fluorescence of MEB and MEB-CpG showing the blue shift of the fluorescence spectrum of MEB-CpG relative to free MEB. (Ex: 530 nm. MEB or equivalent: 1 μM. HSA: 10 μM) (d) Prolonged FL lifetime in the presence of HSA again suggests binding of AlbiCpG with HSA. (Ex: 480 nm. MEB or equivalent: 1 μM. HSA: 10 M) (E) Results of ELISA analysis of the culture medium of in vitro RAW264.7 macrophages demonstrate that MEB-CpG with MEB modified on the 3′-end of CpG maintained its immunostimulatory activity either alone or premixed with human serum albumin (HSA); MEB-CpG conjugate with MEB modified on the 5′-end of CpG dramatically abrogated the immunostimulatory potency of CpG; and that AlibiGpC control were inactive for immunostimulation, ruling out the immunogenicity of MEB moiety. Data show mean±s.e.m. of 3 independent experiments. ****p<0.0001, ***p<0.001, ns: not significant (p>0.05) by one-way ANOVA with Bonferroni post-test. Significance was compared between the group of (CpG 20 nM) and the corresponding other groups.

FIG. 5A-5C. Screening AlbiVax for LN-targeted CpG delivery with reduced systemic toxicity. (A) Structures, formulations, and radiolabeling strategies of CpG derivatives for PET-based screening. (B) Representative coronal PET images showing FVB mice at 6 h post s.c. injection of CpG derivatives at tail base. Four MEB-CpG derivatives efficiently delivered CpG to LNs, relative to free CpG, PEG-CpG, and IFA(CpG). White arrow heads mark IN and AX LNs. (C) Amounts of CpG derivatives in IN and AX LNs quantified from 3D-reconstructed, decay-corrected PET images (n=4-8). The IFA(CpG) signal in LNs was too low to be accurately quantified. Data show mean±standard error of mean (s.e.m.) of 2 independent experiments. ***p<0.001, **p<0.01, *p<0.05, ns: not significant (p>0.05) by one-way ANOVA with Bonferroni post-test. Color-matched asterisks in (c) indicate statistically significant differences between the corresponding compound with MH₂C.

FIG. 6. Coronal (coro), transverse (trans), and 3D projection (proj) of PET images showing efficient LN-targeted delivery and whole-body distribution of MH₂C over 3 days post injection. White arrow heads mark IN and AX LNs. The scale bar for 3D proj was based on the maximal signal intensity in each image. (Injection dose: 120 μCi; ID: injection dose).

FIGS. 7A-7B. (A) Coronal, transverse, and sagittal views of PET scanning of mice administered with Cu⁶⁴-labeled AlbiCpG (MEB-(HEG)₂-CpG), showing the retention of MEB-(HEG)₂-CpG in the injection depot and the gradual draining away from this depot. (B) Quantification of MEB-(HEG)₂-CpG retained in the injection depot over 3 days post administration.

FIGS. 8A-8E. Biodistribution and LN retention of CpG on day3 and day5, as determined by ROI signal quantification of PET images and γ-counting of isolated organs. (A) PET quantification of the % ID of different forms of CpG in major organs on Day3. (B) Detailed PET quantification of the % ID of different forms of CpG in IN and AX LNs on Day3. (C) γ-counting quantification of the % ID/g of different forms of CpG in LNs on Day3. (D) γ-counting quantification of the % ID/g of different forms of CpG in major organs on Day5. (E) γ-counting quantification of the % ID/g of different forms of CpG in LNs on Day5. Due to signal decay over 5 days, the radioactivity was too low to be detected by PET on day5, so the % injection dose on Day5 was not determined by PET. ID: injection dose; ID/g: injection dose/gram of tissue.

FIGS. 9A-9B. (a) Intrinsic fluorogenecity of MEB demonstrated targeted delivery of AlbiCpG into LNs. (b) AlbiCpG was retained in draining inguinal LNs for up to a week post injection. Shown above are the photographs of IN LNs collected on the corresponding days post injection, and on the bottom are the fluorescence images of LN tissue slices showing the distribution of AlbiCpG in LNs collected on the corresponding dates.

FIGS. 10A-10B. Intranodal and intracellular delivery of AlbiVax. (A) Photographs of non-cleared and cleared draining LNs using PACT. (B) Light sheet fluorescence microscopy images showing the 3D intranodal distribution of Alexa488-labeled AlbiCpG in a whole IN LN, which was resected from a C57BL/6 mouse 1 day after vaccination with 5 nmole AlbiCpG (left: 3D projection; right: cross-sections). Substantial AlbiCpG was located within or near the subcapsular sinus and around B cell follicles.

FIGS. 11A-11F. Intracellular delivery of albumin/AlbiCpG into APCs in vivo and in vitro. (A) Representative flow cytometry results showing the uptake of AlbiCpG-Alexa555 and/or MSA-FITC in B220⁺ B cells, CD11c⁺ DCs, and F4/80⁺ macrophages, 1 day (D1) and 3 days (D3) post vaccination with premixed MSA-FITC/AlbiCpG-Alexa555. (B) Median fluorescence intensity (MFI) of MSA-FITC in the above B220⁺ B cells, CD11c⁺ DCs, and F4/80⁺ macrophages. The MFI of AlbiCpG will be discussed below. (C-E) Efficient in vitro uptake of AlbiCpG into BMDCs and RAW264.7 macrophages was demonstrated by confocal microscopy (c), quantitative γ-counting using ⁶⁴Cu-labeled AlbiCpG (D), and flow cytometry using MEB fluorescence (E). (F) Same study as shown in FIG. 3E showing super-resolution confocal microscopy of one BMDC, with individual and merged fluorescence/bright-field channels.

FIGS. 12A-12C. (A) Representative flow cytometry plots showing the up-regulated expression of CD80 in DCs and macrophages of draining IN LNs. C57BL/6 mice were s.c. injected with AlbiCpG-Alexa555, and LN-residing APCs were analyzed on day3 post injection. (B) AlbiCpG induced local lymphadenopathy, in which LNs swelled resulting from the buildup of lymph and cells in LNs. C57BL/6 mice were s.c. injected at the base of tail (dose: 5 nmole/mouse), on day0 and day3, and organs were collected on day6. (C) In vitro immunostimulation of AlbiCpG in APCs was also demonstrated by up-regulated expression of co-stimulatory factors CD86 and/or CD80 in RAW264.7 and BMDCs (concentrations: 100 nM; treatment time: 14 h).

FIG. 13. ELISA analysis of the culture medium of in vitro cells demonstrate that AlbiCpG, either alone or premixed with albumin, stimulated BMDCs and RAW264.7 cells to produce proinflammatory factors at the comparable levels as free CpG.

FIGS. 14A-14C. Efficient co-delivery of AlbiVax (AlbiCpG+AlbiCSIINFEKL (SEQ ID NO: 85) to LN-residing APCs. On D1 and D3 post s.c. injection of AlbiCpG-Alexa555+AlbiCSIINFEK_{(FITC)}L (SEQ ID NO: 85, AlbiSIIN) at the tail base of C57BL/6 mice, flow cytometry (representative plots in A) was used to analyze the MFI (B) and fraction (C) of draining IN LN-residing B220⁺ B cells, CD11c⁺ DCs, and F4/80⁺ macrophages that took up AlbiCpG and/or AlbiSIIN. (i) Super-resolution confocal microscopy images showing intracellular co-delivery of AlbiCpG-Alexa555 (200 nM)+AlbiSIINFEK_{(FITC)}L (SEQ ID NO: 84) (200 nM) in one BMDC after 2-h incubation.

FIGS. 15A-15C. AlbiCpG ameliorated the systemic toxicity of CpG (n=4) as shown by ameliorated splenomegaly (A) on day6 (dose: 5 nmole CpG on days 0 and 3) and lower IL-6 and IL-12p40 levels in blood (B, C) (5 nmole CpG) induced by AlbiCpG relative to free CpG.

FIG. 16A-16C. AlbiVax (AlbiCpG+OVA) induced potent and durable T cell response for tumor immunotherapy. (A-C) C57BL/6 mice (n=5-8) were s.c. vaccinated at the tail base on days 0, 14, and 28 with AlbiCpG (2 nmole CpG equivalents)+OVA (10 μg), followed by immune analysis on day21, 35, and 70, and 1° tumor challenge on day 71. (A) Representative flow cytometry scatter plots (left) and frequency (right) of SIINFEKL-specific CD8⁺ T cells in peripheral blood on day21 stained using phycoerythrin (PE)-labelled H-2K^{b}-SIINFEKL tetramer. (C) Higher frequency and level (MFI) of PD-1 expression on SIINFEKL (SEQ ID NO: 84)-specific CD8 T cells than that on total CD8⁺ T cells of peripheral blood on day21. (Two-tailed paired t-test) (B) Frequencies of SIINFEKL⁺ CD8⁺ T cells in peripheral blood over 70 days post priming.

FIG. 17. AlbiVax (AlbiCpG+OVA) induced potent production of antigen-specific antibodies. C57BL/6 mice (n=4-5) were s.c. vaccinated at the tail base on day0 and day14 with AlbiVax (2 nmole CpG equivalents, 10 μg OVA), followed by analysis of OVA-specific antibody titers on day21. Shown are serum titers of IgG2a, IgM, IgG and IgG1 in vaccinated mice. AlbiCpG was superior in the production of IgG2a, which is effective for tumor therapy. IFA-emulsified CpG+OVA [IFA(CpG+OVA)] was superior to AlbiCpG in the overall IgG and IgG1 response.

FIG. 18A-18B. Tumor growth curve (A) and mouse survival (B) after EG7.OVA tumor challenge on vaccinated mice. Vaccinated mice were subject to 10 s.c. tumor challenge on the right shoulder on day71 with 3×10⁵ EG7.OVA cells, and survived AlbiVax-vaccinated mice were subject to 20 s.c. tumor challenge on the right flank on day211 with 1×10⁶ EG7.OVA cells.

FIG. 19. AlbiCpG+OVA partially eradicated established EG7.OVA tumor. C57BL/6 mice (n=6-8) were s.c. inoculated with EG7.OVA cells (3×10⁵) on day0, and treated with AlbiCpG+OVA (2 nmole CpG equivalents, 20 μg OVA) on day6, 12, and 18. Treatment with anti-CD8, but not anti-CD4 or anti-NK1.1 (dose: 200 μg, on days 6, 9, 12, 15, and 18) abrogated the therapeutic efficacy of AlbiVax. ***p<0.001, **p<0.01, *p<0.05, ns: not significant (p>0.05), by one-way ANOVA with Bonferroni post-test, unless denoted otherwise. Data show mean±s.e.m. of 2-3 independent experiments. Asterisks in indicate statistically significant differences between AlbiCpG and other groups.

FIG. 20A-20C. Experiment outline (left, A): C57BL/6 mice (n=5-7) were inoculated with 3×10⁵ EL4 cells on the left shoulder and 3×10⁵ EG7.OVA cells on the right shoulder on day0, treated with AlbiVax or controls (2 nmole CpG, 20 μg OVA) on day3 and day9. Right: growth curves of EL4 tumors and EG7.OVA tumors. (B) Tumor sizes of EL4 and EG7.OVA on day 19 post tumor inoculation. ***P<0.001, **P<0.01, *P<0.05, ns: not significant (P>0.05), by one-way ANOVA with Bonferroni post-test, unless denoted otherwise. Data show mean±s.e.m. Asterisks indicate statistically significant differences between AlbiCpG and other groups. (C) Mouse weight monitored over the course of treatment. No significant mouse weight change or morbidity was observed during the treatment

FIG. 21A-21C. TEM (A) and AFM (B) images of albumin/AlbiTrp2 nanocomplexes. The length-height graph in (B) was shown for the marked crossline in AFM image. (C) Albumin binding of AlbiTrp2 recovered the MEB fluorescence.

FIG. 22A-22B. Representative coronal (coro), transverse (trans), and 3D projection (proj) of PET images (A) post injection of AlbiTrp2, free Trp2, and IFA(Trp2) and quantification (B) of AlbiTrp2 and Trp2 in draining IN and AX LNs of FVB mice. White arrows mark LNs.

FIG. 23A-23D. Representative PET images (A) and the corresponding quantification (B) showing the uptake of AlbiCpG (MEB-(HEG)₂-CpG) in draining IN LNs and AX LNs of B16F10 tumor-bearing C57BL/6 mice. Tumor-draining LNs, the AX LNs on the right of mouse body, showed low vaccine signal due to blocked lymphatic drainage in the big tumor tissues. (C-D) Representative PET images (c) and the corresponding quantification (D) showing the uptake of AlbiTrp2 in LNs (IN and AX) of B16F10 tumor-bearing C57BL/6 mice. (Coro: coronal; trans: transverse; proj: projection; ID: injection dose; ID/g: ID/gram of organ weight)

FIG. 24A-24C. AlbiVax (AlbiCpG+AlbiTrp2) for melanoma immunotherapy. B16F10 tumor growth after treatment with AlbiCpG+AlbiTrp2 alone (A), or with double combination of AlbiCpG+AlbiTrp2 and anti-PD-1 (B) or with triple combination of AlbiCpG+AlbiTrp2, anti-PD-1 and Abraxane (C). C57BL/6 mice were s.c. inoculated with 3×10⁵ B16F10 cells, treated with AlbiCpG (2 nmole CpG equivalents)+AlbiTrp2 (20 μg) (days 6, 12, and 18), anti-PD-1 antibody every 3 days from day6 for 5 times (dose: 200 μg), and Abraxane on days 6, 12, and 18 (dose: 20 mg/kg). Data show mean±s.e.m. of 2-3 independent experiments. ***p<0.001, **p<0.01, *p<0.05, ns: not significant (p>0.05) by one-way ANOVA with Bonferroni post-test.

FIG. 25A-25D. Representative coronal and transverse of PET images (A) at 6 h post injection and quantification (B) of AlbiAdpgk, free Adpgk, and IFA(Adpgk) in IN and AX LNs of FVB mice (n=4/group). White arrows mark the LNs. (C) Quantified biodistribution of ⁶⁴Cu-labeled AlbiAdpgk in organs by γ-counting of excised organs at 48 h post injection. (D) % ID at injection sites quantified from decay-corrected PET scans.

FIG. 26A-26C. C57BL/6 mice (n=5/group) were vaccinated with AlbiVax (AlbiCpG+AlbiAdpgk) (2 nmole CpG and 20 μg AlbiAdpgk) on day0 and day14, followed by flow cytometric analysis of H-2D^{b}-ASMTNMELM (SEQ ID NO: 86) tetramer⁺ CD8⁺ T cells (A, B) and PD-1 expression on CD8⁺ T cells (C) in peripheral blood on day21.

FIG. 27A-27C. MC38 tumor growth after treatment with AlbiCpG+AlbiAdpgk alone (A) or in combination with anti-PD-1 (B). (C) Individual tumor growth curves. C57BL/6 mice were s.c. inoculated with 3×10⁵ MC38 cells, treated with AlbiCpG (2 nmole CpG equivalents)+AlbiAdpgk (20 μg) on days 6, 12, and 18) and anti-PD-1 antibody every 3 days from day6 for 5 times (dose: 200 μg). Results were pooled from 3 independent experiments. Data show mean±s.e.m. of 2-3 independent experiments. ***p<0.001, **p<0.01, *p<0.05, ns: not significant (p>0.05) by one-way ANOVA with Bonferroni post-test.

FIG. 28 illustrates a representative chemical conjugate as described herein.

FIG. 29 illustrates another representative chemical conjugate as described herein.

FIG. 30 illustrates yet another representative chemical conjugate as described herein.

FIG. 31 illustrates yet another representative chemical conjugate as described herein.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [4239-96721-03_Sequence.txt, created on Oct. 30, 2018, 18.5 KB, which is incorporated by reference herein. In the accompanying sequence listing:

SEQ ID NOs: 1-36 are K-type CpG oligodoexynucleotides (ODNs).

SEQ ID NOs: 37-64 are D-type CpG ODNs.

SEQ ID NOs: 65-69 are C-type ODNs.

SEQ ID NOs: 70-86 are exemplary antigens.

### DETAILED DESCRIPTION OF SEVERAL EMBODIMENTS

Evans Blue, a symmetric azo dye, has high binding affinity to albumin, and can be used for clinic measurement of blood plasma volume (el-Sayed et al., 1995, Clinical and laboratory haematology 17(2): 189-194) and preclinical visualization of sentinel LNs (Tsopelas and Sutton, 2002, Journal of nuclear medicine: official publication, Society of Nuclear Medicine 43(10):1377-1382). Despite the termination of the above clinical use due to toxicity at high dose, the relatively low doses of MEB used in vaccination-based immunotherapy have low toxicity. The albumin-binding ability of MEB was utilized with CpG ODN and tumor-specific antigens, in order to leverage endogenous albumin nanocarriers that increased the safety and the potency of molecular vaccines. Molecular vaccines are disclosed herein that leverage endogenous albumin nanocarriers by conjugation of CpG ODN with functionalized truncated Evans Blue (MEB). Albumin-binding MEB-vaccine conjugates (“AlbiVax”) enable efficient delivery and prolonged retention in LNs, and reduced the systemic toxicity and enhanced the therapeutic potency of molecular vaccines.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Adjuvant: A vehicle used to enhance antigenicity. Adjuvants include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL, immune stimulating complex (ISCOM) matrix, and toll-like receptor (TLR) agonists, such as TLR-9 agonists, Poly I:C, or PolyICLC. The person of ordinary skill in the art is familiar with adjuvants (see, e.g., Singh (ed.) Vaccine Adjuvants and Delivery Systems. Wiley-Interscience, 2007).

Administration: Administration of a chemical conjugate or immunomodulatory composition can be by any route known to one of skill in the art. Administration can be local or systemic. Examples of local administration include, but are not limited to, topical administration, subcutaneous administration, intramuscular administration, intrathecal administration, intrapericardial administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes intra-thecal injection, intra-cranial injection or delivery to the cerebral spinal fluid.

Systemic administration includes any route of administration designed to distribute a chemical conjugate and/or immunological compound or immunomodulatory composition widely throughout the body via the circulatory system. Thus, systemic administration includes, but is not limited to intra-arterial and intravenous administration. Systemic administration also includes, but is not limited to, oral administration, subcutaneous administration, intramuscular administration, or parenteral administration, when such administration is directed at absorption and distribution throughout the body by the circulatory system.

Aliphatic: A hydrocarbon, or a radical thereof, having at least one carbon atom to 50 carbon atoms, such as one to 25 carbon atoms, or one to ten carbon atoms, and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atoms 50 carbon atoms, such as two to 25 carbon atoms, or two to ten carbon atoms, and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: An alkyl group as defined above with the indicated number of carbon atoms covalently bound to the group it substitutes by an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “Alkylthio” or a “thioalkyl” group is an alkyl group as defined above with the indicated number of carbon atoms covalently bound to the group it substitutes by a sulfur bridge (—S—). Similarly, “alkenyloxy”, “alkynyloxy”, and “cycloalkyloxy” refer to alkenyl, alkynyl, and cycloalkyl groups, in each instance covalently bound to the group it substitutes by an oxygen bridge (—O—).

Alkyl: A saturated monovalent hydrocarbon having at least one carbon atom to 50 carbon atoms, such as one to 25 carbon atoms, or one to ten carbon atoms, wherein the saturated monovalent hydrocarbon can be derived from removing one hydrogen atom from one carbon atom of a parent compound (e.g., alkane). An alkyl group can be branched, straight-chain, or cyclic (e.g., cycloalkyl). In some embodiments, an alkyl group can have from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g. C₁-C₈alkyl, C₁-C₄alkyl, and C₁-C₂alkyl. When C₀-Cₙ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

Alkynyl: An unsaturated monovalent hydrocarbon having at least two carbon atoms 50 carbon atoms, such as two to 25 carbon atoms, or two to ten carbon atoms and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amide: —C(O)NR^{b}— wherein R^{b} is selected from hydrogen, aliphatic, heteroaliphatic, aryl, heteroaryl, or any combination thereof.

Amine: —NR^{a}—, wherein R^{b} is selected from hydrogen, alkyl, alkenyl, alkynyl, aryl, heteroalkyl, heteroalkenyl, heteroalkynyl, heteroaryl, and any combination thereof.

Animal: Living multi-cellular vertebrate organisms, a category that includes, for example, mammals and birds. The term mammal includes both human and non-human mammals. Similarly, the term “subject” includes both human and veterinary subjects. Therefore, the general term “subject” is understood to include all animals, including, but not limited to, humans, or veterinary subjects, such as other primates, dogs, cats, horses, and cows.

Anti-Inflammatory Agent: Any of various medications that decrease the signs and symptoms (for example, pain, swelling, or shortness of breath) of inflammation. Corticosteroids are exemplary potent anti-inflammatory medications. Nonsteroidal anti-inflammatory agents are also effective exemplary anti-inflammatory agents and do not have the side effects that can be associated with steroid medications.

Aryl: An aromatic carbocyclic group comprising at least five carbon atoms to 15 carbon atoms, such as five to ten carbon atoms, having a single ring or multiple condensed rings, which condensed rings can or may not be aromatic provided that the point of attachment is through an atom of the aromatic carbocyclic group.

Cancer: A malignant tumor that has undergone characteristic anaplasia with loss of differentiation, increase rate of growth, invasion of surrounding tissue, and is capable of metastasis. For example, thyroid cancer is a malignant tumor that arises in or from thyroid tissue, and breast cancer is a malignant tumor that arises in or from breast tissue (such as a ductal carcinoma). Residual cancer is cancer that remains in a subject after any form of treatment given to the subject to reduce or eradicate the cancer. Metastatic cancer is a tumor at one or more sites in the body other than the site of origin of the original (primary) cancer from which the metastatic cancer is derived. Cancer includes, but is not limited to, solid tumors.

Carboxyl: —C(O)O—.

Chemokine: A type of cytokine (a soluble molecule that a cell produces to control reactions between other cells) that specifically alters the behavior of leukocytes (white blood cells). Examples include, but are not limited to, interleukin 8 (IL-8), platelet factor 4, melanoma growth stimulatory protein, etc.

Chiral: Molecules, which have the property of non-superimposability of the mirror image partner.

Chemotherapy; chemotherapeutic agents: As used herein, any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating neoplasms such as solid tumors. In one embodiment, a chemotherapeutic agent is radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L., Berkery R. (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Chemotherapeutic agents include those known by those skilled in the art, including but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cisplatinum and the taxanes, such as taxol. Rapamycin has also been used as a chemotherapeutic.

CpG or CpG motif: A nucleic acid having a cytosine followed by a guanine linked by a phosphate bond in which the pyrimidine ring of the cytosine is unmethylated. The term “methylated CpG” refers to the methylation of the cytosine on the pyrimidine ring, usually occurring at the 5-position of the pyrimidine ring. A CpG ODN is an ODN that is at least about ten nucleotides in length and includes an unmethylated CpG. CpG ODNs include both D and K-type ODNs (see below). CpG ODNs are single-stranded. The entire CpG ODN can be unmethylated or portions may be unmethylated. In one embodiment, at least the C of the 5′ CG 3′ is unmethylated.

Cytokine: The term “cytokine” is used as a generic name for a diverse group of soluble proteins and peptides that act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor α (TNFα), interleukin-6 (IL-6), interleukin-10 (IL-10), interleukin-12

D-type Oligodeoxynucleotide (D ODN): A D-type ODN is at least about 16 nucleotides in length, such as 16 to 30 nucleotides in length, and includes a sequence represented by the following formula:

(SEQ ID NO: 37)

5′ X₁X₂X₃ Pu₁ Py₂ CpG Pu₃ Py₄ X₄X₅X₆(W)_{M} (G)_{N}-3′

wherein the central CpG motif is unmethylated, Pu is a purine nucleotide, Py is a pyrimidine nucleotide, X and Ware any nucleotide, M is any integer from 0 to 10, and N is any integer from 4 to 8, wherein X₁X₂X₃ and X₄X₅X₆ are self-complementary. Additional detailed description of D ODN sequences and their activities can be found in Verthelyi et al., J. Immunol. 166:2372-2377, 2001, which is herein incorporated by reference. Generally D ODNs can stimulate a cellular immune response.

Halo or Halogen: Fluoro, chloro, bromo, or iodo.

Haloaliphatic: An aliphatic group wherein one or more hydrogen atoms, such as one to 10 hydrogen atoms, independently is replaced with a halogen atom, such as fluoro, bromo, chloro, or iodo.

Haloalkyl: Both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

Haloalkoxy: A haloalkyl group as defined above attached through an oxygen bridge (oxygen of an alcohol radical).

Heteroaryl: An aryl group comprising at least one heteroatom to six heteroatoms, such as one to four heteroatoms, which can be selected from, but not limited to oxygen, nitrogen, sulfur, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group.

Immune response: A response of a cell of the immune system, such as a B cell or T cell to a stimulus. In one embodiment, the response is an inflammatory response.

Immunostimulatory CpG motifs: Immunostimulatory sequences that trigger macrophages, monocytes and lymphocytes to produce a variety of pro-inflammatory cytokines and chemokines. CpG motifs are found in bacterial DNA. The innate immune response elicited by CpG DNA reduces host susceptibility to infectious pathogens, and can also trigger detrimental inflammatory reactions. Immunostimulatory CpG motifs are found in “D” and “K” type ODNs (see, for example PCT Publication No. WO 01/51500, published on Jul. 19, 2001).

Inflammation: A localized protective response elicited by injury to tissue that serves to sequester the inflammatory agent. Inflammation is characterized by the appearance in or migration into any tissue space, unit or region of any class of leukocyte in numbers that exceed the number of such cells found within such region of tissue under normal (healthy) circumstances. Inflammation is orchestrated by a complex biological response of vascular tissues to harmful stimuli, such as pathogens, damaged cells, or irritants. It is a protective attempt by the organism to remove the injurious stimuli as well as initiate the healing process for the tissue. An inflammatory response is an accumulation of white blood cells, either systemically or locally at the site of inflammation. The inflammatory response may be measured by many methods well known in the art, such as the number of white blood cells, the number of polymorphonuclear neutophils (PMN), a measure of the degree of PMN activation, such as luminal enhanced-chemiluminescence, or a measure of the amount of cytokines present. Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

K-type CpG Oligodeoxynucleotide (K ODN): An ODN including an unmethylated CpG motif that has a sequence represented by the formula:

(SEQ ID NO: 1)

5′ N₁N₂N₃D-CpG-WN₄N₅N₆ 3′

wherein the central CpG motif is unmethylated, D is T, G or A, W is A or T, and N₁, N₂, N₃, N₄, N₅, and N₆ are any nucleotides. In one embodiment, D is a T. Additional detailed description of K ODN sequences and their activities can be found in the description below. Generally K ODNs can stimulate a humoral response. For example, K ODNs stimulate the production of immunoglobulins, such as IgM and IgG. K ODNs can also stimulate proliferation of peripheral blood mononuclear cells and increase expression of IL-6 and/or IL-12, amongst other activities. In several embodiments, K ODNs are about 10 to about 30 nucleotides in length.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). In some embodiments, for coding nucleic acids, when single stranded, the nucleic acid can be the sense strand or the antisense strand. Nucleic acids can include natural nucleotides (such as A, T/U, C, and G), and can include analogs of natural nucleotides, such as labeled nucleotides. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. A linear nucleic acid molecule has a 5′ end and a 3′ end.

Oligonucleotide, Oligodeoxynucleotide or “oligo”: Multiple nucleotides (i.e., molecules comprising a sugar (e.g., ribose or deoxyribose) linked to a phosphate group and to an exchangeable organic base, which is either a substituted pyrimidine (Py) (e.g., cytosine (C), thymine (T) or uracil (U)) or a substituted purine (Pu) (e.g., adenine (A) or guanine (G)). The term “oligonucleotide” as used herein refers to both oligoribonucleotides (ORNs) and oligodeoxyribonucleotides (ODNs). The term “oligonucleotide” also includes oligonucleosides (i.e., an oligonucleotide minus the phosphate) and any other organic base polymer. Oligonucleotides can be obtained from existing nucleic acid sources (e.g., genomic or cDNA), but are preferably synthetic (i.e., produced by oligonucleotide synthesis).

A “stabilized oligonucleotide” is an oligonucleotide that is relatively resistant to in vivo degradation (for example via an exo- or endo-nuclease). In one embodiment, a stabilized oligonucleotide has a modified phosphate backbone. One specific, non-limiting example of a stabilized oligonucleotide has a phophorothioate modified phosphate backbone (wherein at least one of the phosphate oxygens is replaced by sulfur). Other stabilized oligonucleotides include: nonionic DNA analogs, such as alkyl- and aryl-phosphonates (in which the charged phosphonate oxygen is replaced by an alkyl or aryl group), phophodiester and alkylphosphotriesters, in which the charged oxygen moiety is alkylated. Oligonucleotides which contain a diol, such as tetraethyleneglycol or hexaethyleneglycol, at either or both termini have also been shown to be substantially resistant to nuclease degradation.

An “immunostimulatory oligodeoxynucleotide,” “immunostimulatory CpG containing ODN,” “CpG ODN,” refers to an ODN, which contains a cytosine, guanine dinucleotide sequence. In one embodiment, CpG ODN stimulates (e.g. has a mitogenic effect or induces cytokine production) vertebrate immune cells. CpG ODN can also stimulate angiogenesis. The cytosine, guanine is unmethylated. This includes K and D ODN.

An “oligonucleotide delivery complex” is an oligonucleotide associated with (e.g., ionically or covalently bound to; or encapsulated within) a targeting means (e.g., a molecule that results in a higher affinity binding to a target cell (e.g, B-cell or natural killer (NK) cell) surface and/or increased cellular uptake by target cells). Examples of oligonucleotide delivery complexes include oligonucleotides associated with: a sterol (e.g., cholesterol), a lipid (e.g., cationic lipid, virosome or liposome), or a target cell specific binding agent (e.g., a ligand recognized by a target cell specific receptor). Preferred complexes must be sufficiently stable in vivo to prevent significant uncoupling prior to internalization by the target cell. However, the complex should be cleavable or otherwise accessible under appropriate conditions within the cell so that the oligonucleotide is functional. (Gursel, J. Immunol. 167: 3324, 2001).

Parenteral: Administered outside of the intestine, e.g., not via the alimentary tract. Generally, parenteral formulations are those that will be administered through any possible mode except ingestion. This term especially refers to injections, whether administered intravenously, intrathecally, intramuscularly, intraperitoneally, intraarticularly, or subcutaneously, and various surface applications including intranasal, intradermal, and topical application, for instance.

Pharmaceutically acceptable salts: Derivatives of the disclosed chemical conjugates and/or immunological compounds in which the parent compound is modified by making inorganic and organic, non-toxic, acid or base addition salts thereof. The salts of the present chemical conjugates and/or immunological compounds can be synthesized from a parent compound that contains a basic or acidic moiety by conventional chemical methods. Generally, such salts can be prepared by reacting free acid forms of these chemical conjugates and/or immunological compounds with a stoichiometric amount of the appropriate base (such as Na, Ca, Mg, or K hydroxide, carbonate, bicarbonate, or the like), or by reacting free base forms of these chemical conjugates and/or immunological compounds with a stoichiometric amount of the appropriate acid. Such reactions are typically carried out in water or in an organic solvent, or in a mixture of the two. Generally, non-aqueous media such as ether, ethyl acetate, ethanol, isopropanol, or acetonitrile are used, where practicable. Salts of the present chemical conjugates and/or immunological compounds further include solvates of the chemical conjugates and/or immunological compounds and of the chemical conjugates and/or immunological compounds salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth, Editors, Wiley-VCH, 2002.

Pharmaceutical agent or drug: A chemical compound or immunomodulatory composition capable of inducing a desired therapeutic or prophylactic effect when properly administered to a subject. Pharmaceutical agents include, but are not limited to, anti-infective agents, anti-inflammatory agents, bronchodilators, enzymes, expectorants, leukotriene antagonists, leukotriene formation inhibitors, and mast cell stabilizers.

Pharmaceutically acceptable carriers: The pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of the chemical conjugates and/or immunological compounds herein disclosed. Pharmaceutical carriers are any excipient, diluent, or vehicle, that is useful in preparing an immunomodulatory composition that is generally safe, non-toxic and neither biologically nor otherwise undesirable, and includes a carrier that is acceptable for human or veterinary pharmaceutical use.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, immunomodulatory compositions to be administered can contain minor amounts of non-toxic auxiliary substances, such as wetting or emulsifying agents, preservatives, and pH buffering agents and the like, for example sodium acetate or sorbitan monolaurate.

Preventing or treating: Inhibiting a disease refers to inhibiting the full development of a disease, for example in a person who is at risk for a disease such as one caused by an infectious agent, or for a tumor. An example of a person at risk for infection is someone with a family member with the infection. An example of a person at risk for cancer is someone with a family history of the cancer. Another example of a person at risk for a disease is someone who has recognized risk factors for the disease, such as human papilloma virus (HPV) for cervical cancer, tobacco smoking for lung cancer, or intravenous drug use for hepatitis B virus (HBV) or a human immunodeficiency virus (HIV) infection. Inhibiting a disease process includes preventing the development of the disease. “Treatment” refers to a therapeutic intervention that ameliorates a sign or symptom of a disease or pathological condition, such as after it has begun to develop.

Prime-boost vaccination: An immunotherapy including administration of a first immunomodulatory composition (the prime) followed by administration of an additional immunomodulatory composition (the boost) to a subject to induce an immune response. The boost is administered to the subject after the prime; the skilled artisan will understand a suitable time interval between administration of the prime and the boost, and examples of such timeframes are disclosed herein. Additional administrations can be included in the prime-boost protocol, for example a second boost.

Stereoisomers: Compounds having identical chemical constitution, but that differ with regard to the arrangement of the atoms or groups in space. Diastereomer: A stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis, crystallization in the presence of a resolving agent, or chromatography, using, for example a chiral HPLC column. Enantiomers: Two stereoisomers of a compound, which are non-superimposable mirror images of one another. A 50:50 mixture of enantiomers is referred to as a racemic mixture or a racemate, which may occur where there has been no stereoselection or stereospecificity in a chemical reaction or process. Racemic mixture or Racemate: An equimolar (or 50:50) mixture of two enantiomeric species, devoid of optical activity. A racemic mixture may occur where there has been no stereoselection or stereospecificity in a chemical reaction or process.

Substituted: Any one or more hydrogens on the designated atom or group is replaced with a selection from the indicated group, provided that the designated atom's normal valence is not exceeded. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. Any functional group disclosed herein and/or defined above can be substituted or unsubstituted, unless otherwise indicated herein.

Therapeutic agent: Used in a generic sense, it includes treating agents, prophylactic agents, and replacement agents. “Treatment” or “treating” means providing a chemical conjugate to a patient in an amount sufficient to measurably reduce any disease symptom, slow disease progression or cause disease regression. In certain embodiments treatment of the disease may be commenced before the patient presents symptoms of the disease. A protective immune response prevents signs and symptoms of a disease, such as one caused by a pathogenic agent.

Therapeutically effective amount: A “therapeutically effective amount” of an immunomodulatory composition means an amount effective, when administered to a patient, to provide a therapeutic benefit such as an amelioration of symptoms, decrease disease progression, or cause disease regression. A quantity of a specified chemical conjugate sufficient to achieve a desired effect in a subject being treated. A therapeutically effective amount of a chemical conjugate can be administered systemically or locally. In addition, an effective amount of a chemical conjugate can be administered in a single dose, or in several doses, for example daily, during a course of treatment. However, the effective amount of the chemical conjugate will be dependent on the preparation applied, the subject being treated, the severity and type of the affliction, and the manner of administration of the compound. The chemical conjugates and/or immunological compounds disclosed herein have equal applications in medical and veterinary settings. Therefore, the general term “subject” or “patient” is understood to include all animals, including, but not limited to, humans or veterinary subjects, such as other primates, dogs, cats, horses, and cows.

Tumor: An abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

Vaccine: A preparation of immunogenic material capable of stimulating an immune response, administered for the prevention, amelioration, or treatment of infectious or other types of disease. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response. In one specific, non-limiting example, a vaccine prevents and/or reduces the severity of the symptoms associated with influenza or HIV-1 infection compared to a control. A vaccine can be a subunit vaccine, a heat killed vaccine or an attenuated vaccine.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. A significant change is any detectable change that is statistically significant in a standard parametric test of statistical significance such as Student's T-test, where p<0.05.

### Chemical Conjugates and Immunomodulatory Compositions

Chemical conjugates are described using standard nomenclature. Unless defined otherwise, all technical and scientific terms used herein have the same meaning as is commonly understood by one of skill in the art to which this invention belongs. All chemical conjugates are understood to include all possible isotopes of atoms occurring in the chemical conjugates. Isotopes include those atoms having the same atomic number but different mass numbers. By way of general example, and without limitation, isotopes of hydrogen include tritium and deuterium and isotopes of carbon include ¹¹C, ¹³C, and ¹⁴C. Formulae I and II include all pharmaceutically acceptable salts of Formulae I and II.

A dash (“-”) that is not between two letters or symbols is used to indicate a point of attachment for a substituent and in some embodiments, a wavy line (“ [symbol] ”) can indicate where the component, functional group, or molecule is disconnected from another portion of the overall compound.

Chemical conjugates disclosed herein may contain one or more asymmetric elements such as stereogenic centers, stereogenic axes and the like, e.g., asymmetric carbon atoms, so that the chemical conjugates can exist in different stereoisomeric forms. These compounds can be, for example, racemates or optically active forms. For chemical conjugates with two or more asymmetric elements, these compounds can additionally be mixtures of diastereomers. For chemical conjugates having asymmetric centers, all optical isomers in pure form and mixtures thereof are encompassed. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent may be employed either alone or in combination.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

The chemical conjugates disclosed herein include an immunological compound, such as a CpG oligodeoxynucleotide (“CpG” or “CpG ODN”). The CpG ODN is preferably in the 5′ to 3′ orientation in all of the chemical conjugates disclosed herein.

In some embodiments, chemical conjugates of Evans Blue dye have structures satisfying Formula I illustrated below, or can be a pharmaceutically acceptable ester, amide, solvate, or salt thereof, or a salt of such an ester or amide or a solvate of such an ester amide or salt:

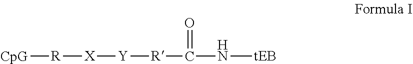

With reference to Formula I, “CpG” represents a CpG group as described herein. In particular disclosed embodiments, the R moiety of Formula I can be linked to the CpG moiety through a phosphorothioate moiety (e.g., —OP(S)(O⁻)O—), a PEG-functionalized phosphorothioate moiety (e.g., —Oₐ(CH₂)₂OP(S)(O⁻)O_{b}—, wherein the CpG is bound to Oₐ and R is bound to O_{b}) or a polymeric PEG-functionalized phosphorothioate moiety (e.g., —[Oₐ(CH₂)₂)ₙOP(S)(O⁻)O_{b}]_{n′}—, wherein the CpG is bound to Oₐ and R is bound to O_{b} and each of n and n′ independently can be 1 to 10, such as 1 to 8, or 1 to 6). Also with reference to Formula I, “tEB” represents a truncated Evans Blue dye or derivative thereof, which can have a Formula II, illustrated below:

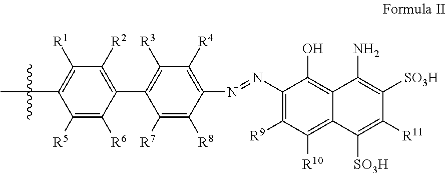

wherein the substituents R¹, R², R³, R⁴, R⁵, R⁶, R⁷, R⁸, R⁹, R¹⁰, and R¹¹ are selected independently from hydrogen, halogen, hydroxyl, cyano, aliphatic (e.g., C₁-C₆alkyl), heteroaliphatic (e.g., C₁-C₆alkoxy), haloaliphatic (e.g., C₁-C₆haloalkyl), and haloheteroaliphatic (e.g., C₁-C₆haloalkoxy). In one embodiment of Formula II, R¹ and R⁴ are each selected independently from halogen, hydroxyl, cyano, C₁-C₆alkyl, C₁-C₆alkoxy, C₁-C₆haloalkyl, and C₁-C₆haloalkoxy. In yet another embodiment of Formula II, R², R³, R⁵, R⁶, R⁷, R⁸, R⁹, R¹⁰, and R¹¹ are each hydrogen. In yet another embodiment of Formula II, R¹ and R⁴ are each C₁-C₆alkyl. In yet another embodiment of Formula II, R¹ and R⁴ are each methyl.

With further reference to Formula I, each of R and R′ independently can be selected from an aliphatic linker, an alkylene oxide linker, a peptide linker, an oligonucleotide linker, or a combination thereof. In some embodiments each of R and R′ can be optionally present. In particular disclosed embodiments, R can be coupled to the CpG group through the phosphorothioate groups described above and in some embodiments can be coupled to X directly or through one or more methylene groups. In some embodiments, R′ can be coupled to the carbonyl group illustrated in Formula I directly or through one or more methylene groups and further can be coupled to Y directly or through one or more methylene groups. X can be selected from amine (e.g., —NR^{a}—, wherein R^{a} is selected from hydrogen, aliphatic, or aryl), sulfur, carbonyl (e.g., —C(O)—), or hydroxyl amine (e.g.,

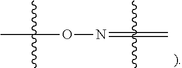 [SMILES: *ON=*]

Y can be selected from ester (e.g., —C(O)O—), carbonyl, amine (—NR^{a}—, wherein R^{a} is selected from hydrogen, aliphatic, or aryl), aliphatic (e.g.,

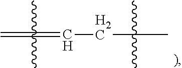 [SMILES: *CC=*]

or a pyrrolidine dione compound (e.g.,

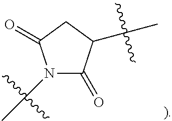 [SMILES: *C1CC(=O)N(*)C1=O]

In some embodiments, X and Y can combine to form a triazole moiety or a cyclooctatnriazoe moiety, such as through a cycloaddition reaction between X and Y. In such embodiments, X can be a triazine moiety (e.g., —R^{a}N—N═N—, wherein R^{a} is as recited above) and Y can be (1) a cyclooctane group fused to the two terminal nitrogen atoms of X, or (2) a —CH₂—CH₂— group fused to the two terminal nitrogen atoms of X. Thus, in such embodiments, the conjugate can have a Formula III, in which X and Y have been reacted via cycloaddition to form a “Z” group, which can be a triazole or a cyclooctatriazole group.

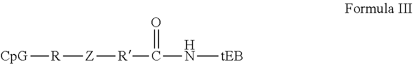

In some embodiments, one or both of R and R′ can be an oligonucleotide linker comprising one or more adenosine moieties, thymidine moieties, cytidine moieties, guanosine moieties, or a combination thereof. In particular disclosed embodiments, the oligonucleotide linkers can comprise a nucleotide chain having a formula (A)ₙ, (T)ₙ, (C)ₙ, (G)ₙ, (AT)ₙ, (AC)ₙ, (AG)ₙ, (TC)ₙ, (ATC)ₙ, (ATCG)ₙ, or (ATCGA)ₙ. In such embodiments, n can be selected from an integer that provides a linker having a length ranging from 0 to 200 nm, such as greater than 0 nm to 150 nm, or 1 nm to 100 nm, or 20 nm to 50 nm. In some embodiments, n ranges from 1 to about 200, such as 1 to about 150, or 1 to about 100, or 1 to about 50, or 1 to about 25, or 1 to about 10.

In some embodiments, one or both of R and R′ can be a peptide linker comprising one or more glycine peptides, alanine peptides, serine peptides, threonine peptides, or a combination thereof. In particular disclosed embodiments, the peptide linkers can comprise one or more peptides having a formula (G)ₙ, (A)ₙ, (S)ₙ, (T)ₙ, (GA)ₙ, (TA)ₙ, (SA)ₙ, (GAT)ₙ, (TAS)ₙ, (SATA)ₙ. In such embodiments, n can be selected from an integer that provides a linker having a length ranging from 0 to 200 nm, such as greater than 0 nm to 150 nm, or 1 nm to 100 nm, or 20 nm to 50 nm. In some embodiments, n ranges from 1 to about 200, such as 1 to about 150, or 1 to about 100, or 1 to about 50, or 1 to about 25, or 1 to about 10.

In some embodiments, one or both of R and R′ can be an aliphatic linker selected from alkyl, alkenyl, alkynyl or a combination thereof. In particular disclosed embodiments, the aliphatic linker can be an alkyl group having a formula —(C(R^{b})₂)ₙ—, wherein each R^{b} independently can be hydrogen or aliphatic. In particular disclosed embodiments, the aliphatic linker can be a C₁-C₂₀alkyl group, such as methyl, ethyl, propyl, butyl, pentyl, hexyl, septyl, octyl, and the like.

In some embodiments, one or both of R and R′ can be an alkylene oxide linker, such as a PEG linker. Any number of repeating alkylene oxide (e.g., ethylene glycol) units can be used to provide an R and/or R′ linker length ranging from 0 nm to 200 nm, such as greater than 0 nm to 150 nm, or 1 nm to 100 nm, or 20 nm to 50 nm. In such embodiments, a number of repeating alkylene oxide units can be used to achieve such lengths, such as 0 to 600 repeating alkylene oxide units, or from 1 to 600 repeating alkylene oxide units, such as 1 to 300 repeating alkylene oxide units, or 1 to 100 repeating alkylene oxide units, or 1 to 50 repeating alkylene oxide units, or 1 to 25 alkylene oxide units, or 1 to 10 repeating alkylene oxide units. In some embodiments, the alkylene oxide linker can be selected to have a molecular weight of 0 to about 35 KDa, such as greater than 0 to about 25 KDa, or about 60 Da to about 10 KDa, or about 60 Da to about 5 KDa.

In particular disclosed embodiments, about three alkylene oxide units (e.g., ethylene glycol units) has a length of about 1 nm and about 1 alkylene oxide units (e.g., ethylene glycol units) have a molecular weight of 60 Da.

In particular disclosed embodiments, the chemical conjugate can have a structure meeting any one of Formulas IV-IX illustrated below. In additional embodiments, the chemical conjugate can comprise a mixed linker system wherein R and R′ are different. Solely by way of example, if R is an alkyl linker, R′ can be selected from an alkylene oxide linker, a peptide linker, or oligonucleotide linker. Representative chemical conjugates with mixed linker systems can have structures satisfying any one of Formulas illustrated in Table 1, but the present disclosure is not limited to these particular embodiments and further contemplates other possible mixed linker combinations. With reference to Formulas IV-IX and those provided by Table 1, each n independently is an integer selected to provide an R linker length of greater than 0 nm to 200 nm and/or an R′ linker length of greater than 0 nm to 200 nm; and each m independently is 0 or 1. In some embodiments, n ranges from 1 to 600, such as from 1 to 300, or 1 to 150, or 1 to 100, or 1 to 50. Each of B and B′ independently is an oligonucleotide linker and each A and A′ independently is a peptide linker.

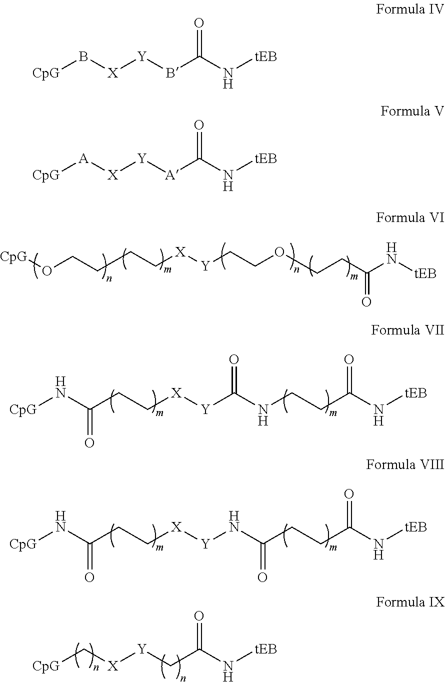

TABLE 1

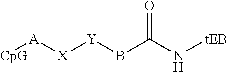

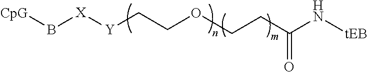

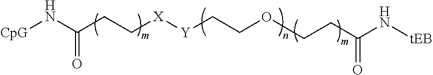

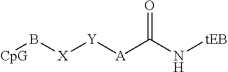

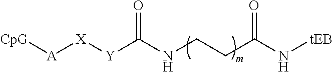

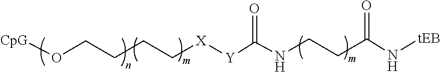

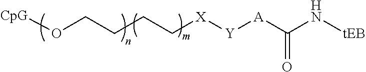

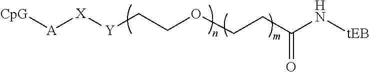

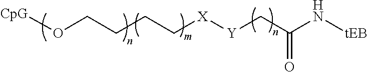

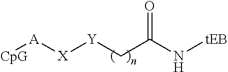

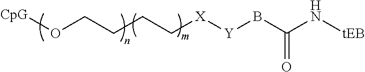

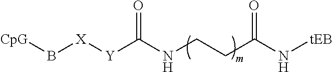

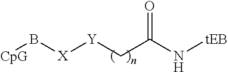

In some embodiments, the chemical conjugates can have structures satisfying any of the formulas illustrated below in Table 2.

TABLE 2

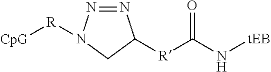

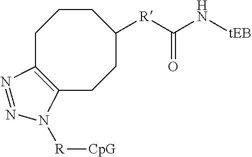

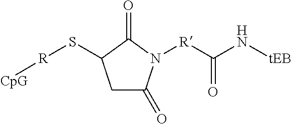

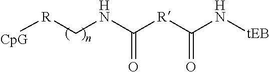

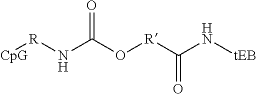

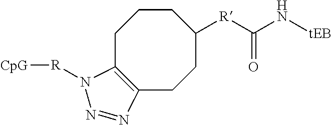

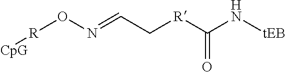

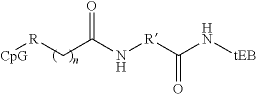

In some embodiments, the chemical conjugates can have the structures illustrated below in Table 3, wherein each n is independently is selected from an integer that provides an R linker length of greater than 0 nm to 200 nm and/or an R′ linker length of greater than 0 nm to 200 nm; and each m independently is selected from 0 or 1. In some embodiments, n ranges from 1 to 600, such as from 1 to 300, or 1 to 150, or 1 to 100, or 1 to 50.

TABLE 3

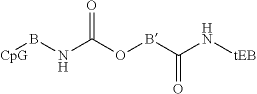

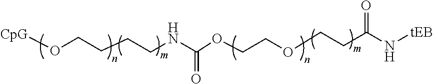

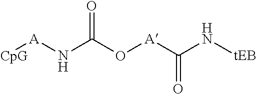

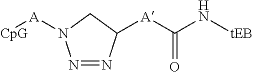

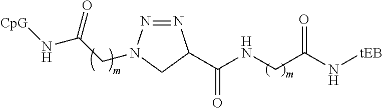

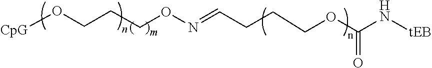

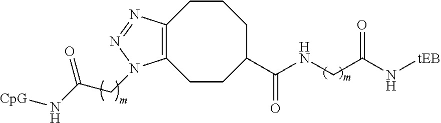

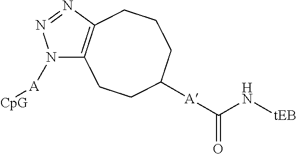

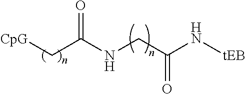

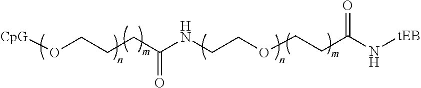

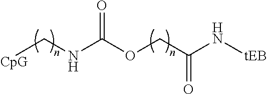

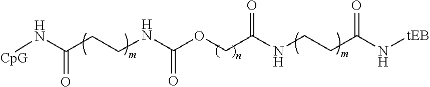

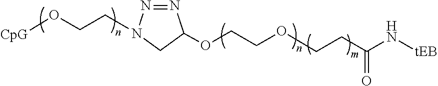

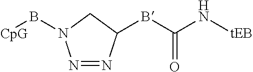

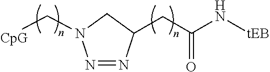

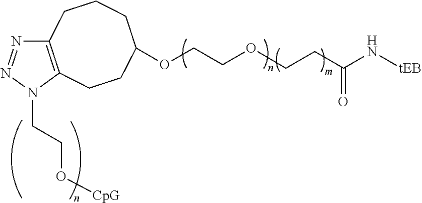

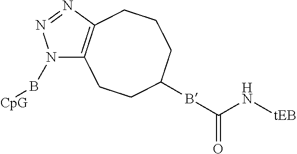

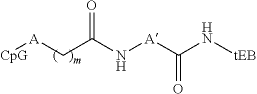

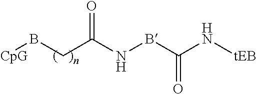

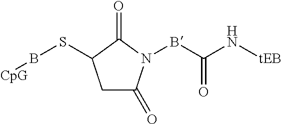

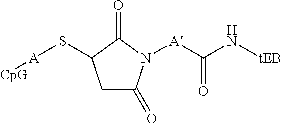

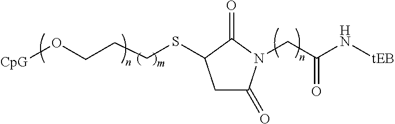

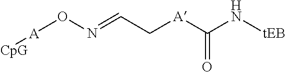

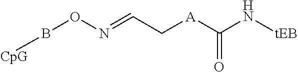

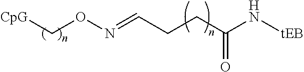

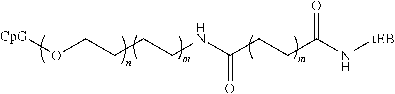

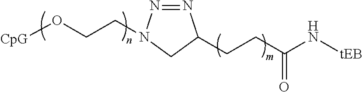

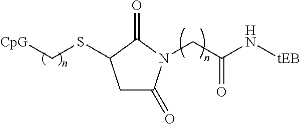

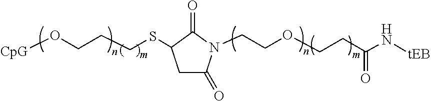

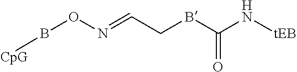

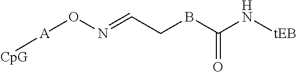

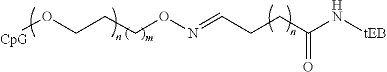

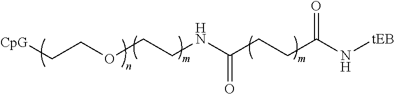

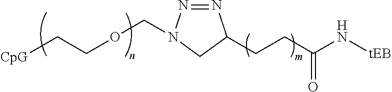

Particular disclosed chemical conjugates can have any of the structures shown in Table 4.

TABLE 4

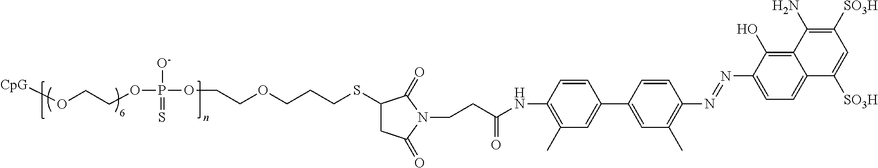

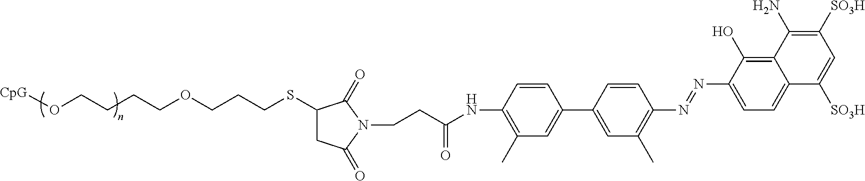

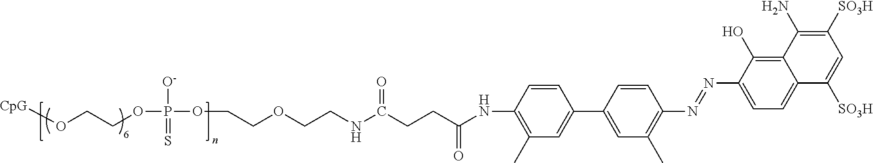

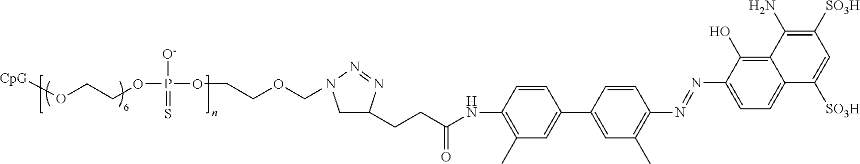

In a particularly preferred embodiment of Formula I, the chemical conjugates of Formula I comprises a tEB component having a structure:

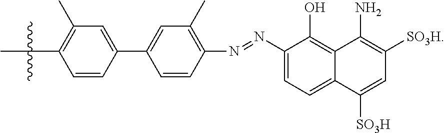 [SMILES: *c1ccc(-c2ccc(/N=N/c3ccc4c(S(=O)(=O)O)cc(S(=O)(=O)O)c(N)c4c3O)c(C)c2)cc1C]

The chemical conjugates described herein can be made using chemical cross-coupling reactions. In particular disclosed embodiments, the methods of making the chemical conjugates can comprise combining a truncated Evans Blue compound with an R′ linker that is then coupled to a Y group, which may or may not be coupled to an X group. In some embodiments, the truncated Evans Blue compound can be coupled to an R′ linker terminated with a carboxylic acid group using peptide coupling reagents, such as, but not limited to, 2-(7-aza-1H-benzotriazol-1-yl)-N,N,N′,N′-tetramethylaminium hexafluorophosphate, 2-(1H-benzotriazol-1-yl)-N,N,N′,N′-hexafluorophosphate, 2-(6-chloro-1H-benzotriazol-1-yl)-N,N,N′,N′-tetramethylaminium hexafluorophosphate, 1-hydroxybenzotriazole, dicyclohexylcarbodiimide, diisopropylcarbodiimide, N-(3-dimethylaminopropyl)-N′-ethylcarbodiimide HCl, benzotriazol-1-yloxy-tris(dimethylamino)-phosphonium hexafluorophosphate, benzotriazol-1-yloxy-tripyrrolidino-phosphonium hexafluorophosphate, bromo-tripyrrolidino-phosphonium hexafluorophosphate, or combinations thereof. In particular disclosed embodiments, R′ is then coupled to a Y group using substitution reactions, peptide coupling reactions, and/or cross-coupling reactions known in the art. X, before or after coupling to Y, can be coupled to an R group using similar peptide coupling conditions described above for coupling R′ and the truncated Evans Blue compound. The R group can then be coupled to a phosphorothioate, a PEG-phosphorothioate, or a polymeric PEG-phosphorothioate, which is (or can be) coupled to the CpG component. In other embodiments, the R group can be coupled directly to the CpG component. Any of the above mentioned method steps can be performed sequentially or substantially simultaneously and in any order. In some embodiments, the methods can involve the steps illustrated below in Scheme 1.

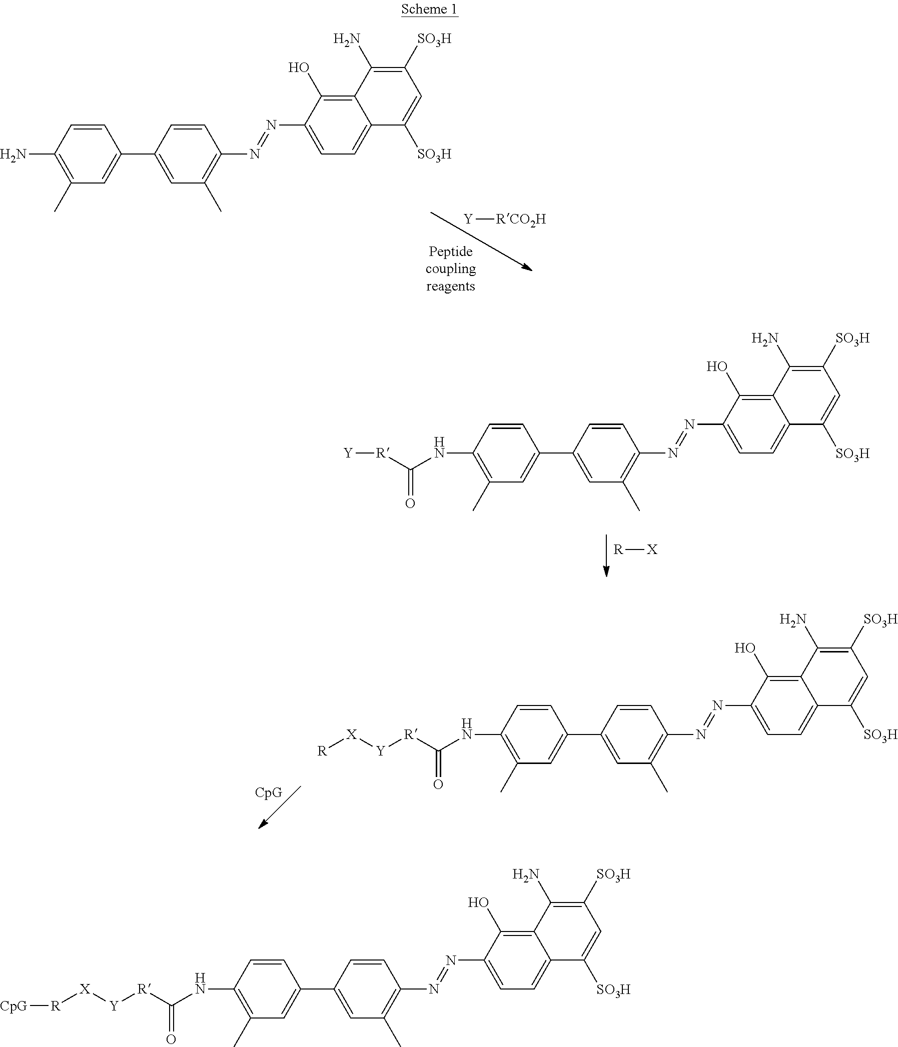

Representative embodiments of methods of making the chemical conjugates described herein are illustrated in Scheme 2.

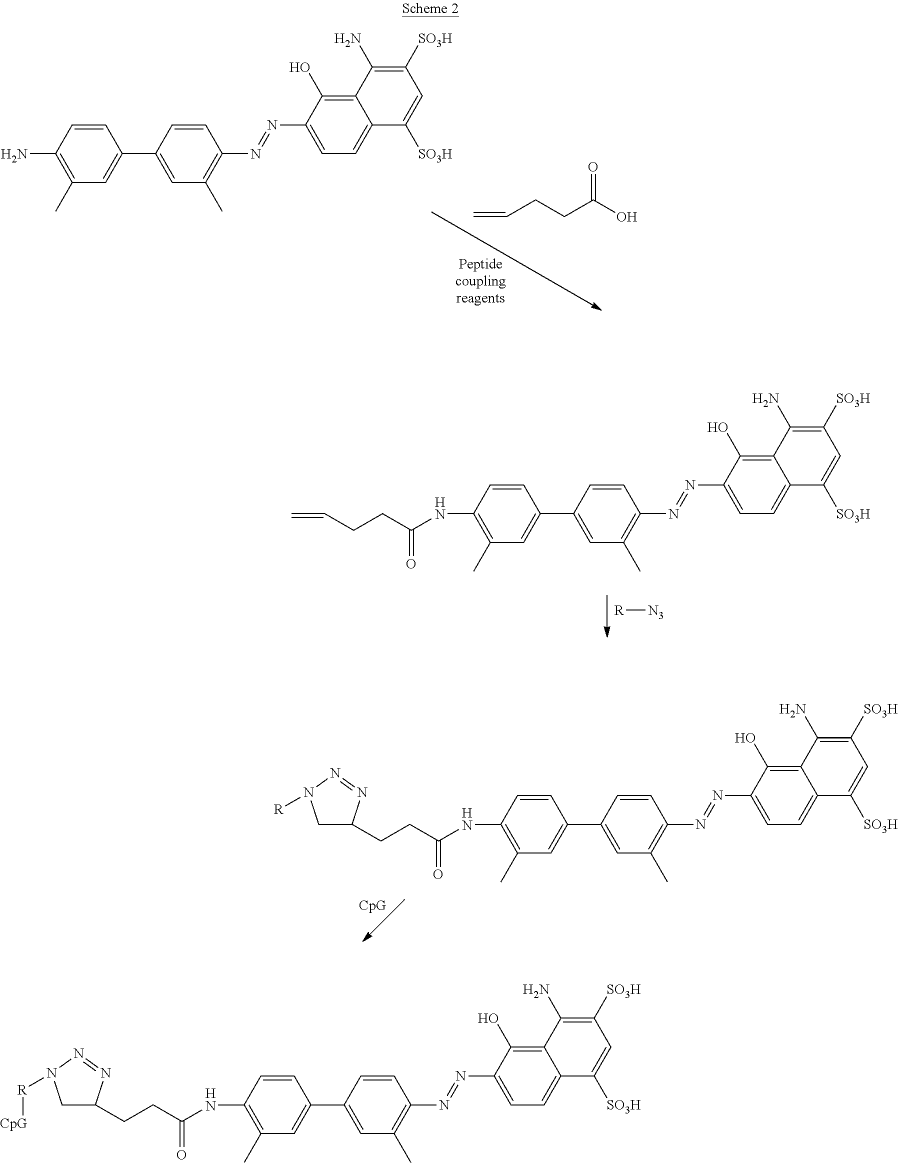

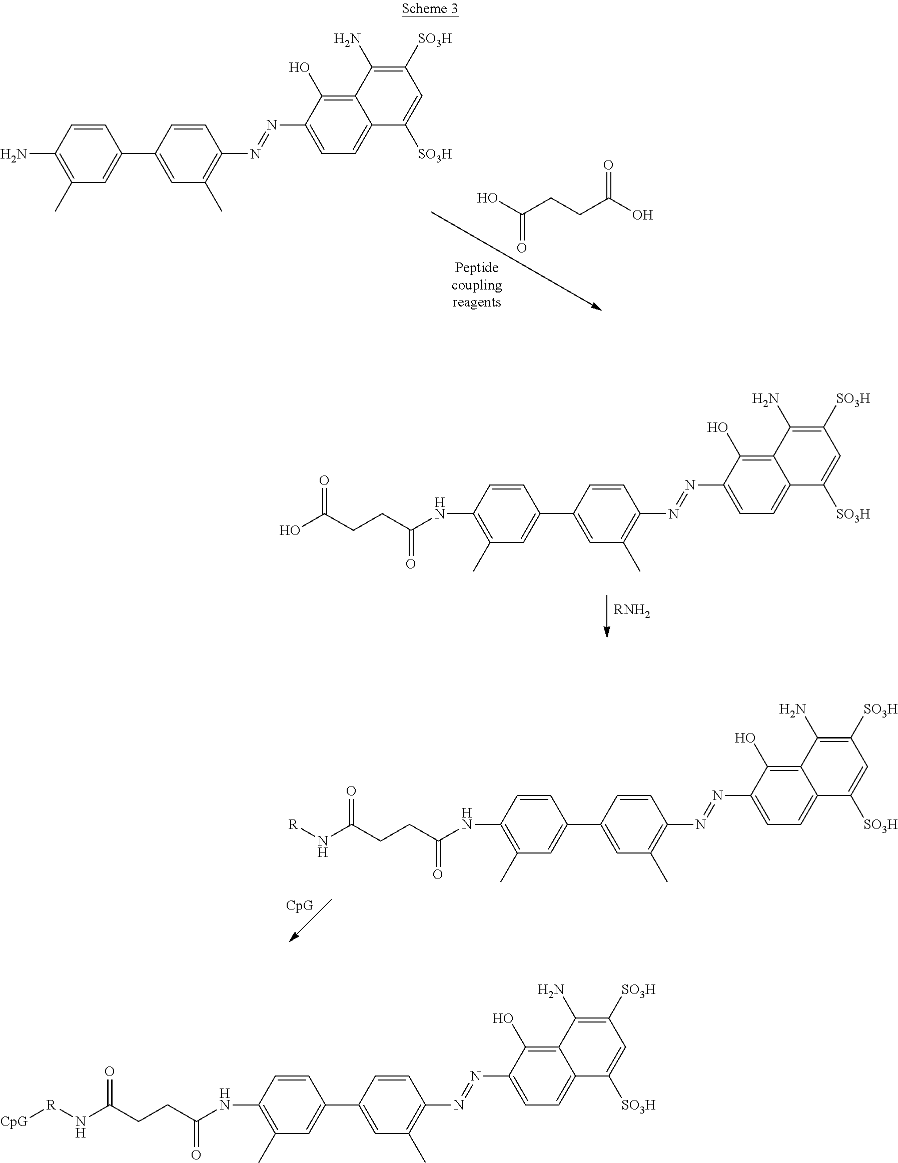

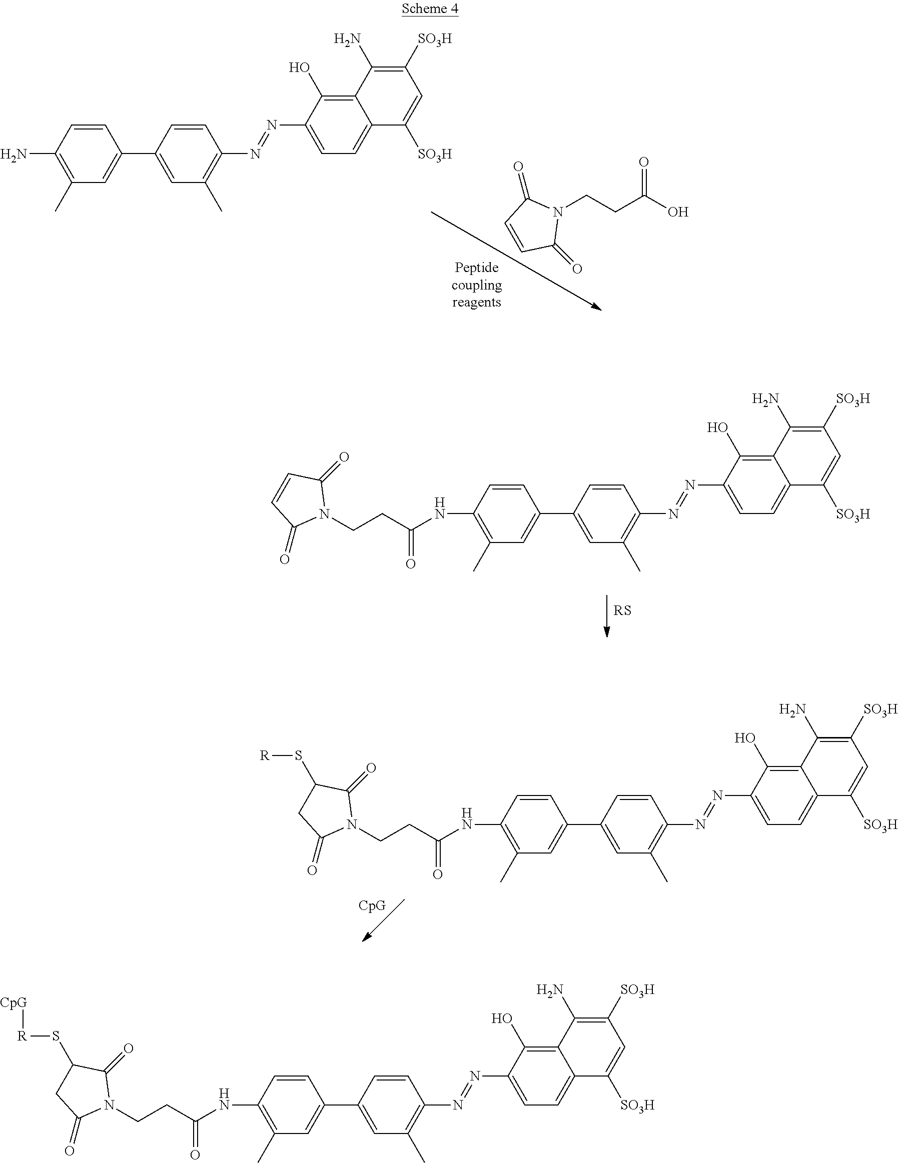

In an exemplary embodiment, a truncated Evans Blue compound can be coupled to an amide linker, which in turn is coupled to a maleimide compound to provide an intermediate Chemical Conjugate I, illustrated below. This intermediate Chemical Conjugate I can then be coupled to a sulfur moiety as illustrated in Chemical Conjugate II, below. The sulfur moiety may be further bound to an R group or it can be coupled to an R group separately. The R group can then be coupled to the CpG group using techniques known to those of ordinary skill in the art, such as peptide couplings, reagents suitable to couple a phosphorothioate to an R linker groups described herein.

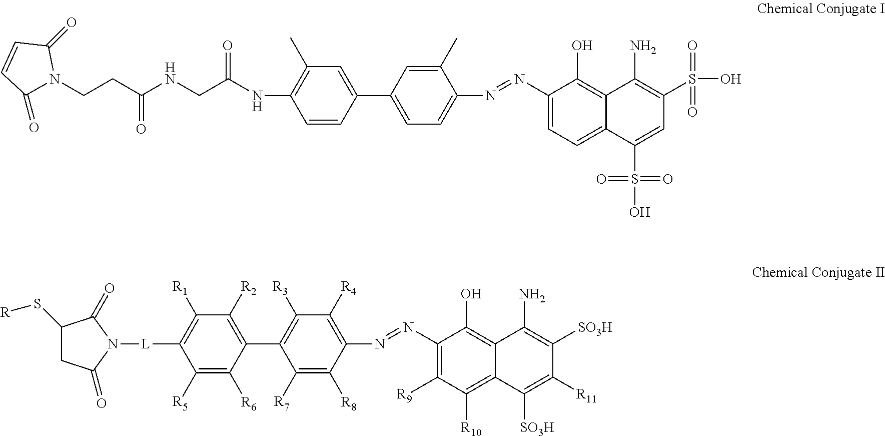

### CpG ODN

The unmethylated CpG motifs present in bacterial DNA stimulate cells that express Toll-like receptor 9 (TLR9). This interaction triggers a short-lived innate immune response characterized by the production of pro-inflammatory cytokines and chemokines (Krieg et al., Nature 374:546-548, 1995; Klinman Nat.Rev.Immunol. 2004, 4 (4):249-258). Microarray identification of the genes triggered by CpG ODN showed that the inflammatory response peaked on day 1, but that >96% of these genes were suppressed by a counter-regulatory process that peaked on day 5 (Klaschik et al, J Leukocyte Biol 85 (5):788-795, 2009; Klaschik et al., Mol. Immunol. 47 (6):1317-1324, 2010). Any immunostimulatory CpD ODN can be used in the chemical conjugates, immunomodulatory compositions and methods disclosed herein, such as K-type ODN, D-type ODN or C-type CpG ODN. Combinations of K-type ODN, D-type ODN or C-type ODNs are also of use. Thus, two K-type CpG ODN, three K-type CpG ODN, two D-type CpG ODN, three D-type CpG ODN, two C-type CpG ODN, or three C-type CpG ODN can be used in the methods disclosed herein. Each of these ODNs can be separately used to form a chemical conjugate as described herein, and multiple chemical conjugates can be administered to a subject, or multiple ODNs can be conjugated to form a single chemical conjugate.

### K-Type CpG ODN

The present methods can include administering a therapeutically effective amount of a K-type CpG oligodeoxynucleotide (ODN). A CpG ODN is an ODN including a CpG motif, wherein the pyrimdine ring of the cytosine is unmethylated. Two types of CpG ODNs have been identified: K-type and D-type ODNs. In several embodiments, the CpG ODN is at most 100 nucleotides or at most 80 nucleotides in length. In other embodiments the CpG ODN is in the range of about 8 to 30 nucleotides in length. In another embodiment, the CpG ODN is at least 10 nucleotides in length, such as about 10 to about 30 nucleotides in length.

K-type CpG ODN of use that are disclosed, for example, in published PCT Application No. WO 98/18810A1 (K-type), which is incorporated by reference herein in its entirety. In some embodiments, only K-type CpG ODNs (or combinations of K-type ODNs) are used in the methods disclosed herein. Thus, in several embodiments, the methods do not include the use of D-type ODNs.

Combinations of K-type CpG ODNs are of use, such as the use of at least two, at least three, at least four, at least five, at least six at least seven, at least eight or at least ten ODNs, each with a different nucleic acid sequence. In several embodiments, two, three, four, five or six K-type CpG ODNs, each with a different nucleic acid sequence, are utilized in the methods.

A single K ODN can be used in the methods disclosed herein, or mixtures of K ODN can also be used in the methods disclosed herein. Specific combinations of ODNs are disclosed, for example, in U.S. patent application Ser. No. 10/194,035, which is incorporated herein by reference.

In several embodiments, a K-type CpG ODN or a mixture of K-type CpG ODNs are utilized. Briefly, the K-type nucleic acid sequences useful in the methods disclosed herein are represented by the formula:

5′-N₁DCGYN₂-3′

wherein at least one nucleotide separates consecutive CpGs; D is adenine, guanine, or thymidine; Y is cytosine or thymine, N is any nucleotide and N₁+N₂ is from about 0-26 bases. In one embodiment, N₁ and N₂ do not contain a CCGG quadmer or more than one CGG trimer; and the nucleic acid sequence is from about 8-30 bases in length, such as about 10 to 30 nucleotides in length. However, nucleic acids of any size (even many kb long) can be used in the methods disclosed herein if CpGs are present. In one embodiment, synthetic oligonucleotides of use do not include a CCGG quadmer or more than one CCG or CGG trimer at or near the 5′ or 3′ terminals and/or the consensus mitogenic CpG motif is not a palindrome. A “palindromic sequence” or “palindrome” means an inverted repeat (i.e., a sequence such as ABCDEE′D′C′B′A′, in which A and A′ are bases capable of forming the usual Watson-Crick base pairs).

In another embodiment, the methods include the use of an ODN which contains a CpG motif represented by the formula:

5′-N₁RDCGYTN₂-3′

wherein at least one nucleotide separates consecutive CpGs; RD is selected from the group consisting of GpT, GpG, GpA, ApT and ApA; YT is selected from the group consisting of TpT or CpT; N is any nucleotide and N₁+N₂ is from about 0-26 bases, such that the ODN is about 8 to 30 nucleotides in length.

In several embodiments, the methods disclosed herein include the use of an effective amount of at least one K-type CpG ODN, wherein the K-type CpG ODN includes an unmethylated CpG motif that has a sequence represented by the formula:

(SEQ ID NO: 1)

5′ N₁N₂N₃D-CpG-WN₄N₅N₆ 3′

wherein the central CpG motif is unmethylated, D is T, G or A, W is A or T, and N₁, N₂, N₃, N₄, N₅, and N₆ are any nucleotides. In one embodiment, D is a T. The K ODN(s) can be 10 to 30 nucleotides in length. A K-type CpG ODN can include multiple CpG motifs. In some embodiments, at least one nucleotide separates consecutive CpGs; N₃D is selected from the group consisting of GpT, GpG, GpA, ApT and ApA; WN₄ is selected from the group consisting of TpT or CpT; N is any nucleotide and N₁+N₂ is from about 0-26 bases

In one embodiment, N₁, and N₂ do not contain a CCGG quadmer or more than one CCG or CGG trimer. CpG ODN are also in the range of 8 to 30 bases in length, but may be of any size (even many kb long) if sufficient motifs are present. In several examples, the CpG ODN is 10 to 20 nucleotides in length, such as 12 to 18 nucleotides in length. In one embodiment, synthetic ODNs of this formula do not include a CCGG quadmer or more than one CCG or CGG trimer at or near the 5′ and/or 3′ terminals and/or the consensus CpG motif is not a palindrome. Other CpG ODNs can be assayed for efficacy using methods described herein. It should be noted that exemplary K-type CpG ODNs are known in the art, and have been fully described, for example in PCT Publication No. WO 98/18810A1, which is incorporated herein by reference.

Exemplary K-type CpG ODN are listed below:

TABLE 1
K ODN

| | | |
|---|---|---|
| CpG 1826 | TCCATGACGTTCCTGACGTT | (SEQ ID NO: 2) |
| CpG 7909 | TCGTCGTTTTGTCGTTTTGTCGTT | (SEQ ID NO: 3) |
| CpG10103 | TCGTCGTTTTTCGGTCGTTTT | (SEQ ID NO: 4) |
| K X | ATAATCGACGTTCAAGCAAG | (SEQ ID NO: 5) |
| K22 | CTCGAGCGTTCTC | (SEQ ID NO: 6) |
| K21 | TCTCGAGCGTTCTC | (SEQ ID NO: 7) |
| K82 | ACTCTGGAGCGTTCTC | (SEQ ID NO: 8) |
| K30 | TGCAGCGTTCTC | (SEQ ID NO: 9) |
| k31 | TCGAGGCTTCTC | (SEQ ID NO: 10) |
| K39 | GTCGGCGTTGAC | (SEQ ID NO: 11) |
| K16 | TCGACTCTCGAGCGTTCTC | (SEQ ID NO: 12) |
| K3 | ATCGACTCTCGAGCGTTCTC | (SEQ ID NO: 13) |
| k23 | TCGAGCGTTCTC | (SEQ ID NO: 14) |
| K40 | GTCGGCGTCGAC | (SEQ ID NO: 15) |
| K34 | GTCGACGTTGAC | (SEQ ID NO: 16) |
| K83 | ACTCTCGAGGGTTCTC | (SEQ ID NO: 17) |
| K19 | ACTCTCGAGCGTTCTC | (SEQ ID NO: 18) |
| K73 | GTCGTCGATGAC | (SEQ ID NO: 19) |
| K46 | GTCGACGCTGAC | (SEQ ID NO: 20) |
| K47 | GTCGACGTCGAC | (SEQ ID NO: 21) |
| K72 | GTCATCGATGCA | (SEQ ID NO: 22) |
| K37 | GTCAGCGTCGAC | (SEQ ID NO: 23) |
| k25 | TCGAGCGTTCT | (SEQ ID NO: 24) |
| K82 | ACTCTGGAGCGTTCTC | (SEQ ID NO: 25) |
| K83 | ACTCTCGAGGGTTCTC | (SEQ ID NO: 26) |
| K84 | ACTCTCGAGCGTTCTA | (SEQ ID NO: 27) |
| K85 | CATCTCGAGCGTTCTC | (SEQ ID NO: 28) |
| K89 | ACTCTTTCGTTCTC | (SEQ ID NO: 29) |
| K109 | TCGAGCGTTCT | (SEQ ID NO: 30) |
| K123 | TCGTTCGTTCTC | (SEQ ID NO: 31) |
| K1555 | GCTAGACGTTAGCGT | (SEQ ID NO: 32) |
| K110 | TCGAGGCTTCTC | (SEQ ID NO: 33) |

Exemplary Control ODNs are:

GC

(SEQ ID NO: 34)

TCCATGAGCTTCCTGAGCTT
K1612

(SEQ ID NO: 35)

TAGAGCTTAGCTTGC
C163

(SEQ ID NO: 36)

TTGAGTGTTCTC

As noted above, combinations of K-type CpG ODN can also be used. Exemplary combinations include 1) K3, K19, K110; 2) K19, K23, K123; K3, 3) K110, K123; 4) K3, K23, K123; 5) K3, K19, K123; and 6) K19, K110, K123. Additional exemplary combinations include at least two different K-type CpG ODNS, wherein one of the K-type CpG ODNs is K1555, and/or wherein one of the K-type CpG ODNs is K3.

### D-Type CpG ODN

In another embodiment, the methods include administering an effective amount a “D type” CpG ODN (see Verthelyi et al, J. Immunol. 166:2372, 2001; U.S. Pat. No. 7,960,356, both of which are herein incorporated by reference in their entirety). D type ODNs differ both in structure and activity from K type ODNs. For example, D ODNs stimulate the release of cytokines, such as IP-10 and IFN-α, by monocytes and/or plasmacitoid dendritic cells and the release or production of IFN-γ by natural killer (NK) cells. The stimulation of NK cells by D ODNs can be either direct or indirect.

With regard to structure, in one embodiment, a CpG motif for a D type oligonucleotides can have the structure:

5′ RY-CpG-RY 3′

wherein the central CpG motif is unmethylated, R is A or G (a purine), and Y is C or T (a pyrimidine). D-type oligonucleotides include an unmethylated CpG dinucleotide. Inversion, replacement or methylation of the CpG reduces or abrogates the activity of the D oligonucleotide.

In one embodiment, a D type ODN is at least about 16 nucleotides in length and includes a sequence represented by:

(SEQ ID NO: 37)

5′ X₁X₂X₃ Pu₁ Py₂ CpG Pu₃ Py₄ X₄X₅X₆(W)_{M} (G)_{N}-3′

wherein the central CpG motif is unmethylated, Pu is a purine nucleotide, Py is a pyrimidine nucleotide, X and W are any nucleotide, M is any integer from 0 to 10, and N is any integer from 4 to 8.

The region Pu₁ Py₂ CpG Pu₃ Py₄ is termed the CpG motif. The region X₁X₂X₃ is termed the 5′ flanking region, and the region X₄X₅X₆ is termed the 3′ flanking region. If nucleotides are included 5′ of X₁X₂X₃ in the D ODN these nucleotides are termed the 5′ far flanking region. Nucleotides 3′ of X₄X₅X₆ in the D ODN are termed the 3′ far flanking region.

In one specific non-limiting example, Py₂ is a cytosine. In another specific, non-limiting example, Pu₃ is a guanidine. In yet another specific, non-limiting example, Py₂ is a thymidine and Pu₃ is an adenine. In a further specific, non-limiting example, Pu₁ is an adenine and Py₂ is a tyrosine. In another specific, non-limiting example, Pu₃ is an adenine and Py₄ is a tyrosine.

In one specific not limiting example, N is from about 4 to about 8. In another specific, non-limiting example, N is about 6. In further specific non-limiting examples, N is 4, 5, 7 or 8.

D-type CpG ODNs can include modified nucleotides. These modified nucleotides can be included to increase the stability of a D-type oligonucleotide. Without being bound by theory, because phosphorothioate-modified nucleotides confer resistance to exonuclease digestion, ODN are “stabilized” by incorporating phosphorothioate-modified nucleotides. In one embodiment, the CpG dinucleotide motif and its immediate flanking regions include phosphodiester rather than phosphorothioate nucleotides. In one specific non-limiting example, the sequence Pu₁ Py₂ CpG Pu₃ Py₄ includes phosphodiester bases. In another specific, non-limiting example, all of the bases in the sequence Pu₁ Py₂ CpG Pu₃ Py₄ are phosphodiester bases. In yet another specific, non-limiting example, X₁X₂X₃ and X₄X₅X₆(W)_{M} (G)_{N} include phosphodiester bases. In yet another specific, non-limiting example, X₁X₂X₃ Pu₁ Py₂ CpG Pu₃ Py₄ X₄X₅X₆(W)_{M} (G)_{N}(SEQ ID NO: 37) includes phosphodiester bases. In further non-limiting examples the sequence X₁X₂X₃ includes at most one or at most two phosphothioate bases and/or the sequence X₄X₅X₆ includes at most one or at most two phosphotioate bases. In additional non-limiting examples, X₄X₅X₆(W)_{M} (G)_{N} includes at least 1, at least 2, at least 3, at least 4, or at least 5 phosphothioate bases. Thus, a D type ODN can be a phosphorothioate/phosphodiester chimera.

As disclosed herein, any suitable modification can be used in the present invention to render the ODN resistant to degradation in vivo (e.g., via an exo- or endo-nuclease). In one specific, non-limiting example, a modification that renders the ODN less susceptible to degradation is the inclusion of nontraditional bases such as inosine and quesine, as well as acetyl-, thio- and similarly modified forms of adenine, cytidine, guanine, thymine, and uridine. Other modified nucleotides include nonionic DNA analogs, such as alkyl or aryl phosphonates (i.e., the charged phosphonate oxygen is replaced with an alkyl or aryl group, as set forth in U.S. Pat. No. 4,469,863), phosphodiesters and alkylphosphotriesters (i.e., the charged oxygen moiety is alkylated, as set forth in U.S. Pat. No. 5,023,243 and European Patent No. 0 092 574). Oligonucleotides containing a diol, such as tetraethyleneglycol or hexaethyleneglycol, at either or both termini, have also been shown to be more resistant to degradation. The D type ODNs can also be modified to contain a secondary structure (e.g., stem loop structure). Without being bound by theory, it is believed that incorporation of a stem loop structure renders an ODN more effective.

In a further embodiment, Pu₁ Py₂ and Pu₃ Py₄ are self-complementary. In another embodiment, X₁X₂X₃ and X₄X₅X₆ are self-complementary. In yet another embodiment X₁X₂X₃ Pu₁ Py₂ and Pu₃ Py₄ X₄X₅X₆ are self-complementary.

Specific non-limiting examples of a D type oligonucleotide wherein Pu₁ Py₂ and Pu₃ Py₄ are self-complementary include, but are not limited to, ATCGAT, ACCGGT, ATCGAC, ACCGAT, GTCGAC, or GCCGGC. Without being bound by theory, the self-complementary base sequences can help to form a stem-loop structure with the CpG dinucleotide at the apex to facilitate immunostimulatory functions. Thus, in one specific, non-limiting example, D type oligonucleotides wherein Pu₁ Py₂ and Pu₃ Py₄ are self-complementary induce higher levels of IFN-γ production from a cell of the immune system (see below). The self-complementary need not be limited to Pu₁ Py₂ and Pu₃ Py₄. Thus, in another embodiment, additional bases on each side of the three bases on each side of the CpG-containing hexamer form a self-complementary sequence (see above).

Exemplary D type ODNs are well known in the art. Specific non-limiting examples include, but are not limited to:

(SEQ ID NO: 38)

5′XXTGCATCGATGCAGGGGGG 3′

(SEQ ID NO: 39)

5′XXTGCACCGGTGCAGGGGGG3′,

(SEQ ID NO: 40)

5′XXTGCGTCGACGCAGGGGGG3′,

(SEQ ID NO: 41)

5′XXTGCGTCGATGCAGGGGGG3′,

(SEQ ID NO: 42)

5′XXTGCGCCGGCGCAGGGGGG3′,

(SEQ ID NO: 43)

5′XXTGCGCCGATGCAGGGGGG3′,

(SEQ ID NO: 44)

5′XXTGCATCGACGCAGGGGGG3′,

(SEQ ID NO: 45)

5′XXTGCGTCGGTGCAGGGGGG3′,

wherein X any base, or is no base at all. In one specific, non-limiting example, X is a G.
Further examples include (underlined bases are phosphodiester):

TABLE 2
D-type CpG ODN

| | | |
|---|---|---|
| D104 | GGTGCATCGATGCAGGGGGG | (SEQ ID NO: 46) |
| D19 | GGTGCATCGATGCAGGGGGG | (SEQ ID NO: 47) |
| D29 | GGTGCACCGGTGCAGGGGGG | (SEQ ID NO: 48) |
| D35 | GGTGCATCGATGCAGGGGGG | (SEQ ID NO: 49) |
| D28 | GGTGCGTCGATGCAGGGGGG | (SEQ ID NO: 50) |
| D106 | GGTGTGTCGATGCAGGGGGG | (SEQ ID NO: 51) |
| D116 | TGCATCGATGCAGGGGGG | (SEQ ID NO: 52) |
| D113 | GGTGCATCGATACAGGGGGG | (SEQ ID NO: 53) |
| D34 | GGTGCATCGATGCAGGGGGG | (SEQ ID NO: 54) |
| D102 | GGTGCATCGTTGCAGGGGGG | (SEQ ID NO: 55) |
| D32 | GGTGCGTCGACGCAGGGGGG | (SEQ ID NO: 56) |
| D117 | GGTCGATCGATGCACGGGGG | (SEQ ID NO: 57) |
| D37 | GGTGCATCGATGCAGGGGGG | (SEQ ID NO: 58) |
| D25 | GGTGCATCGATGCAGGGGGG | (SEQ ID NO: 58) |
| D30 | GGTGCATCGACGCAGGGGGG | (SEQ ID NO: 59) |
| d120 | GGTGCATCGATAGGCGGGGG | (SEQ ID NO: 60) |
| D27 | GGTGCACCGATGCAGGGGGG | (SEQ ID NO: 61) |
| d119 | CCTGCATCGATGCAGGGGGG | (SEQ ID NO: 62) |
| D142 | GGTATATCGATATAGGGGGG | (SEQ ID NO: 63) |
| d143 | GGTGGATCGATCCAGGGGGG | (SEQ ID NO: 64) |

Combinations of these ODNs are also of use. In some embodiments, only D-type CpG ODNs (or combinations of D-type ODNs) are used in the methods disclosed herein. Thus, in several embodiments, the methods do not include the use of K-type ODNs. Combinations of D-type CpG ODNs are of use, such as the use of at least two, at least three, at least four, at least five, at least six at least seven, at least eight or at least ten ODNs, each with a different nucleic acid sequence. In several embodiments, two, three, four, five or six D-type CpG ODN are utilized. An exemplary combination of D ODNs of use is D19, D29 and D35. Another exemplary combination is D19, D29 and D35. However, other combinations of ODN can be used, such as any two or three ODN from the Table above.

### C-type CpG ODN

C-type ODNs also can be utilized in the methods disclosed herein. Typically, C class ODNs have a TCGTCG motif at the 5′ end and have a CpG motif imbedded in a palindromic sequence. M362 is an exemplary C-type CpG ODN that contains a 5′-end ‘TCGTCG-motif’ and a ‘GTCGTT-motif’. C-type ODNs resemble K-type as they are composed entirely of phosphorothioate nucleotides, but resemble D-type in containing palindromic CpG motifs. This class of ODNs stimulates B cells to secrete IL-6 and pDCs to produce IFN-α (see Hartmann et al., Eur. J. Immunol. 33: 1633-41, 2003, incorporated herein by reference). A palindromic sequence of at least 8 nucleotides increases activity, for example a palindrome of at least 12, such as 14, 16, 18 or 20 nucleotides, increases activity.

In some embodiments, the CpG-C ODNs include one to two TCG trinucleotides at or close to the 5′ end of the ODN and a palindromic region of at least 10-12 bases, which contains at least two additional CG dinucleotides preferably spaced zero to three bases apart. The CG dinucleotides in the palindrome are preferably spaced 1, 2, or 3 nucleotides apart, although sequences with four nucleotide spacings retained low levels of IFN-α-inducing activity (see Marshall et al., J. Leukocyte Biol. 73: 781-792, 2003, incorporated herein by reference). C-type ODNs are present in both early and late endosomes, and thus express properties in common with both K- and D-type CpG ODNs. C-type CpG ODNs include ODN2216

SEQ ID NO: 65

(ggGGGACGA:TCGTCgggggg,,

wherein the bases shown in capital letters are phosphodiester, and those in lower case are phosphorothioate) ODN M362

SEQ ID NO: 66)

(tcgtcgtcgttcgaacgacgttgat,,

ODN 1668 (tccatgacgttcctgatgct, SEQ ID NO: 67), and ODN2395 (tcgtcgttttcggcgcgcgccg, SEQ ID NO: 68), which are available from Invivogen and C274 (TCGTCGAACGTTCGAGATGAT, SEQ ID NO: 69, wherein all the bases are phophorothioate except the last base), which is available from Novusbio. C-type ODNs also can be modified to be resistant to degradation, as disclosed herein.

### Immunomodulatory Compositions

Chemical conjugates disclosed herein can be administered as the neat chemical, but are preferably administered as a pharmaceutical composition, such as an immunomodulatory composition. Accordingly, this disclosure encompasses immunomodulatory compositions comprising a chemical conjugate or pharmaceutically acceptable salt of a chemical conjugate, such as a chemical conjugate of any of Formulas I, III-VII, the formulas provided by Tables 1-3, or the particular chemical conjugates provided by Table 4, together with at least one pharmaceutically acceptable carrier. The immunomodulatory composition may contain a chemical conjugate or salt thereof as the only active agent, but can include one or more additional active agents. In certain embodiments the immunomodulatory composition is in a dosage form that contains from about 0.1 mg to about 2000 mg, from about 10 mg to about 1000 mg, from about 100 mg to about 800 mg, or from about 200 mg to about 600 mg of a chemical conjugate and optionally from about 0.1 mg to about 2000 mg, from about 10 mg to about 1000 mg, from about 100 mg to about 800 mg, or from about 200 mg to about 600 mg of an additional active agent in a unit dosage form. The immunomodulatory composition may also include a molar ratio of a compound, such as a chemical conjugate, and an additional active agent. For example the immunomodulatory composition may contain a molar ratio of about 0.5:1, about 1:1, about 2:1, about 3:1 or from about 1.5:1 to about 4:1 of an additional active agent to a chemical conjugate.

Chemical conjugates disclosed herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The immunomodulatory composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the chemical conjugate is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in an immunomodulatory composition, which do not substantially interfere with the activity of the chemical conjugate of the present disclosure.

The immunomodulatory compositions/combinations can be formulated for oral administration. These immunomodulatory compositions contain between 0.1 and 99 weight % (wt. %) of a chemical conjugate and usually at least about 5 wt. % of a chemical conjugate. Some embodiments contain from about 25 wt % to about 50 wt % or from about 5 wt % to about 75 wt % of the chemical conjugate. Additional methods and compositions are disclosed below.

### Methods for Inducing an Immune Response and Treatment

The disclosed chemical conjugates are of use to induce an immune response to an antigen, such as a protective or therapeutic immune response. The immune response can be to any antigen of interest, including antigens from pathogens and tumor antigens. Thus, the disclosed chemical conjugates are of use to treat or prevent disease, including but not limited to diseases causes by pathogens and tumors.

In some embodiments, the disclosed immunomodulatory compositions or chemical conjugates are administered with an antigen of interest. The antigen of interest can be in a vaccine, such as a subunit vaccine, a heat killed vaccine, or an attenuated vaccine. The disclosed chemical conjugates, and immunomodulatory compositions comprising the chemical conjugates, are useful for treatment of diseases such as those caused by an infection with a pathogen or a tumor. The infection can be a persistent infection or an acute infection.

In one embodiment, the subject is a mammal, such a human. As will be understood by one skilled in the art, the methods also encompass treating non-human subjects. As will be appreciated by one skilled in the art, the methods can be used for veterinary applications such as to treat horses and livestock, e.g. cattle, sheep, cows, goats, swine and the like, and pets (companion animals) such as dogs and cats. For diagnostic or research applications, a wide variety of mammals will be suitable subjects including rodents (e.g. mice, rats, hamsters), rabbits, primates, and swine such as inbred pigs and the like.

For prophylactic and therapeutic purposes, an immunomodulatory composition or chemical conjugate disclosed herein can be administered to the subject, optionally in combination with an antigen or a vaccine, in a single bolus delivery, via continuous delivery (for example, continuous transdermal, mucosal or intravenous delivery) over an extended time period, or in a repeated administration protocol (for example, by an hourly, daily or weekly, repeated administration protocol). As noted above, vaccine can be, for example, a subunit vaccine, an attenuated vaccine (such as an attenuated viral vaccine), or a heat killed vaccine.

The therapeutically effective dosage of the immunomodulatory composition/chemical conjugate and optionally the antigen and/or vaccine can be provided as repeated doses within a prolonged prophylaxis or treatment regimen that will yield clinically significant results to alleviate one or more symptoms or detectable conditions associated with a targeted disease or condition as set forth herein. Prime boost strategies are appropriate.

Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the chemical conjugate (for example, amounts that are effective to elicit a desired immune response or alleviate one or more symptoms of a targeted disease). In alternative embodiments, an effective amount or effective dose of the compound, optionally with an antigen and/or a vaccine, may simply inhibit or enhance one or more selected biological activities correlated with a disease or condition, as set forth herein, for either therapeutic or diagnostic purposes.

A therapeutically effective amount of an immunomodulatory composition (or chemical conjugate) is preferably an amount sufficient to reduce or ameliorate the symptoms of a disease or condition. In the case of a tumor for example, a therapeutically effective amount may be an amount sufficient to reduce or ameliorate a symptom of the tumor, tumor volume or metastasis. The tumor can be benign or malignant. In the case of an infection with a pathogen, a therapeutically effective amount may be an amount sufficient to reduce or ameliorate a symptom of infection, or to decrease the number of disease causing agent, such as a reduction in viral load or the number of bacteria. The therapeutically effective amount of an immunomodulatory composition (or chemical conjugate) as disclosed herein can be administered to induce a protective immune response, such as to prevent a future infection with a pathogen, such as, but not limited to, a fungus, virus or bacteria.

A therapeutically effective amount of a chemical conjugate or immunomodulatory composition described herein will also provide a sufficient concentration of a when administered to a patient. A sufficient concentration is preferably a concentration of the chemical conjugate in the patient's body necessary to prevent or combat the disorder. Such an amount may be ascertained experimentally, for example by assaying blood concentration of the compound, or theoretically, by calculating bioavailability.

The actual dosage will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the vaccine for eliciting the desired activity or biological response in the subject. Dosage regimens can be adjusted to provide an optimum prophylactic or therapeutic response. A therapeutically effective amount is also one in which any toxic or detrimental side effects of the chemical conjugate and/or other biologically active agent is outweighed in clinical terms by therapeutically beneficial effects. A non-limiting range for a therapeutically effective amount of the chemical conjugate and/or other biologically active agent, such as an antigen or vaccine, within the methods and formulations of the disclosure is about 0.001 mg/kg body weight to about 10 mg/kg body weight, such as 0.01 mg/kg body weight to about 10 mg/kg body, for example about 0.001 mg/kg, about 0.002 mg/kg, about 0.003 mg/kg, about 0.004 mg/kg, 0.005 mg/kg, about 0.006 mg/kg, about 0.007 mg/kg, about, 0.008 mg/kg, about 0.009 mg/kg, 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. In some embodiments, the dosage includes a set amount of a disclosed compound, such as from about 1-300 μg, for example, a dosage of about 1, 2, 3, 4, 5, 6, 7, 8, 9, 10, 15, 20, 30, 40, 50, 60, 70, 80, 90, 100, 150, 200, 250, or about 300 μg.

In some embodiments, dosage levels of the chemical conjugate are from about 0.001 mg to about 140 mg per kilogram of body weight per day (about 0.5 mg to about 7 g per patient per day). The dosage level can be from about 0.01 mg to about 140 mg per kilogram of body weight per day. Dosage unit forms can contain between from about 1 mg to about 500 mg of each active compound. In certain embodiments 25 mg to 500 mg, or 25 mg to 200 mg of an immunomodulatory are provided daily to a subject. Frequency of dosage may also vary depending on the chemical conjugate used and the particular disease treated. However, for treatment of most diseases and disorders, a dosage regimen of 4 times daily or less can be used and in certain embodiments a dosage regimen of 1 or 2 times daily is used. An exemplary dose is 100 mg per subject, one time per day. Additional doses of use are disclosed, for example, in PCT Publication No. WO2013151771 A1, which is incorporated by reference herein.

It will be understood, however, that the specific dose level for any particular patient will depend upon a variety of factors including the activity of the specific chemical conjugate employed, the age, body weight, general health, sex, diet, time of administration, route of administration, and rate of excretion, drug combination and the severity of the particular disease undergoing therapy.

Upon administration (for example, via injection, aerosol, oral, topical or other route), the immune system of the subject typically responds by producing an immune response, such as, but not limited to, producing antibodies and/or T cells specific for the antigen of interest. Such a response signifies that an immunologically effective dose was delivered. An immunologically effective dosage can be achieved by single or multiple administrations (including, for example, multiple administrations per day), daily, or weekly administrations. For each particular subject, specific dosage regimens can be evaluated and adjusted over time according to the individual need and professional judgment of the person administering or supervising the administration of the vaccine. In some embodiments, the antibody response of a subject administered the immunomodulatory compositions of the disclosure will be determined in the context of evaluating effective dosages/immunization protocols. In most instances it will be sufficient to assess the antibody titer in serum or plasma obtained from the subject. Decisions as to whether to administer booster inoculations and/or to change the amount of the immunomodulatory composition administered to the individual can be at least partially based on the antibody titer level. The antibody titer level can be based on, for example, an immunobinding assay which measures the concentration of antibodies in the serum which bind to a specific antigen, for example, a viral antigen or a tumor antigen.

In some embodiments, when used to prevent or treat an infection, such as a viral infection (such as human immunodeficiency virus (HIV), influenza, cytomegalovirus, hepatitis virus, etc., see below), an antigen of interest from the virus and a chemical conjugate (or immunomodulatory composition) disclosed herein are administered to a subject induces an immune response in the subject that neutralizes the relevant virus. To assess neutralization activity, following immunization of a subject, serum can be collected from the subject at appropriate time points, frozen, and stored for neutralization testing. Methods to assay for neutralization activity are known to the person of ordinary skill in the art and are further described herein, and include, but are not limited to, plaque reduction neutralization (PRNT) assays, microneutralization assays, flow cytometry based assays, single-cycle infection assays. In some embodiments, the serum neutralization activity can be assayed using a panel of pseudoviruses. In other embodiments, to treat a tumor, an antigen of interest from the tumor and a chemical conjugate (or immunomodulatory composition) disclosed herein are administered to a subject, such that a sign or a symptom of the tumor are treated. In some non-limiting examples, tumor volume, tumor mass, or the number or size of metastasis are reduced.

An immunomodulatory composition including one or more of the disclosed chemical conjugates can be used in coordinate (or prime-boost) vaccination protocols or combinatorial formulations. In certain embodiments, novel combinatorial immunomodulatory compositions and coordinate immunization protocols employ separate formulations, each directed toward eliciting an immune response. Separate immunomodulatory compositions that elicit an immune response can be combined in a polyvalent immunomodulatory composition administered to a subject in a single immunization step, or they can be administered separately (in monovalent immunomodulatory compositions) in a coordinate (or prime-boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect. If such monitoring indicates that vaccination is sub-optimal, the subject can be boosted with an additional dose of immunomodulatory composition, and the vaccination parameters can be modified in a fashion expected to potentiate the immune response. Thus, for example, the dose of the disclosed immunomodulatory composition and/or antigen can be increased or the route of administration can be changed.

The immunomodulatory compositions (and chemical compounds) disclosed herein offer the advantage that, due to the relatively strong binding of EB moiety with albumin, the in vivo biodistribution can be controlled such that the chemical conjugates are directed to the lymph nodes. In addition, the structure is such that the EB moiety does not interfere with the immunostimulatory function of the CpG ODN. Thus, an efficient system for inducing an immune response is provided. The immunodulatory compositions (and chemical conjugates) can be used with antigens and/or vaccines from any of the below listed pathogens and tumors. One of skill in the art readily understands that there are additional embodiments, and that the lists below are not exhaustive.

i. Viral Pathogens

Specific examples of viral pathogens include without limitation any one or more of (or any combination of) Arenaviruses (such as Guanarito virus, Lassa virus, Junin virus, Machupo virus and Sabia), Arteriviruses, Roniviruses, Astroviruses, Bunyaviruses (such as Crimean-Congo hemorrhagic fever virus and Hantavirus), Bamaviruses, Birnaviruses, Bomaviruses (such as Boma disease virus), Bromoviruses, Caliciviruses, Chrysoviruses, Coronaviruses (such as Coronavirus and SARS), Cystoviruses, Closteroviruses, Comoviruses, Dicistroviruses, Flaviruses (such as Yellow fever virus, West Nile virus, Hepatitis C virus, and Dengue fever virus), Filoviruses (such as Ebola virus and Marburg virus), Flexiviruses, Hepeviruses (such as Hepatitis E virus), human adenoviruses (such as human adenovirus A-F), human astroviruses, human BK polyomaviruses, human bocaviruses, human coronavirus (such as a human coronavirus HKU1, NL63, and OC43), human enteroviruses (such as human enterovirus A-D), human erythrovirus V9, human foamy viruses, human herpesviruses (such as human herpesvirus 1 (herpes simplex virus type 1), human herpesvirus 2 (herpes simplex virus type 2), human herpesvirus 3 (Varicella zoster virus), human herpesvirus 4 type 1 (Epstein-Barr virus type 1), human herpesvirus 4 type 2 (Epstein-Barr virus type 2), human herpesvirus 5 strain AD169, human herpesvirus 5 strain Merlin Strain, human herpesvirus 6A, human herpesvirus 6B, human herpesvirus 7, human herpesvirus 8 type M, human herpesvirus 8 type P and Human Cyotmegalovirus), human immunodeficiency viruses (HIV) (such as HIV 1 and HIV 2), human metapneumoviruses, human papillomaviruses, human parainfluenza viruses (such as human parainfluenza virus 1-3), human parechoviruses, human parvoviruses (such as human parvovirus 4 and human parvovirus B 19), human respiratory syncytial viruses, human rhinoviruses (such as human rhinovirus A and human rhinovirus B), human spumaretroviruses, human T-lymphotropic viruses (such as human T-lymphotropic virus 1 and human T-lymphotropic virus 2), Human polyoma viruses, Hypoviruses, Leviviruses, Luteoviruses, Lymphocytic choriomeningitis viruses (LCM), Mamaviruses, Namaviruses, Nidovirales, Nodaviruses, Orthomyxoviruses (such as Influenza viruses), Partitiviruses, Paramyxoviruses (such as Measles virus and Mumps virus), Picomaviruses (such as Poliovirus, the common cold virus, and Hepatitis A virus), Potyviruses, Poxviruses (such as Variola and Cowpox), Sequiviruses, Reoviruses (such as Rotavirus), Rhabdoviruses (such as Rabies virus), Rhabdoviruses (such as Vesicular stomatitis virus, Tetraviruses, Togaviruses (such as Rubella virus and Ross River virus), Tombusviruses, Totiviruses, Tymoviruses, and Noroviruses among others.

iii. Bacterial Pathogen

Specific examples of bacterial pathogens include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylo bacterjejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (such as Mycobacterium leprae, Mycobacterium tuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

iv. Fungal Pathogens

Exemplary fungal pathogens include one or more of Trichophyton rubrum, T. mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), and Stachybotrys (such as Stachybotrys chartarum).

### Parasites

Exemplary parasitic organisms include Malaria (Plasmodium falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis) or Coccidiosis (Eimeria species).

Non-limiting examples of suitable viral antigens include: influenza HA, NA, M, NP and NS antigens; HIV p24, pol, gp41 and gpl20; Metapneumovirus (hMNV) F and G proteins; Hepatitis C virus (HCV) E1, E2 and core proteins; Dengue virus (DEN1-4) E1, E2 and core proteins; Human Papilloma Virus L1 protein; Epstein Barr Virus gp220/350 and EBNA-3A peptide; Cytomegalovirus (CMV) gB glycoprotein, gH glycoprotein, pp65, IE1 (exon 4) and ppl50; Varicella Zoster virus (VZV) IE62 peptide and glycoprotein E epitopes; Herpes Simplex Virus Glycoprotein D epitopes, among many others. The antigenic polypeptides can correspond to polypeptides of naturally occurring animal or human viral isolates, or can be engineered to incorporate one or more amino acid substitutions as compared to a natural (pathogenic or non-pathogenic) isolate. Exemplary antigens are listed below:

Exemplary antigens of interest (target antigens)

| | Exemplary Antigen Sequences from the | SEQ ID |
|---|---|---|
| | Antigens of interest | NO: |
| Viral Antigens | | |
| BK | TLYKKMEQDVKVAHQ | 70 |
| | GNLPLMRKAYLRKCK | 71 |
| | TFSRMKYNICMGKCI | 72 |
| JC | SITEVECFL | 73 |
| Epstein-Barr (EBV) | QPRAPIRPI | 74 |
| cytomegalovirus (CMV) | NLVPMVATV | 75 |
| HPV | YMLDLQPET(T) | 76 |
| HPV E7₄₃₋₆₂ | GQAEPDRAHYNIVTFCCKCD | 87 |
| HPV E7₄₉₋₅₇ | CRAHYNIVTF | 88 |
| Influenza A (HA) | GILGFVFTL | 77 |
| Fungal Antigen | | |
| Blastomyces | CELDNSHEDYNWNLWFKWCSGHGR | 78 |
| dermatitidis | TGHGKHFYDCDWDPSHGDYSWYLW | 79 |
| | DPSHGDYSWYLWDYLCGNGHHPYD | 80 |
| | DYLCGNGHHPYDCELDNSHEDYSW | 81 |
| | DPYNCDWDPYHEKYDWDLWNKWCN | 82 |
| | KYDWDLWNKWCNKDPYNCDWDPYH | 83 |

vi. Malignancies Including Tumors

The disclosed methods are of use to treat any tumor. In some embodiments, the disclosed methods are combined with a vaccine or tumor antigen. Examples of hematological tumors include leukemias, including acute leukemias (such as 11 q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

Exemplary tumors and their tumor antigens (antigens produced by tumor cells that can stimulate tumor-specific T-cell immune responses) are shown below.

Exemplary tumors and their tumor antigens

| Tumor | Tumor Associated Target Antigens |
|---|---|
| Acute myelogenous leukemia | Wilms tumor 1 (WT1), PRAME, PR1, |
| | proteinase 3, elastase, cathepsin G |
| Chronic myelogenous leukemia | WT1, PRAME, PR1, proteinase 3, |
| | elastase, cathepsin G |

These exemplary antigens are of use with the disclosed methods, for the treatment of tumors. In one non-limiting example, the tumor is melanoma and the antigen is Trp2.

The disclosed chemical conjugates can also be used in a personal medicine application, wherein a tumor antigen is identified in a particular tumor, in a subject of interest, and that antigen is utilized with the disclosed chemical conjugates.

### Additional Agents

The disclosed chemical conjugates can be administered singularly (i.e., sole therapeutic agent of a regime), or can be administered in combination with another active agent. These can be combined in a single immunomodulatory composition or administered in separate compositions. This disclosure encompasses compositions including one or more of the disclosed chemical conjugates and one or more additional agents, such as chemotherapeutic agents, antiviral agents, and/or antibiotics. In some embodiments, one or more chemical conjugates, as disclosed herein, can be administered in coordination with a regime of one or more other active agents such as antibiotics, antiviral agents, and chemotherapeutic agents. The one or more active agents can be administered sequentially or simultaneously with the one or more chemical conjugates.

Examples of chemotherapeutic agents are alkylating agents, antimetabolites, natural products, or hormones and their antagonists. Examples of alkylating agents include monomethyl auristatin E (MMAE), monomethyl auristatin F (MMAF), nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone). Examples of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech). The chemotherapeutic can be Abraxane.

The disclosed chemical conjugates can be administered with an antibiotic. Antibiotics (antibacterial agents) are compounds that kill or slows down the growth of bacteria. The successful outcome of antimicrobial therapy with antibacterial compounds depends on several factors. These include host defense mechanisms, the location of infection, and the pharmacokinetic and pharmacodynamic properties of the antibiotic. A variety of antibiotics are known, including those that target the bacterial cell wall (for example, penicillins and cephalosporins) or the cell membrane (for example, polymixins), or interfere with essential bacterial enzymes (for example, quinolones and sulfonamides). Antibiotics include, but are not limited to, clindamycin, erythromycin, tetracycline, minocycline, doxycycline, penicillin, ampicillin, carbenicillin, methicillin, cephalosporins, vancomycin, and bacitracin, streptomycin, gentamycin, chloramphenicol, fusidic acid, ciprofloxin and other quinolones, sulfonamides, trimethoprim, dapsone, isoniazid, teicoplanin, avoparcin, synercid, virginiamycin, cefotaxime, ceftriaxone, piperacillin, ticarcillin, cefepime, cefpirome, rifampicin, pyrazinamide, ciprofloxacin, levofloxacin, enrofloxacin, amikacin, netilmycin, imipenem, meropenem, inezolid, pharmaceutically acceptable salts thereof, and prodrugs thereof.

The disclosed chemical conjugates can also be used with a programmed death (PD)-1 antagonist. In some embodiments, the PD-1 antagonist inhibits the binding of PD-1 to programmed death ligand (PD-L)1. In some embodiments, the PD-1 antagonist inhibits the binding of PD-1 to PD-L2. In some embodiments, PD-1 binding antagonist inhibits the binding of PD-1 to both PD-L1 and PD-L2.

In some embodiments, the PD-1 binding antagonist is an antibody, such as a monoclonal antibody. The antibody can specifically bind PD-1, PD-L1 or PD-L2. In some embodiments, PD-L1 binding antagonist inhibits the binding of PD-L1 to PD-1. In some embodiments, the antibody is an antibody fragment selected from the group consisting of Fab, Fab′-SH, Fv, scFv, and (Fab′)₂ fragments.

Exemplary monoclonal antibodies that specifically bind to human PD-1, and are of use in the present methods, are disclosed in U.S. Pat. Nos. 7,521,051, 8,008,449, and 8,354,509. Specific anti-human PD-1 monoclonal antibodies include: MK-3475, a humanized IgG4 monoclonal antibody with the structure described in WHO Drug Information, Vol. 27, No. 2, pages 161-162 (2013), nivolumab (BMS-936558), a human IgG4 monoclonal antibody with the structure described in WHO Drug lnformation, Vol, 27, No, 1, pages 68-69 (2013), and pidilizumab (CT-011, also known as hBAT or hBAT-1); and the humanized antibodies h409A11, h409A16 and h409A17, which are disclosed in PCT Publication No. WO2008/156712. Exemplary monoclonal antibodies that specifically bind to human PD-L1, are disclosed in PCT Publication No. WO2013/019906, WO2010/077634 A1 and U.S. Pat. No. 8,383,796. Monoclonal antibodies that that specifically bind PD-L1 include MPDL3280A, BMS-936559, MEDI4736, MSB0010718C and an antibodies disclosed in PCT Publication NO. WO2013/019906. In some specific, non-limiting examples, the PD-1 binding antagonist is nivolumab. In other specific non-limiting examples, the PD-1 antagonist is pembrolizumab. In further non-limiting examples, the PD-1 antagonist is CT-011 or AMP-224. The PD-1 antagonist can also be an antisense molecule specific for PD-1, PD-L1 or PD-L2.

An immunoadhesin that specifically binds to human PD-1 or human PD-L1 can also be utilized. An immunoadhesin is a fusion a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are disclosed in PCT Publication Nos. WO2010/027827 and WO2011/066342, both incorporated by reference. These immunoadhesion molecules include AMP-224 (also known as B7-DCIg), which is a PD-L2-FC fusion protein.

The disclosed chemical conjugates can be used with inhibitors of other immune checkpoints such as cytotoxic T-lymphocyte-associated protein 4 (CTLA-4), indoleamine 2,3-dioxygenase (IDO), and T-cell immunoglobulin domain and mucin domain-3 (TIM-3), either alone or in combination, can also be further combined with the disclosed chemical conjugate.

In some embodiments, a CTLA-4 antagonist is used in the methods disclosed herein. The CTLA-4 antagonist can be an antibody that specifically binds CTLA-4. Antibodies that specifically bind CTLA-4 are disclosed in PCT Publication No. WO 2001/014424, PCT Publication No. WO 2004/035607, U.S. Publication No. 2005/0201994, European Patent No. EP1141028, and European Patent No. EP 1212422 B1. Additional CTLA-4 antibodies are disclosed in U.S. Pat. Nos. 5,811,097, 5,855,887, 6,051,227, 6,984,720, 6,682,736, 6,207,156, 5,977,318, 6,682,736, 7,109,003, 7,132,281, 7,452,535, 7,605,238PCT Publication No. WO 01/14424, PCT Publication No. WO 00/37504, PCT Publication No. WO 98/42752, U.S. Published Patent Application No. 2000/037504, U.S. Published Application No. 2002/0039581, and U.S. Published Application No. 2002/086014. Antibodies that specifically bind CTLA-4 are also disclosed in Hurwitz et al., Proc. Natl. Acad. Sci. USA, 95(17):10067-10071 (1998); Camacho et al., J. Clin. Oncol., 22(145): Abstract No. 2505 (2004) (antibody CP-675206); Mokyr et al., Cancer Res., 58:5301-5304 (1998). In some embodiments the CTLA-1 antagonist is Ipilmumab (also known as MDX-010 and MDX-101 and YERVOY®), see PCT Publication No. WO 2001/014424, incorporated herein by reference.

In further embodiments, a B- and T-lymphocyte attenuator (BTLA) antagonist is utilized in the methods disclosed herein. Antibodies that specifically bind BTLA are disclosed, for example, in U.S. Published Patent Application No. 2016/0222114, U.S. Published Patent Application No. 2015/0147344, and U.S. Publisehd Patent Application No. 2012/0288500, all incorporated herein by reference. Biological agents that modulate BTLA activity, specifically using Herpesvirus entry mediator (HVEM) cis complexes are disclosed in U.S. Published Patent Application No. 2014/0220051 and U.S. Published Patent Application No. 2010/0104559, both incorporated herein by reference.

This conjugate can also be used in combination with other immunomodulatory agents, such as other TLR agonists. Examples include imidazoquinoline and derivatives.

The disclosed chemical conjugates can also be used with an antiviral agent such as acyclovir, brivudine, docosanol, famciclovir, idoxuridne, penciclovir, trifluridine, valacyclovir, amantadine, rimantadine, oseltamivir, or zanamivir.

### EXAMPLES

Subunit vaccines are capable of eliciting potent and disease-specific immune responses. It is thus critical to deliver subunit vaccines, such as CpG oligonucleotide adjuvants and peptide antigens, into secondary lymphoid organs, where the majority of vaccine-elicited immune responses are orchestrated. While nanoparticles and microemulsion have been extensively studied for vaccine delivery, molecular vaccines are attractive alternatives in view of their well-defined chemical and pharmacological properties. However, molecular vaccines often have short retention time in lymph nodes before being rapidly drained into the blood circulation, leading to marginal therapeutic potency and, more seriously, systemic toxicity.

Albumin-binding dye Evans Blue was modified to be maleimide-functionalized Evans Blue (MEB), and was conjugated with vaccines as novel albumin-binding molecular vaccines (termed as AlbiVax). Through avidly binding of MEB to endogenous albumin, AlbiVax was efficiently delivered to and retained in lymphoid organs. By radiolabeling MEB with ⁶⁴Cu, a panel of AlbiVax candidates was screened for highest retention in lymph nodes (LNs), and the pharmacological behaviors of the select AlbiVax were quantitatively studied in small animals using positron emission tomography (PET) and radioactivity measurement of ex vivo organs. The total AlbiVax accumulated in inguinal LNs, axillary LNs, and lumbar LNs were up to 7-fold within 3 days, of unconjugated vaccines or vaccines delivered by depot-forming Incomplete Freund Adjuvant (IFA), the current benchmark in clinical trials of cancer therapeutic vaccines.

AlbiVax co-delivered Adjuvant/Ags into the endolysosome of antigen-presenting cells and elicited 21-fold more frequency of Ag-specific CD8⁺ cytotoxic T lymphocytes (CTLs) than clinic benchmark IFA-emulsified CpG, and induced immune memory for >5 months. By leveraging the intrinsic fluorogenicity of MEB, the intracellular behaviors of AlbiVax in antigen presenting cells (APCs) and the intranodal behaviors of LNs were tracked using super-resolution fluorescence confocal microscopy and flow cytometry, which revealed that AlbiVax was efficiently taken up into the endolysosome of APCs both in vitro and in lymph node-residing APCs. AlbiVax induced potent APC immunostimulation, specific T cell priming, and elevated antibody production. Remarkably, MEB-CpG dramatically reduced the systemic dissemination of CpG, thereby significantly reducing the toxicity resulting from systemic inflammation.

MEB was conjugated with tumor-specific antigen to develop tumor-specific AlbiVax. AlbiVax dramatically inhibited progression of established primary or lung metastatic tumors. AlbiVax can be used alone and in combination with other therapeutic modalities, including, but not limited to, PD-1 antibody-based immunotherapy and chemotherapy. When combined with other therapeutics, including immune checkpoint inhibitors, photodynamic therapy, and chemotherapy, the therapeutic efficacy was further improved, which demonstrated versatile synergistic combination therapy of AlbiVax with other therapeutic modalities. Thus, AlbiVax represents a novel class of chemically-defined and pharmacologically-favored immunotherapeutics with both potent therapeutic efficacy and high safety.

### Example 1

### Conjugation and Characterization of MEB-CpG

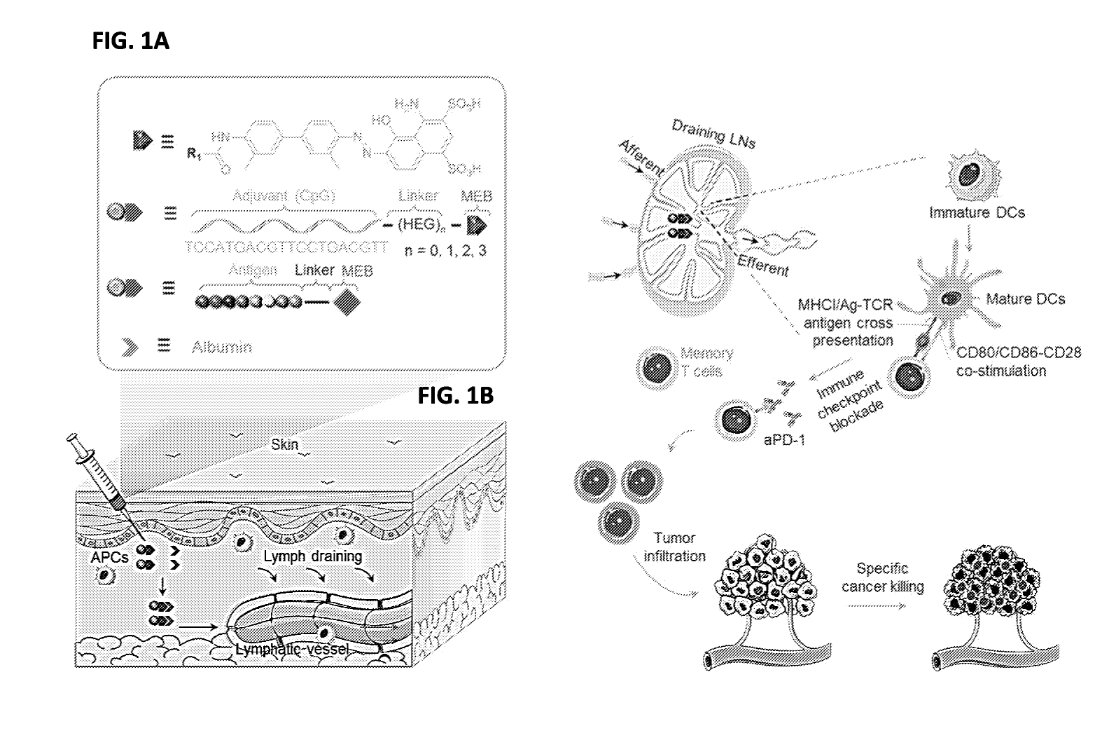

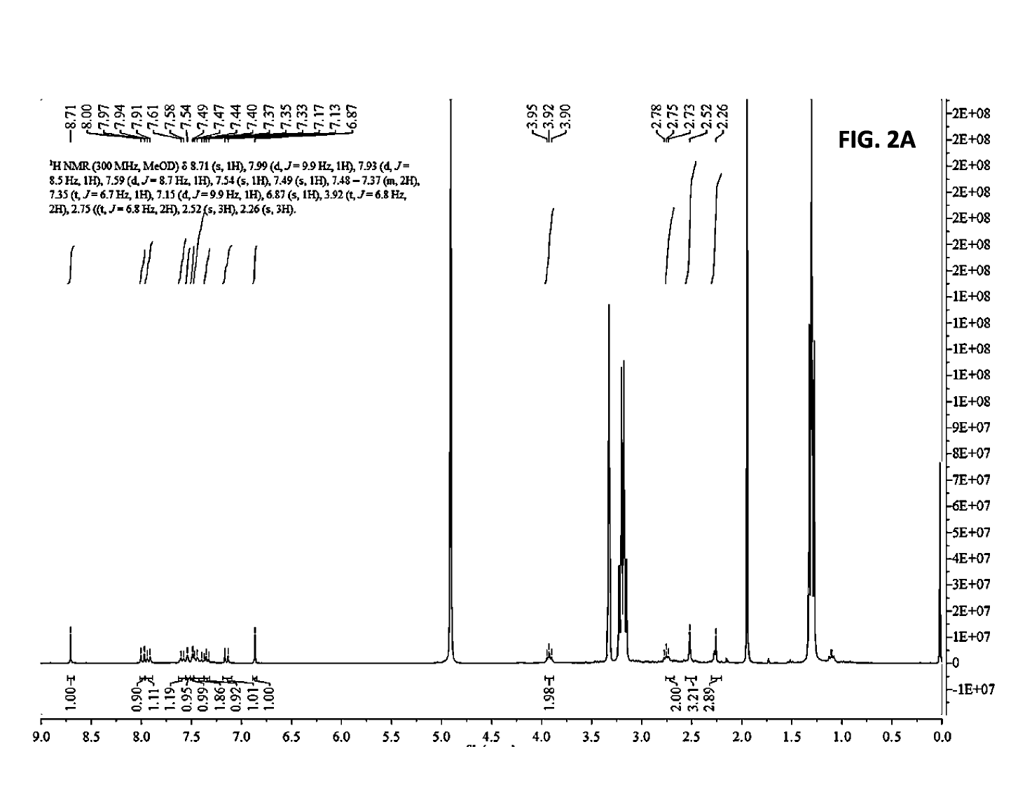

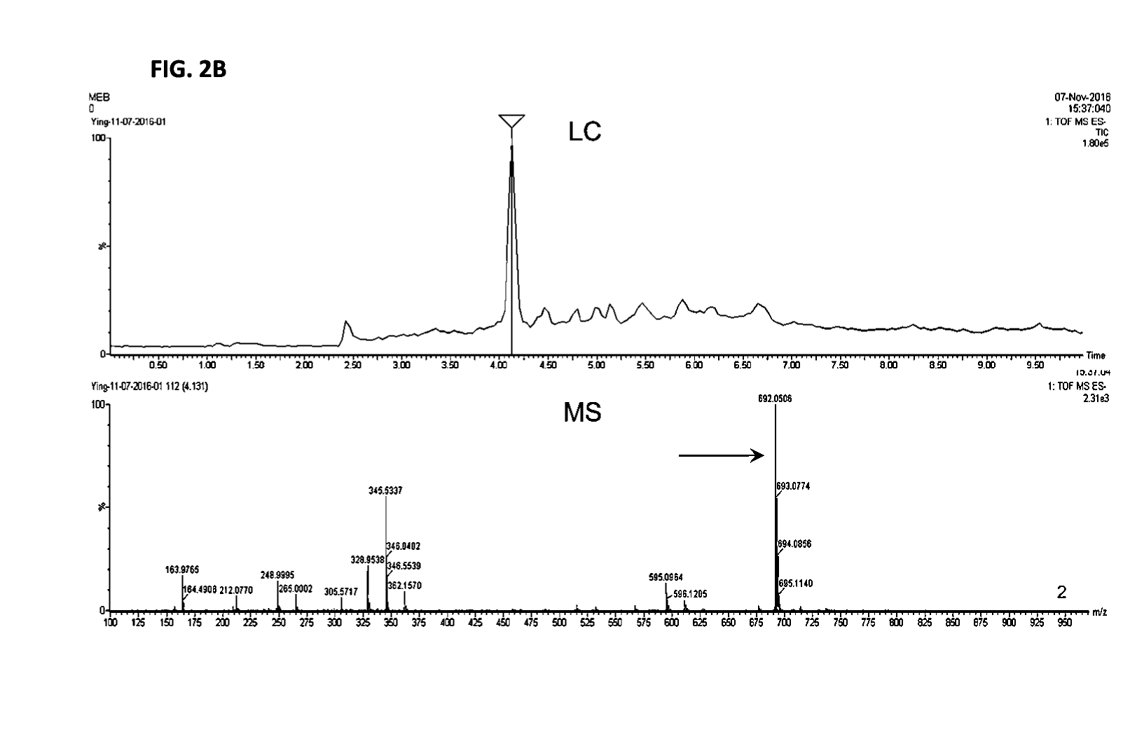

AlbiVax was synthesized by site-specific conjugation of subunit vaccines and functionalized EB derivatives (FIG. 1, 2). A model adjuvant, CpG, was first studied, using thiol-modified CpG and maleimide-functionalized truncated EB (MEB) to prepare MEB-CpG conjugates (denoted as AlbiCpG.). Hexaethyloxy-glycol (HEG) linkers were used to tune the distance between CpG and MEB.

Given that thiol groups are ubiquitous in natural protein antigens and are also easy to be modified onto nucleic acid adjuvants and synthetic peptide antigens, a maleimide group was functionalized on MEB to be conjugated with thiol-modified vaccines via thiol-maleimide conjugation (see FIG. 1). MEB-CpG was first studied as a model using CpG 1826, which was functionalized with thiol (—SH) on the 3′ end.

CpG 1826

(SEQ ID NO: 2)

TCCATGACGTTCCTGACGTT
GpC (control)

(SEQ ID NO: 34)

TCCATGAGCTTCCTGAGCTT

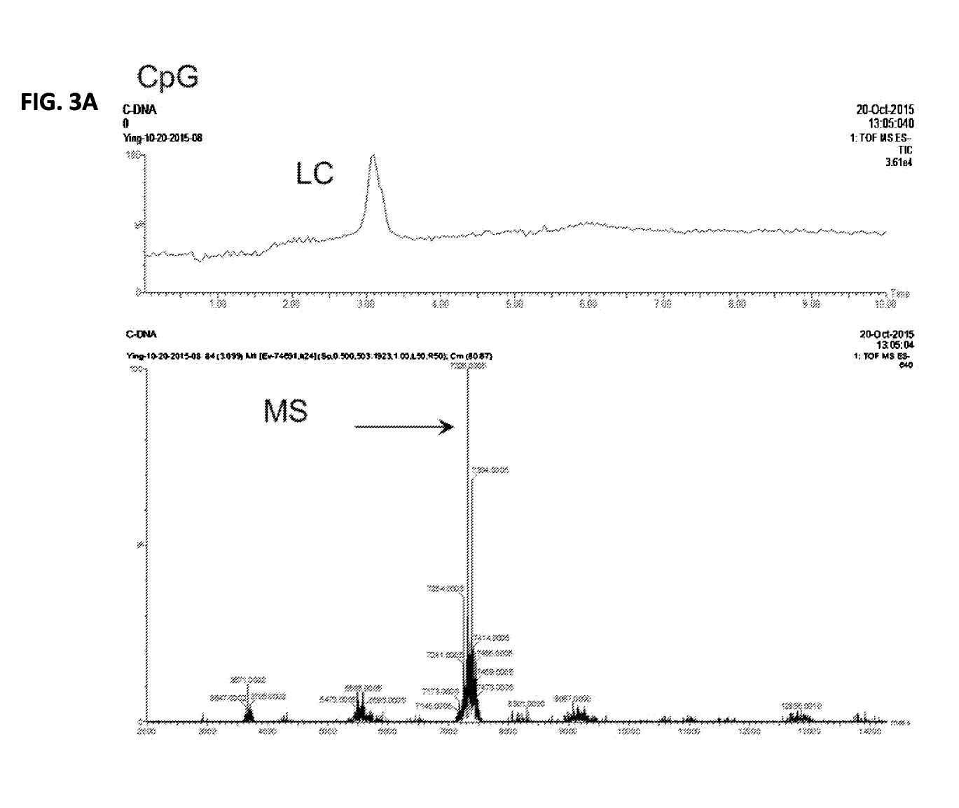

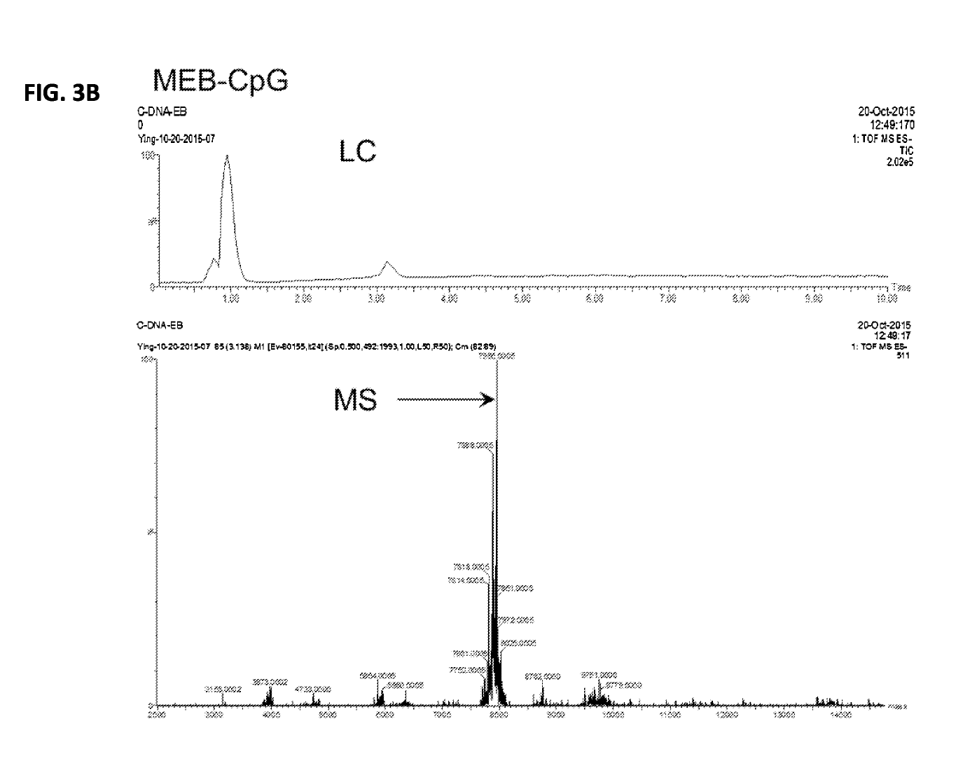

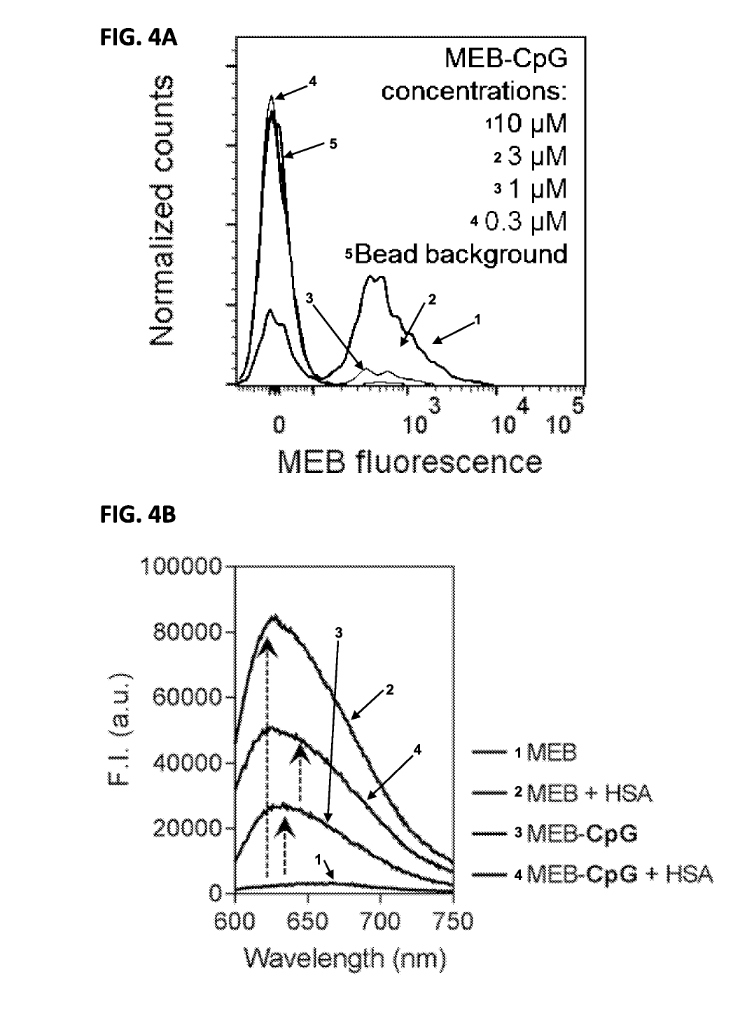

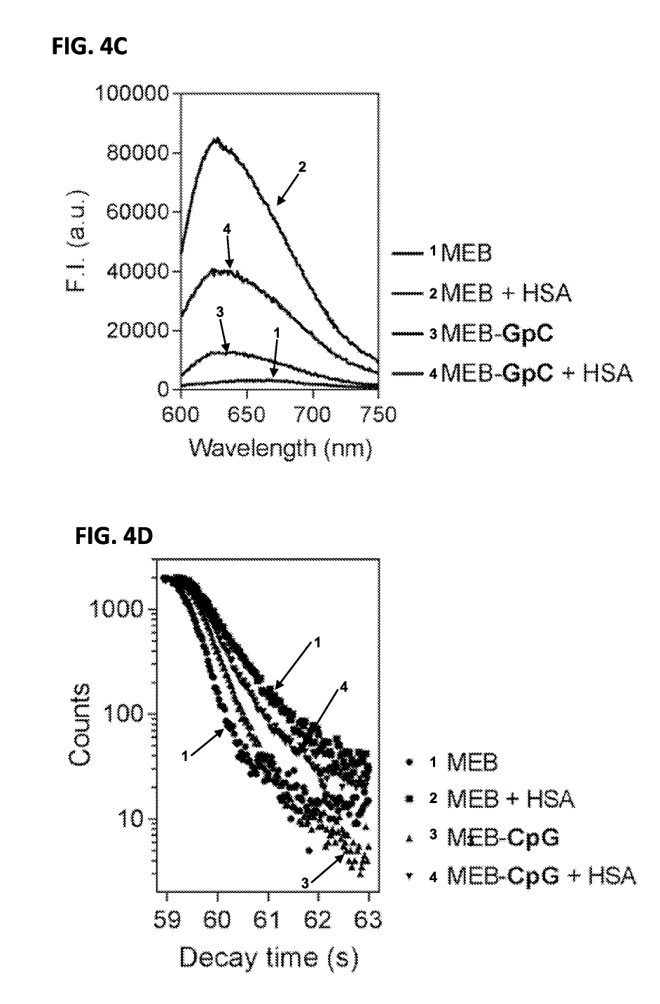

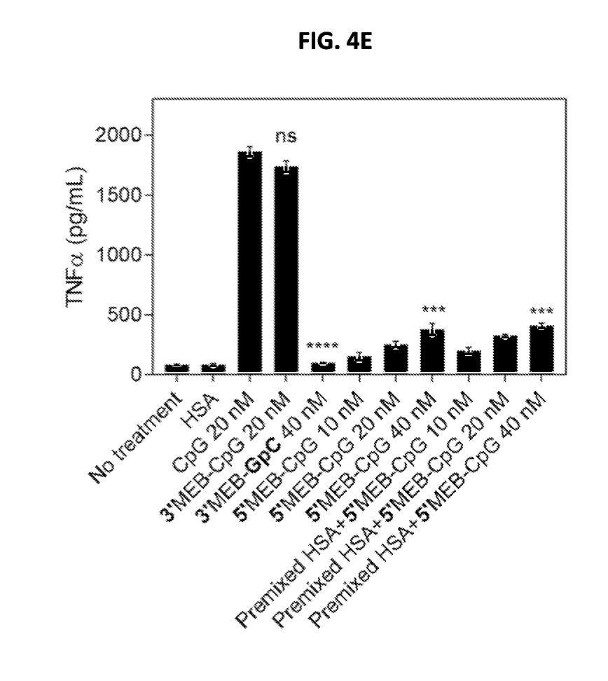

After a thiol-maleimide conjugation at room temperature for half an hour, the product was purified simply using size-exclusion column. Control GpC derived from mutating the CG islands in CpG into GC was used to synthesize MEB-GpC. The molecular weights of MEB-CpG and MEB-GpC were verified using liquid chromatography-electrospray ionization-tandem mass spectrometry LC-MS (FIG. 3). Similarly, two another conjugates, denoted as MEB-HEG-CpG and MEB-HEG2-CpG, were synthesized with 1 and 2 hexaethyloxy-glycol units as the linkers between MEB and CpG. The intrinsic optical characteristics of MEB allowed us to monitor CpG-MEB conjugation and the albumin-binding ability of MEB-CpG. MEB-CpG preserved the unique absorbance of MEB; interestingly, conjugation of MEB onto CpG dramatically activated the fluorescence of MEB to about one third of the fluorescence intensity fully activated by albumin (FIGS. 4A-4D). The spontaneous fluorescence activation upon DNA conjugation is presumably due to the enhanced conformational rigidity resulting from noncovalent associations such as π-π interaction between nucleobases and MEB.

The intrinsic fluorogenicity of MEB-CpG allowed its behaviors to be tracked in cells and in animals. The ability of MEB-CpG to bind to albumin was confirmed by the enhancement of the fluorescence intensity (FIG. 4B) and the fluorescence lifetime (FIG. 4D) of MEB-CpG in the presence of human serum albumin (HSA). Compared to free MEB-HSA complexes, the less enhancement of fluorescence intensity and fluorescence lifetime of MEB-CpG-HSA complexes suggest that the albumin binding ability of MEB is likely affected in MEB-CpG. Similarly, the fluorescence of MEB was also activated in MEB-GpC (FIG. 4C), and both the fluorescence intensity and the fluorescence lifetime were enhanced upon binding to albumin (FIG. 4). Even though the activation of MEB fluorescence upon DNA conjugation provides insight about the interaction between DNA and MEB moiety, it was possible that this interaction could interfere with the binding ability of AlbiVax with albumin. An inert linker between CpG and the MEB moiety addressed this concern. Three AlbiVax candidates, namely MEB-GpC, MEB-HEG-CpG, and MEB-HEG2-CpG, which have a linker of approximately 1 nm, 3 nm, and 5 nm, respectively, between CpG and MEB were synthesized.

### Example 2

### Preparation Exemplary Chemical Conjugate

To a 100 ml round bottom flask containing o-tolidine (4.3 g) and methylene chloride (40 ml) was added di-t-butyldicarbonate (4.4 g). The mixture was stirred at room temperature overnight. The reaction was concentrated and the residue was purified by chromatography on silica gel to give 3.2 g of N-Boc-2-tolidine (1). N-Boc-2-tolidine (0.46 g, 1.47 mmol) was dissolved in acetonitrile (10 ml) in a glass vial and cooled to 0° C., then hydrochloric acid (0.3 M, 15 ml) was added. Cold sodium nitrite solution (0.31 g in 5 ml water) was added dropwise and stirred for 20 min, and the solution turned bright yellow. This solution was added dropwise to another glass vial containing 1-amino-8-naphthol-2,4-disulfonic acid monosodium salt (0.59 g) and sodium bicarbonate (0.49 g) in water (20 ml) at 0° C. The reaction was deemed complete by LC/MS and the reaction was lyophilized without further purification to provide the Boc-tEB (truncated EB) (2) product. The Boc-tEB product was added to a solution of 80% TFA, 10% 1,2-ethanedithiol and 10% thioanisole and stirred until the reaction was complete. The mixture was diluted with water (100 ml) and loaded onto a C18 chromatography cartridge (3×15 cm). The column was washed with water and then with 80% ethanol to elute the desired product. After evaporation of the solvent in the eluent, 0.6 g of 80% pure product of tEB (3) was obtained. A small amount of product 3 was further purified by HPLC. To a solution of purified tEB (30 mg) in methanol (4 ml), DIPEA (50 μl) and maleic anhydride (160 mg) were added, and the reaction was stirred at room temperature for 2 h. When the reaction to form intermediate was complete as judged by HPLC, the solvent was evaporated, acetic anhydride (1 ml) was added and the reaction was heated at 105° C. for 30 min. When LC/MS showed complete conversion to desired product, the mixture was diluted with water (16 ml) and purified on a Waters Xterra C18 chromatography column running a linear gradient from 5% A (0.1% TFA in acetonitrile) and 95% B (0.1% TFA in water) for 2 min and increasing A to 65% in 30 min. The desired product was collected and lyophilized to obtain MEB.

DNA was synthesized on a 1 μmole scale of solid phase synthesis on an ABI 392 DNA synthesizer (Applied Biosystems). Phosphoramidites and other materials used for DNA synthesis were purchased from Glen Research (Sterling, Va.) or Chemgenes (Wilmington, Mass.). DNA was deprotected at 65° C. in solution of methylamine and ammonium oxide (1:1) for 30 min. DNA was purified by using C18 column in reverse phase HPLC (Dionex Ultimate 3000, ThermoFisher Scientific, Waltham, Mass.), with 0.1 M triethylammonium acetate (TEAA) and acetonitrile as mobile phase and stationary phase, respectively. Dimethoxytrityl (DMT) protecting group was removed from DNA by treating with 0.5 M acetic acid. DNA was desalted, and quantified on a Genesys 10S UV-Vis spectrometer (ThermoFisher Scientific, Waltham, Mass.). All DNAs were modified to have phosphothiolate backbone for nuclease resistance. Amine, alkyne or azide, thiol, or HEG were modified according to manufacturer's instructions.

MEB-modified CpG or derivatives were synthesized using MEB and terminal thiol (at 3′-end or 5′-end)-modified DNA. Thiol-modified DNA was pretreated with DTT (0.1 M) in PBS for 1 h at 37° C. to cleave dithiol bond, followed by desalting using a NAPS column in sodium ascorbate buffer (0.1%) to remove DTT and the thiol-appending small fragment cleaved from DNA. The resulting DNA (20 nmole) was mixed with MEB (100 nmole) in 500 uL sodium ascorbate buffer (0.1%) in PBS and left for 30 min at room temperature. The resulting product was purified again using a NAPS column to remove excess MEB.

MEB-modified Trp2 peptide was synthesized using MEB and Trp2 modified with N-terminal cysteine. Trp2 was dissolved in DMF, followed by adding MEB dissolved in DMF drop-by-drop. The reaction mixture was agitated at room temperature for at least 1 day, until unreacted Trp2 was not detectable by LC-MS. MEB-Trp2 was purified using a C18 column on HPLC.

### Example 3

### Efficient and Sustainable LN-Targeted Delivery and Prolonged Retention of MEB-CpG in LNs

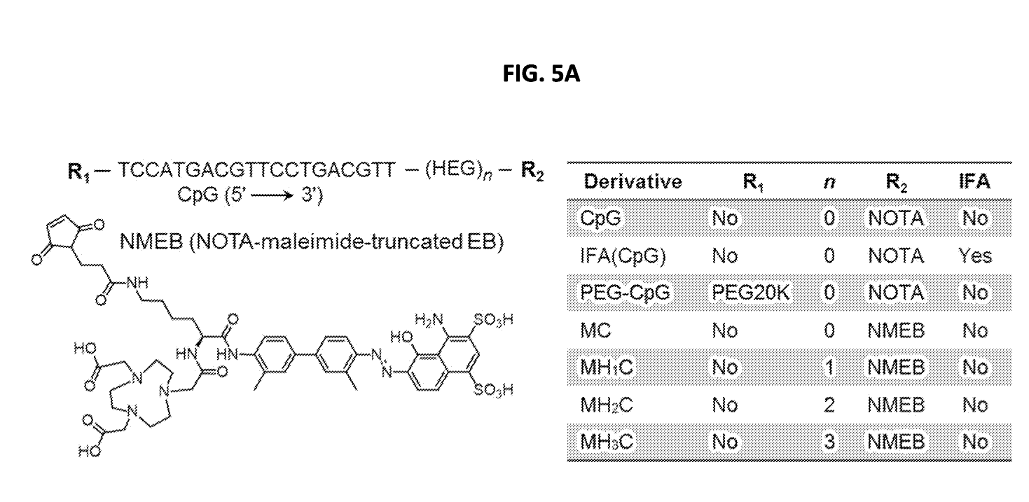

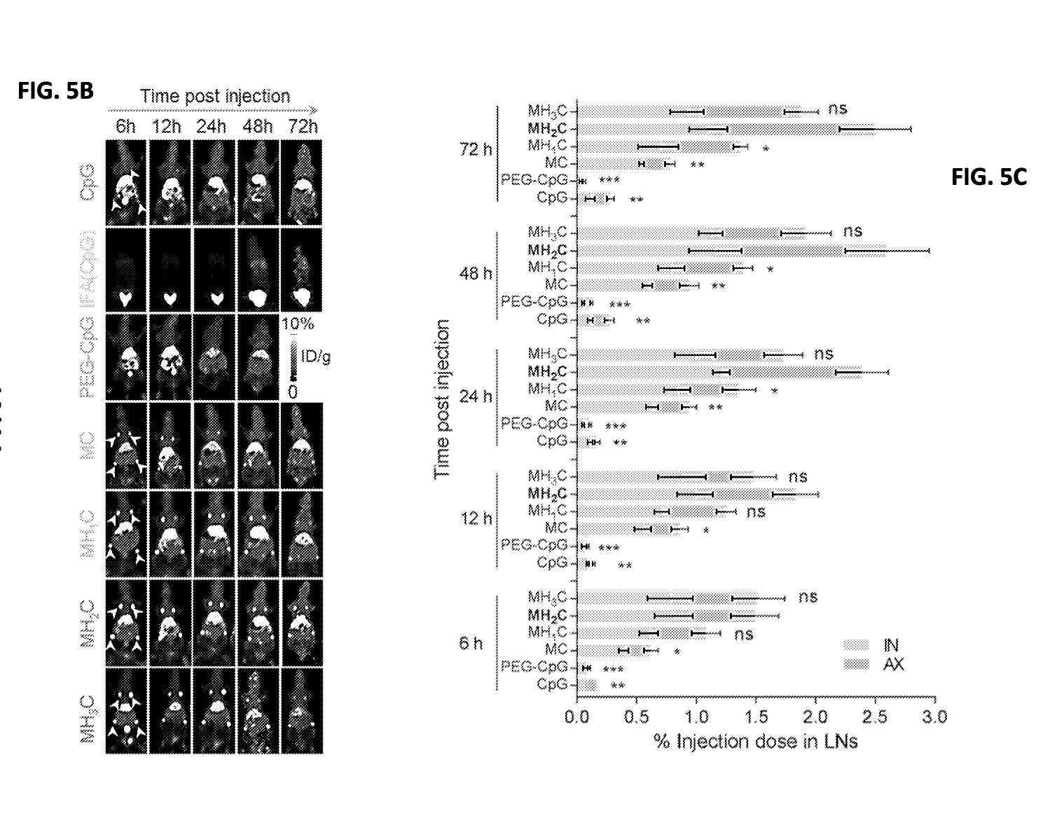

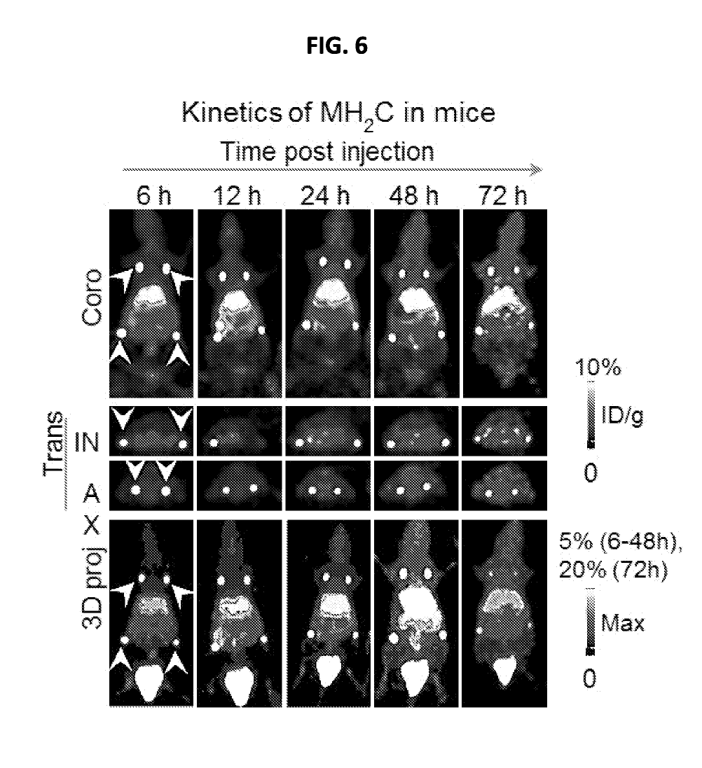

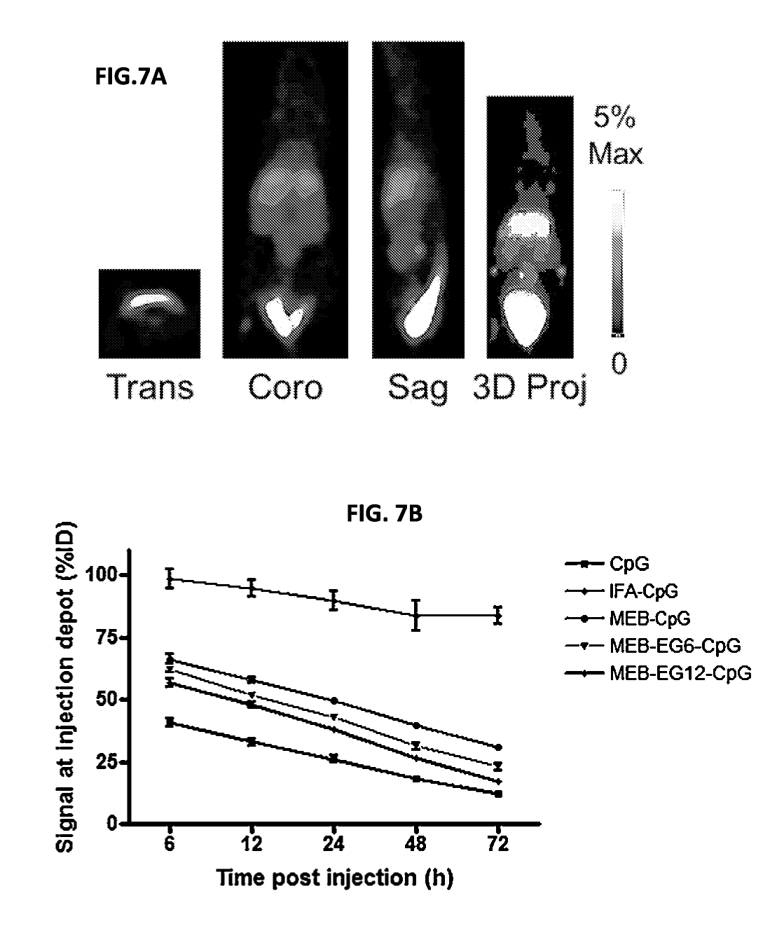

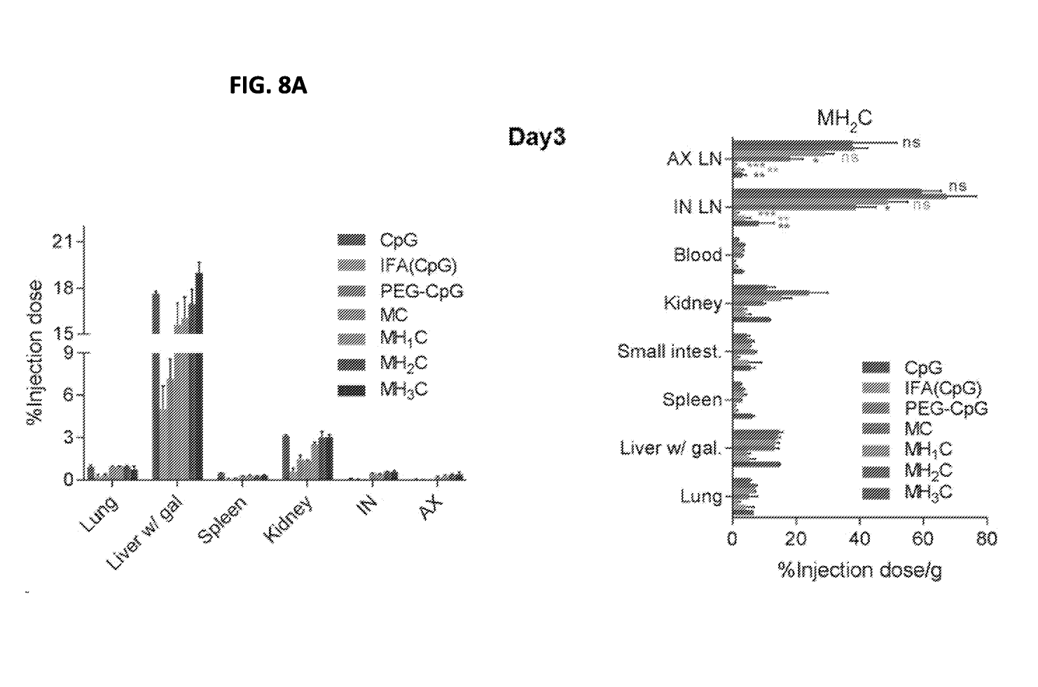

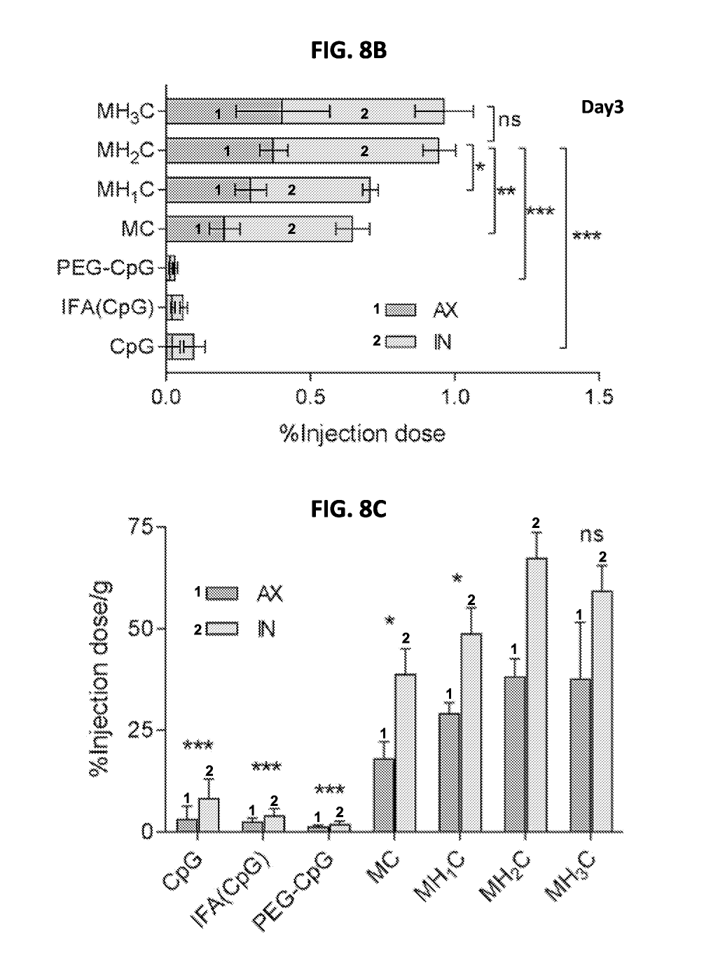

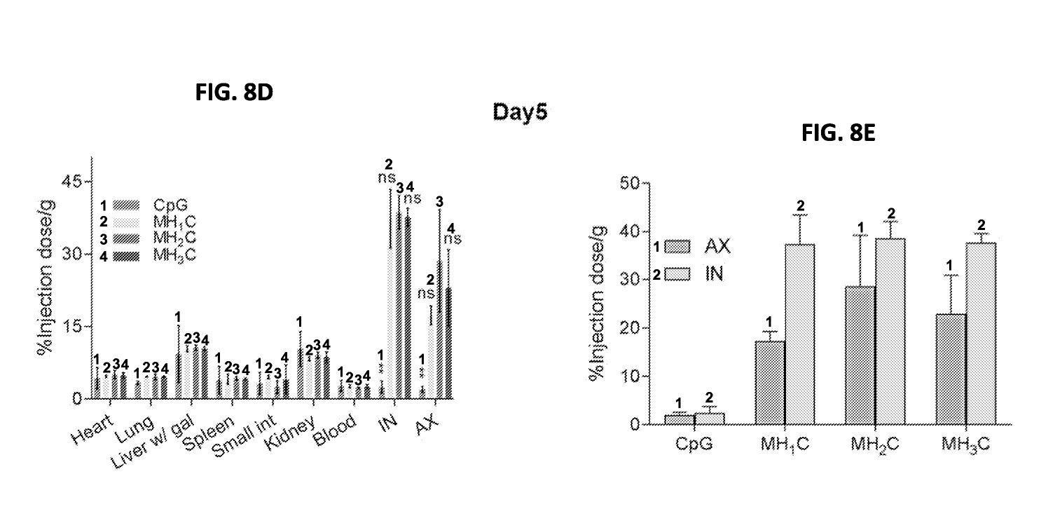

A group of AlbiCpG candidates with linkers of 0, 1, 2, and 3 units of HEGs (˜1, 3, 5, and 7 nm respectively) were quantitatively screened in BALB/c mice for optimal LN-targeted delivery by positron emission tomography (PET) and ex vivo γ counting using ⁶⁴Cu (t_{1/2}: 12.7 h). In contrast to semiquantitative optical imaging, PET is able to quantitatively and noninvasively determine radiolabeled compounds in a whole body in both preclinical studies and clinical applications. ⁶⁴Cu, which has a half-life of 12.7 h, was chosen for PET imaging. To label ⁶⁴Cu onto AlbiCpG, a 1,4,7-triazacyclononane-triacetic acid (NOTA)-MEB (NMEB) was synthesized and conjugated with CpG derivatives (FIG. 5A). Mice were s.c. injected with ⁶⁴Cu-radiolabeled AlbiCpG, followed by PET imaging to reveal its 3D biodistribution in mice over 3 days (FIG. 5B). By ⁶⁴Cu-labelling using NOTA, we also studied free CpG injected in PBS or within emulsion IFA, which is likely the most widely used adjuvant in clinic trials, and CpG conjugated with a terminal polyethylene glycol (PEG, MW: ˜20 K). Based on PET, decay-corrected radioactivity in draining inguinal (IN) LNs, and axillary (AX) LNs. LNs were quantified as the percentage of injection dose (% ID) (FIG. 5C). As shown in FIG. 5B, all four AlbiCpG candidates was retained in LNs significantly more than free CpG, IFA(CpG), and PEG-CpG. Among four AlbiCpG candidates, MEB-(HEG)₂-CpG (FIG. 5B-5C, FIG. 6) had the highest LN retention at 6 h post injection [total % ID in IN, AX, and LU LNs: 0.72, 1.55, 1.81, and 1.74 for MEB-CpG, MEB-HEG-CpG, MEB-(HEG)₂-CpG, and MEB-(HEG)₃-CpG, respectively]. The area under curve (AUC) of MEB-CpG, MEB-HEG-CpG, MEB-(HEG)₂-CpG, and MEB-(HEG)₃-CpG were 3, 5.5, 6.5, and 6.1-folds greater than that of CpG within 3 days. IFA(CpG) was tremendously trapped in the injection depot but little was accumulated in LNs (FIG. 7). Consistently, both draining IN and AX LNs had significantly higher radioactivity density than any other organs, as found by γ counting of the radioactivity of excised organs (FIG. 8). For instance, on day3, the % ID/g (injection dose per gram of tissue) in IN LNs were 38.8±6.3, 48.9±6.2, 67.4±9.3, and 59.5±6.02 for MEB-CpG, MEB-HEG-CpG, MEB-(HEG)₂-CpG, and MEB-(HEG)₃-CpG, respectively. In contrast, CpG delivered via IFA showed marginal increased accumulation in LNs relative to free CpG. Based on these screening, MEB-(HEG)₂-CpG was selected for further study.

CpG showed no apparent accumulation in any of the LNs but was found in many other organs caused by systemic dissemination. CpG delivered by IFA resulted in undetectable levels of accumulation in any of these organs at early time points, yet much activity was observed in organs other than LNs at later time points. It should also be noted that a large amount of injected vaccines were retained in the injection depot. The retained amount in injection depot gradually decreased due to sustainable draining to LNs (FIG. 7).

Free CpG showed only 8.3±4.7% ID/g in inguinal LNs and 3.2±1.2%/g in axillary LNs, and CpG delivered via IFA showed only 4.0±1.7% ID/g in inguinal LNs and 2.6±0.8% ID/g in axillary LNs.

Aside from LNs, relatively high radioactivity density was observed in some other organs such as liver, kidney, and small intestine, which was presumably because of the metabolism and renal clearance of nucleic acids, and the high amount of albumin in liver (FIGS. 8A-8C). The blood had less than 4% ID/g for all tested formulations. On day 5 post injection, even though the radioactivity of ⁶⁴Cu-labeled vaccines was too low to be sensitively detected by PET, the radioactivity of ex vivo organs were sensitively measured to determine the biodistribution profile (FIGS. 8D-8E).

### Example 4

### LN-Targeted Delivery of Vaccine Candidates

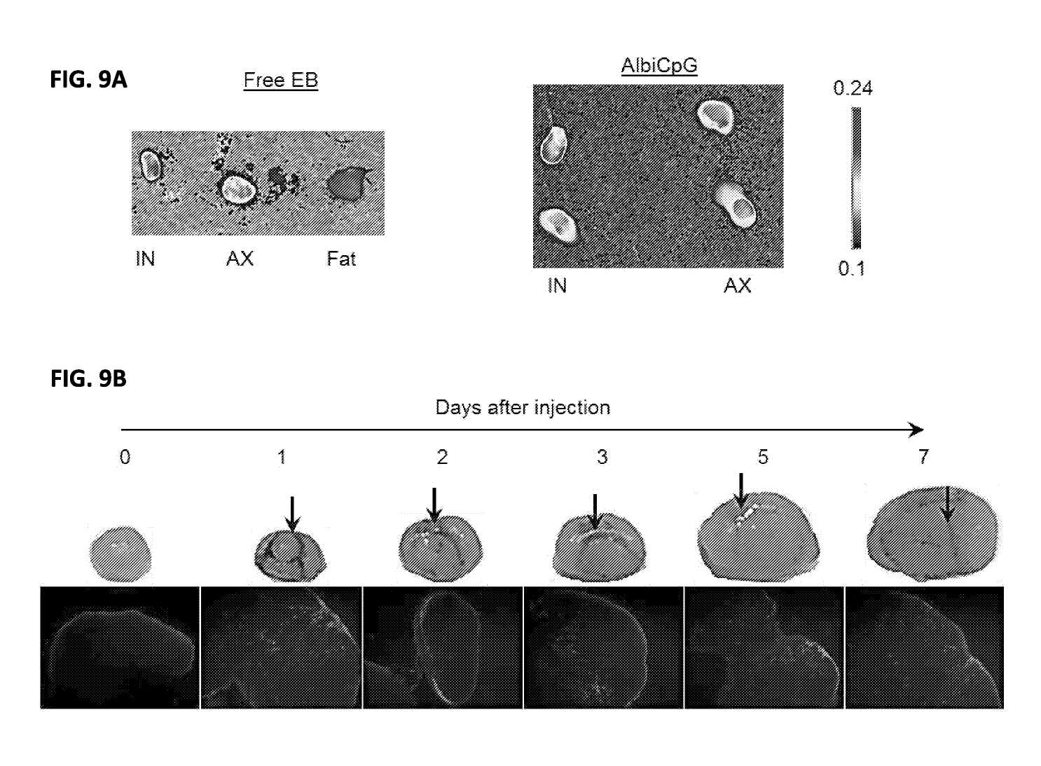

To look closer at the in vivo behaviors of AlbiVax, AlbiVax was subcutaneously injected at the base of tail, followed by excision of inguinal LNs and axillary LNs at specified time points post injection. Taking advantage of the intrinsic fluorescence of MEB, the accumulation efficiency of AlbiVax in excised LNs was semiquantitatively evaluated by optical imaging. As a result, strong MEB fluorescence (FIG. 9A) was observed in the LNs of mice, which is consistent with the PET results. For intranodal and intracellular localization of AlbiVax, CY5®, a fluorophore with stronger fluorescence intensity than MEB under optimal conditions, was modified on the 5′ end of CpG of AlbiVax. Similarly, strong fluorescence from CY5®-labeled AlbiVax was observed in excised inguinal LNs and axillary LNs, aside from the purplish color of MEB (FIG. 9B).

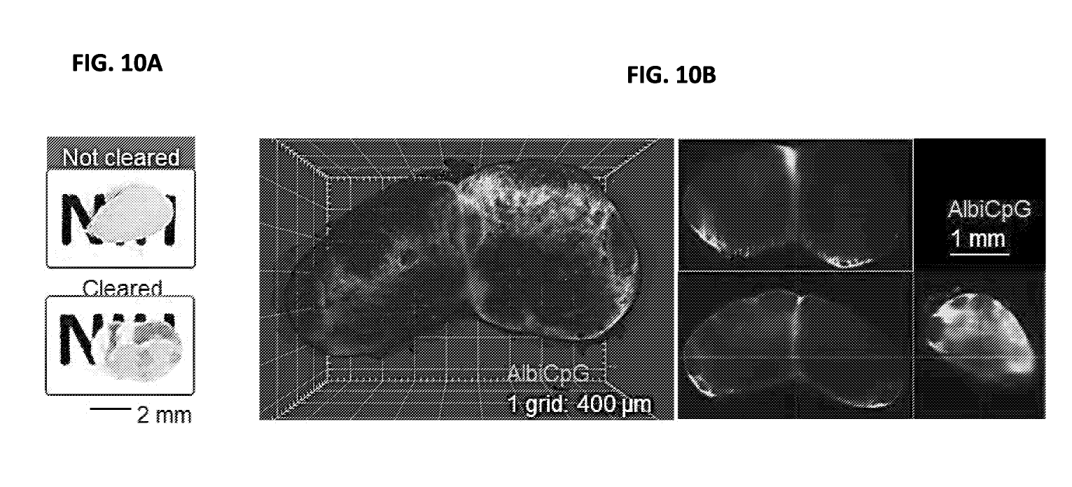

Given efficient LN delivery of AlbiCpG, the intranodal distribution of AlbiCpG was further mapped by light sheet fluorescence microscopy (LSFM), which allows for 3D imaging of whole intact tissues with low photobleaching and toxicity. Specifically, at 1 day post s.c. injection of AlbiCpG labeled with Alexa488 (higher photostability and fluorescence intensity than MEB) in C57BL/6 mice, draining IN LNs were resected and “cleared” to be transparent using a passive CLARITY technique (PACT) (FIG. 10A). LSFM imaging of the cleared LNs mapped the 3D distribution of intranodal AlbiCpG, which was especially abundant in the subcapsular sinus areas and around B cell follicles (FIG. 10B).

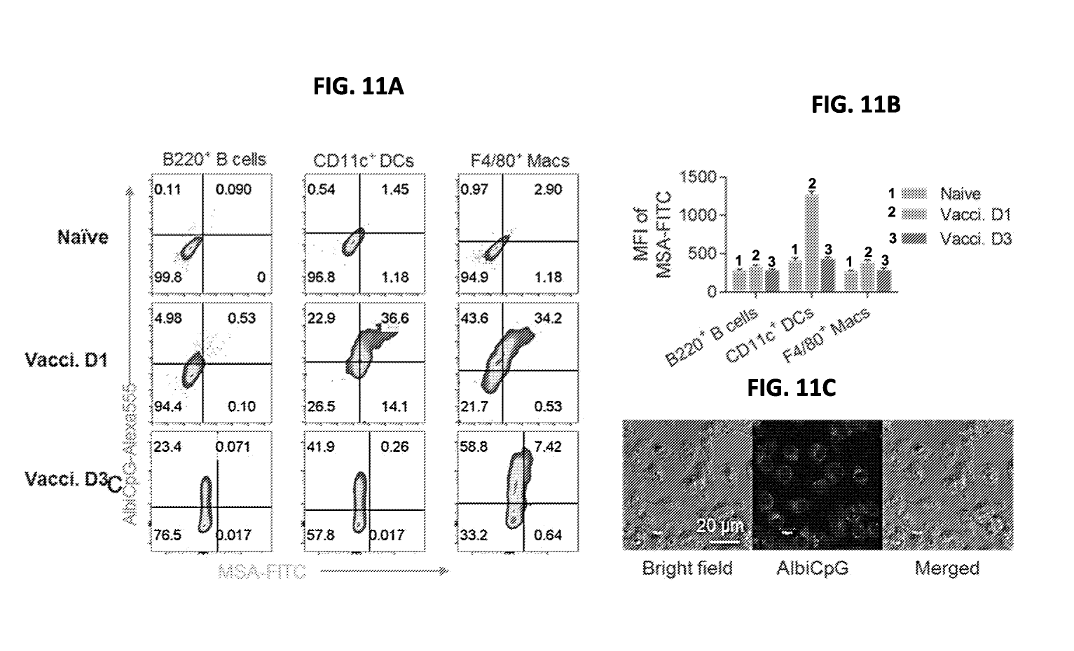

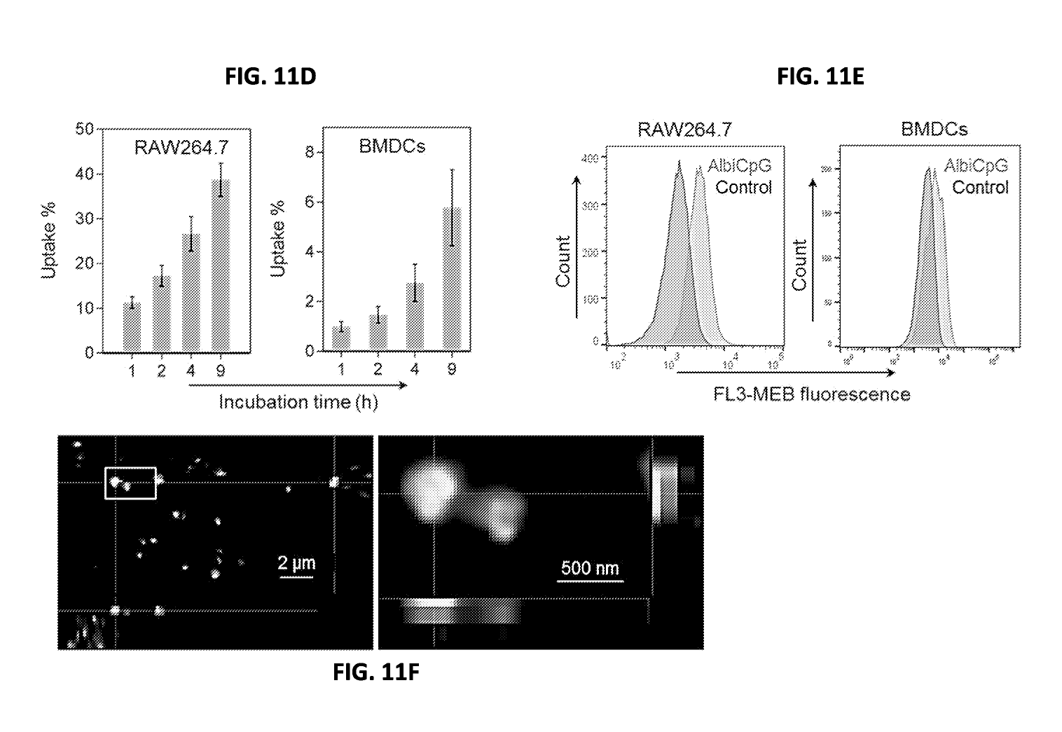

Given abundant intranodal AlbiCpG, the distribution of AlbiCpG in LN-residing lymphocytes, particularly in B220⁺ B cells, CD11c⁺ DC cells, and F4/80⁺ macrophages was further disected. FITC-labeled MSA and Alexa555-labeled AlbiCpG were co-injected s.c. at the tail base of C57BL/6 mice. LN-residing APCs were analyzed by flow cytometry on day1 and day3 post injection (FIG. 11A-11B). On day1, 43% DCs and 27% macrophages were AlbiCpG⁺, among which 28% DCs and 18% macrophages were AlbiCpG+MSA⁺, suggesting uptake of albumin/AlbiCpG complexes; in contrast, only <1% B cells were AlbiCpG⁺ despite 7% of MSA⁺ B cells. On day3, 27% DCs, 55% macrophages, and 14% B cells were AlbiCpG+; MSA-FITC was likely exhausted and was marginally detected. We further examined the intracellular delivery of AlbiCpG to APCs, a pivotal process for efficient antigen cross presentation and immunomodulation. Efficient in vitro uptake of AlbiCpG by RAW264.7 macrophages and BMDCs was shown by confocal microscopy, γ counting of ⁶⁴Cu-labeled AlbiCpG, and flow cytometry (FIG. 11c-e). Super-resolution confocal microscopy discovered ubiquitous AlbiCpG-Alexa555 in BMDC endolysosome, and intriguingly, primarily on the membrane of endolysosome likely because AlbiCpG bound with TLR9 on membrane (FIG. 1f).

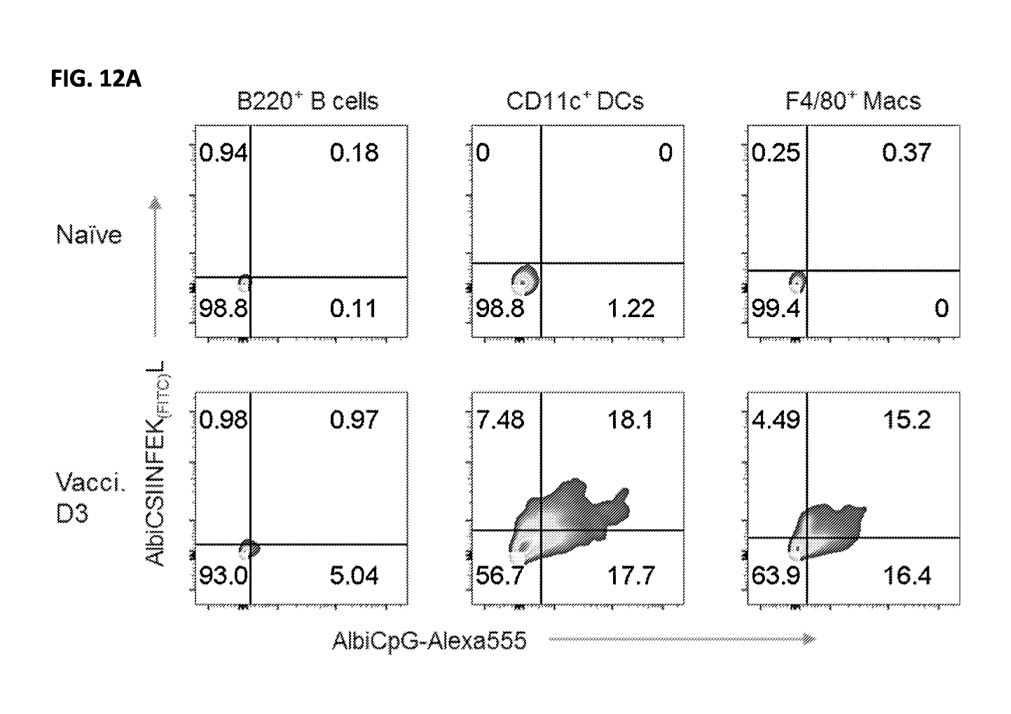

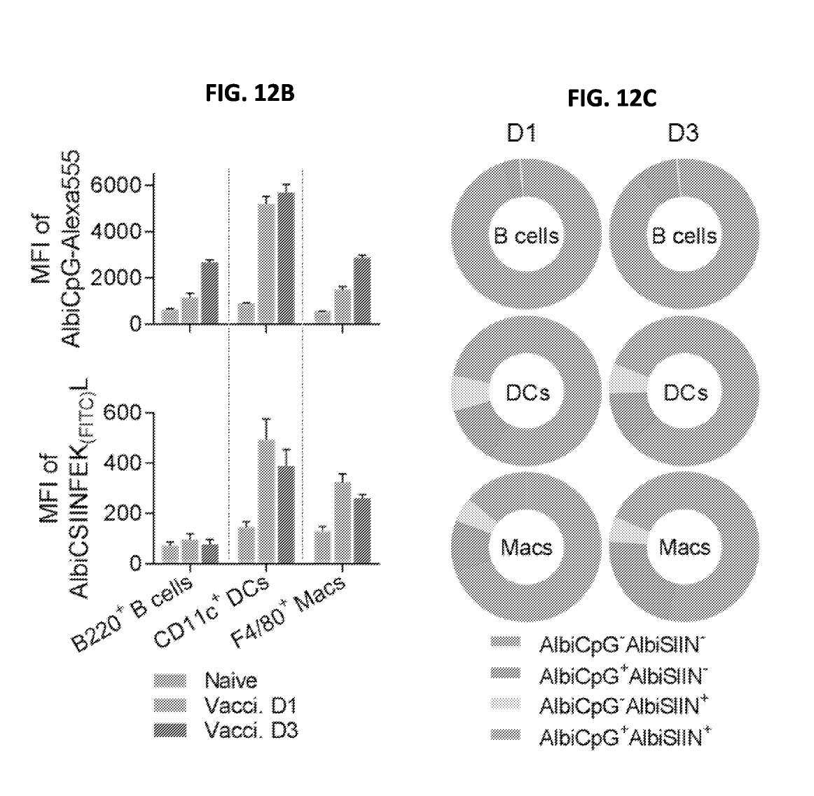

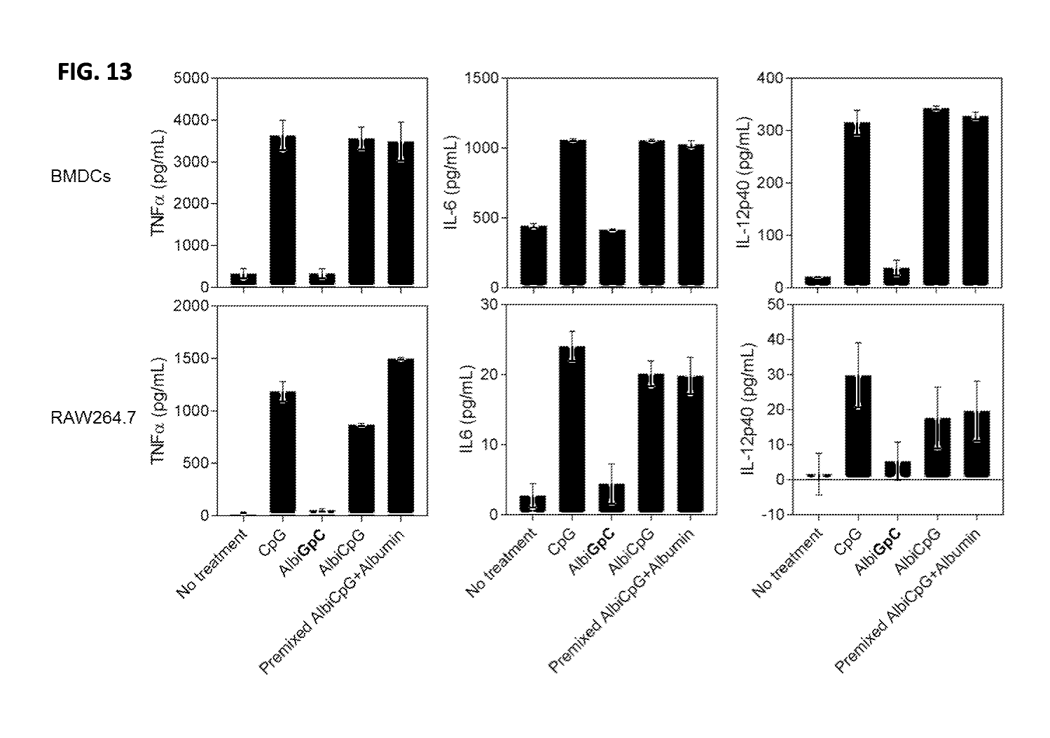

The co-delivery of CpG and antigens via AlbiVax was then studied. A peptide antigen, SIINFEKL (SEQ ID NO: 84) (epitope of ovalbumin) was utilized, and also a modified SIINFEKL (SEQ ID NO: 84) with cysteine for MEB conjugation and with FITC on lysine which retained epitope binding ability to H-2K^{b} MHC class I molecules. The resulting MEB-CSIINFEK_{(FITC)}L (denoted as AlbiCSIINFEK_{(FITC)}L, SEQ ID NO: 85) was s.c. co-injected with AlbiCpG-Alexa555 at the tail base of C57BL/6 mice. After 1 and 3 days, it was found that IN LN-residing DCs and macrophages, but not B cells, showed relatively high uptake per cell (median fluorescence intensity, MFI) of both AlbiCpG and AlbiCSIINFEKL (SEQ ID NO: 85) (FIG. 12A-12B), with 15-20% of double-positive DCs and macrophages (AlbiCpG⁺ AlbiCSIINFEKL⁺) (SEQ ID NO: 85) (FIG. 12C), demonstrating intracellular co-delivery of CpG and peptide antigen to LN-residing APCs.

### Example 5

### In Vitro Immunostimulation of APCs by AlbiVax

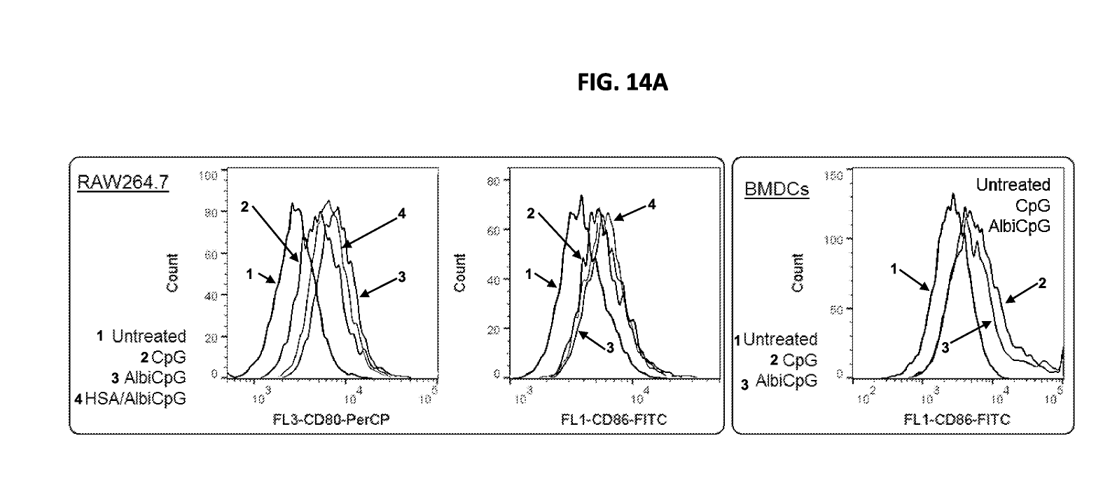

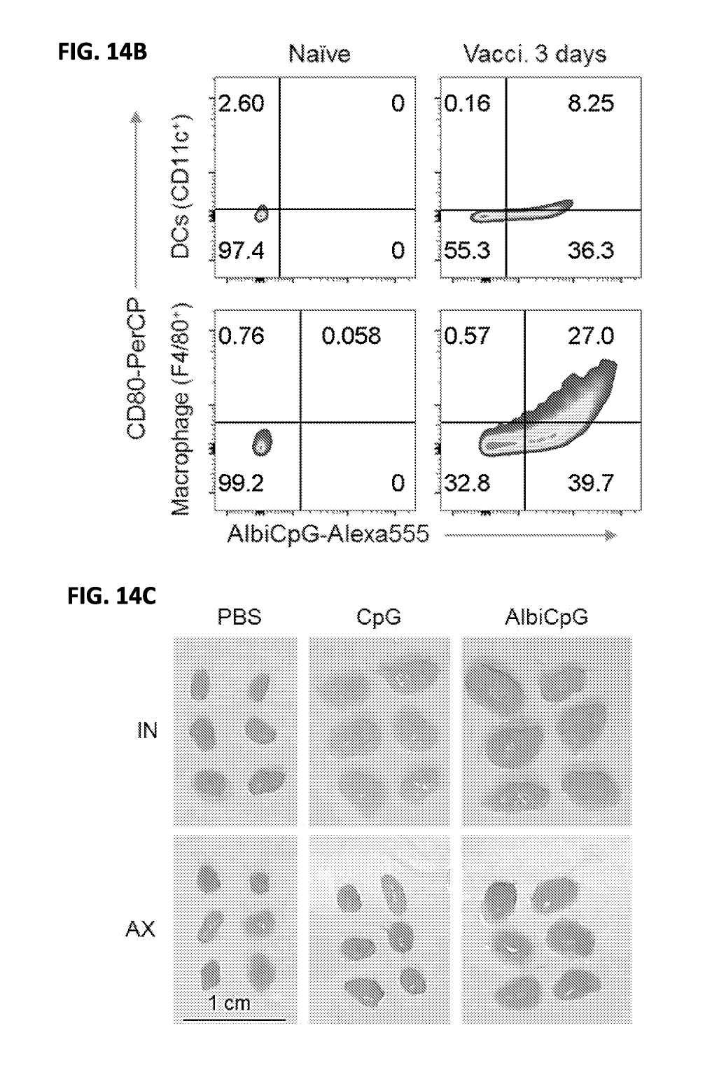

As a potent adjuvant, CpG stimulates APCs which phenotypically results in elevated secretion of proinflammatory cytokines, including tumor necrosis factor α (TNFα), interleukin 6 (IL-6), and interleukin 12 (IL-12), and also leads to the up-regulated expression of co-stimulatory factors. ELISA results indicated that AlbiVax induced dramatic secretion levels of proinflammatory factors in both BMDCs and RAW264.7 macrophages (FIG. 10), at a level comparable to CpG. Premixed AlbiVax-albumin complexes also induced secretion of comparable levels of proinflammatory factors, suggesting that conjugation of MEB and binding with albumin maintained the immunostimulatory activity of CpG. The control MEB-GpC did not stimulate these cells significantly more potently than the background (FIG. 10). The preservation of immunostimulation activity in AlbiVax, in which MEB was conjugated on the 3′ end of CpG, is consistent with previous reports (Putta et al., 2010, Bioconjugate chemistry 21(1):39-45). The immunostimulatory activity of AlbiVax was further assessed by the expression levels of co-stimulatory factors. Particularly, after treatment with AlbiVax for 24 h, the expression levels of CD80 and CD86 were both elevated (FIG. 14A). Taken together, these results demonstrated that AlbiVax is a potent immunostimulator of APCs. When injected subcutaneously into C57BL/6 mice, AlbiCpG upregulated the expression of co-stimulatory factors CD80 on DCs in draining LNs (FIG. 14b), despite accompanied lymphadenopathy (FIG. 14C).

### Example 6

### AlbiVax Reduced Toxicity of CpG

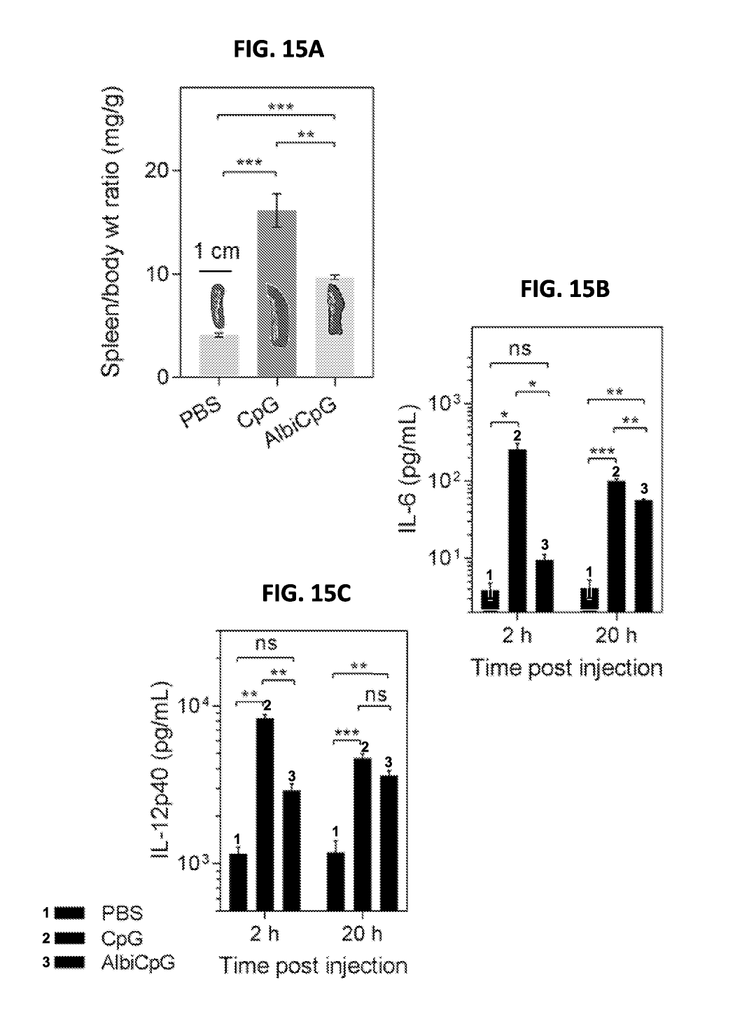

Clinical investigation has once been challenged by its toxicity, for which one primary causes is the systemic inflammation induced by CpG in the systemic circulation. To address this safety problem, AlbiVax was designed to reduce the systemic toxicity by prolong the retention of CpG in LNs and prolong CpG release. Further, after treating mice with free CpG or AlbiVax at 5 nmole/mouse on day 0 and day 3, free CpG induced much more serious splenomegaly than AlbiVax, as analyzed on day 6. There was more lymphadenopathy in mice treated with AlbiVax than that of free CpG (FIG. 15A). However, this is an indication that AlbiVax induced more potent immunostimulation by immune cell proliferation or recruitment into LNs. C57BL/6 mice were treated with AlbiVax or free CpG (5 nmole/mouse), followed by assaying the serum concentrations of proinflammatory factors. Compared with CpG, AlbiCpG caused significantly lower elevation of blood proinflammatory factors, IL-6 and IL-12p40, at 2 h post vaccination (FIG. 15B, 15C). At 20 h post injection, the concentrations of IL-6 and IL-12p40 also increased in AlbiCpG-treated mice due to immunostimulation (FIGS. 15B, 15C).

### Example 7

### AlbiVax Elicited Potent and Durable T Cell Response

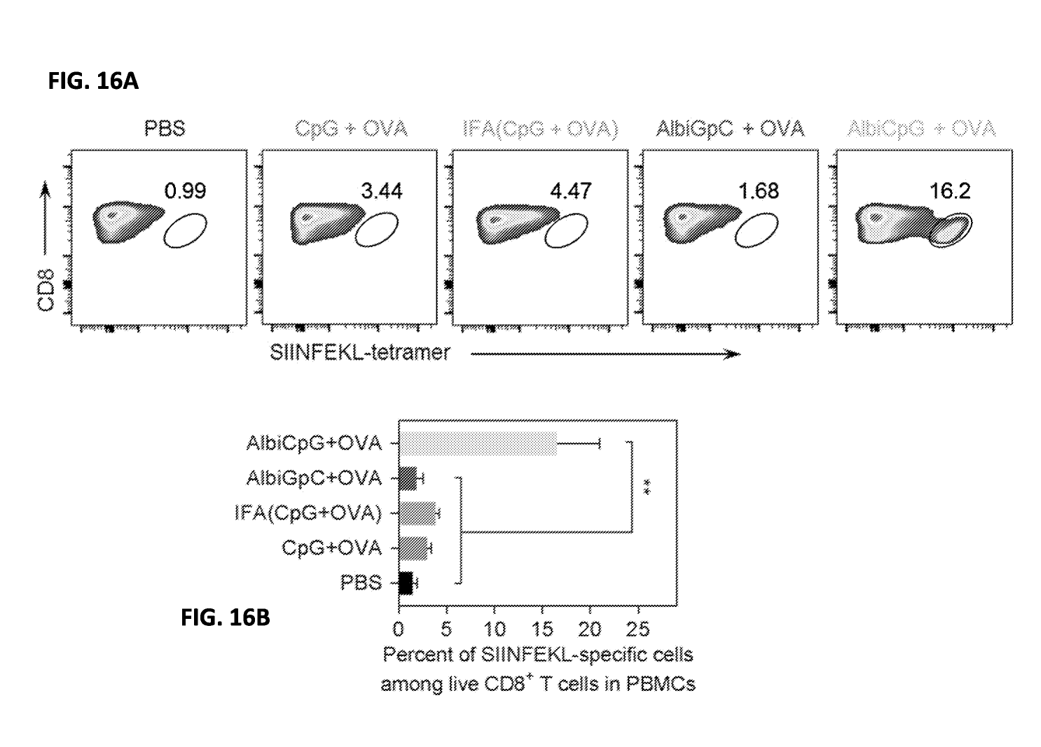

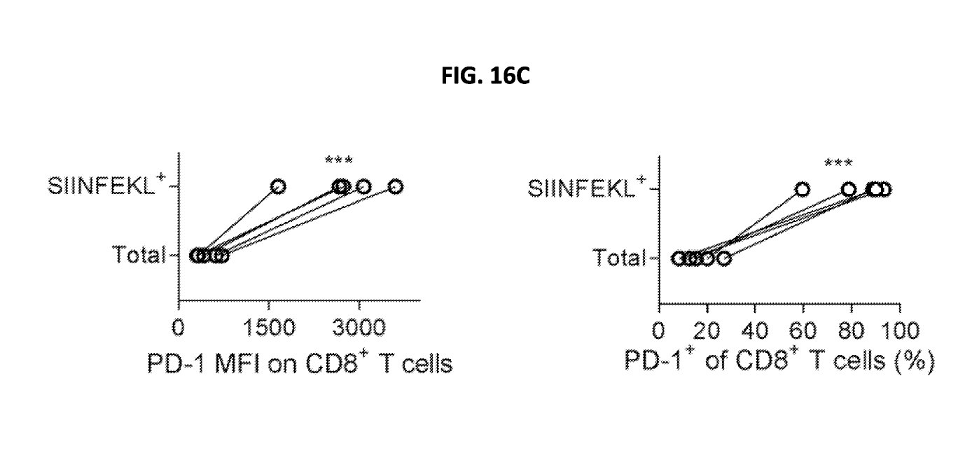

With efficient intranodal and APC intracellular delivery, AlbiCpG were then studied for T cell response together with ovalbumin (OVA) antigen. C57BL/6 mice were immunized with 2 nmole AlbiCpG and 10 μg OVA on day0 and day14, and CD8⁺ T cells in peripheral blood were stained using a H-2K^{b}-SIINFEKL (SEQ ID NO: 84) tetramer on day21(FIG. 16A). The frequencies of SIINFEKL (SEQ ID NO: 84) tetramer⁺ CD8⁺ T cells was 2.9±0.2% induced by CpG+OVA, 3.8±0.2% by CpG+OVA emulsified in IFA despite its benchmark clinical use, and in contrast, 16.5±2.56% by AlbiCpG+OVA (p<0.01); no significant T cell response was induced by control AlbiGpC+OVA, indicating low immunogenicity of MEB moiety. In mice immunized with AlbiCpG+OVA, 16.7±3.2% of total CD8⁺ T cells expressed immune checkpoint PD-1; among SIINKFEKL (SEQ ID NO: 84)-specific CD8⁺ T cells, the frequency of PD-expressing increased to 86.1±6.1%, and the PD-1 MFI was 5-fold higher than that on total CD8⁺ T cells (FIG. 16B). The upregulated PD-1 expression upon AlbiVax-elicited T cell activation suggests chronic antigen stimulation that eventually causes T cell exhaustion, and compared with total CD8⁺ T cells, the differential phenotypic characteristics of Ag-specific CD8⁺ T cells upon vaccination indicate that Ag-specific CD8⁺ T cells exhibit a more exhausted state, in line with clinical observations.

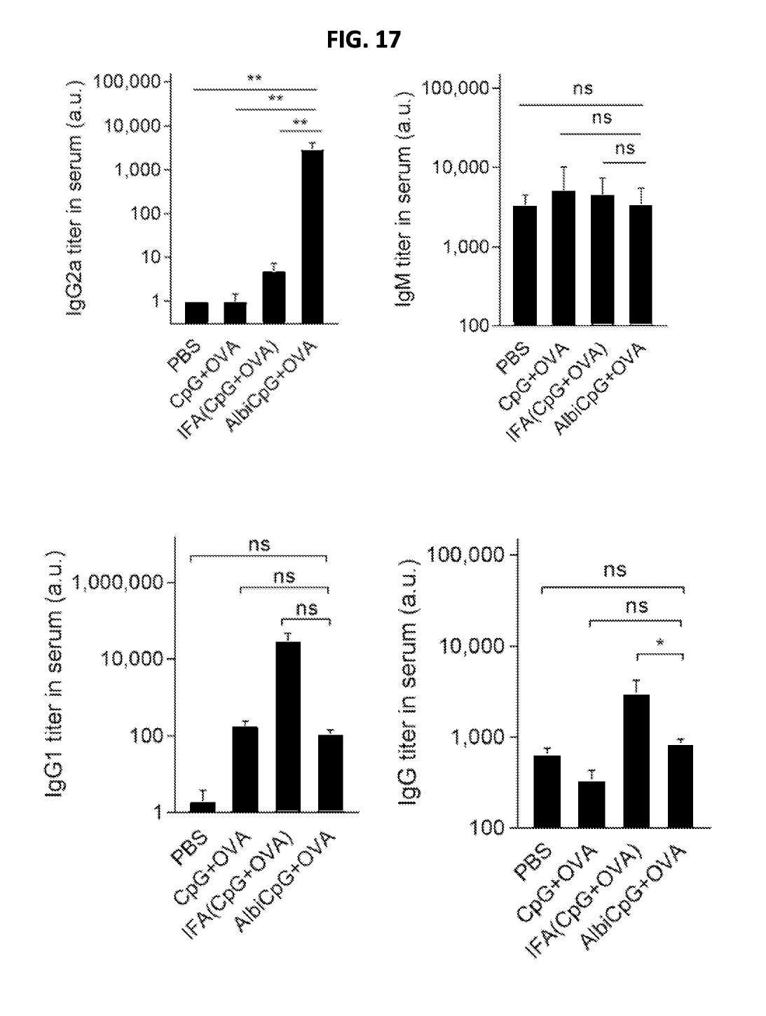

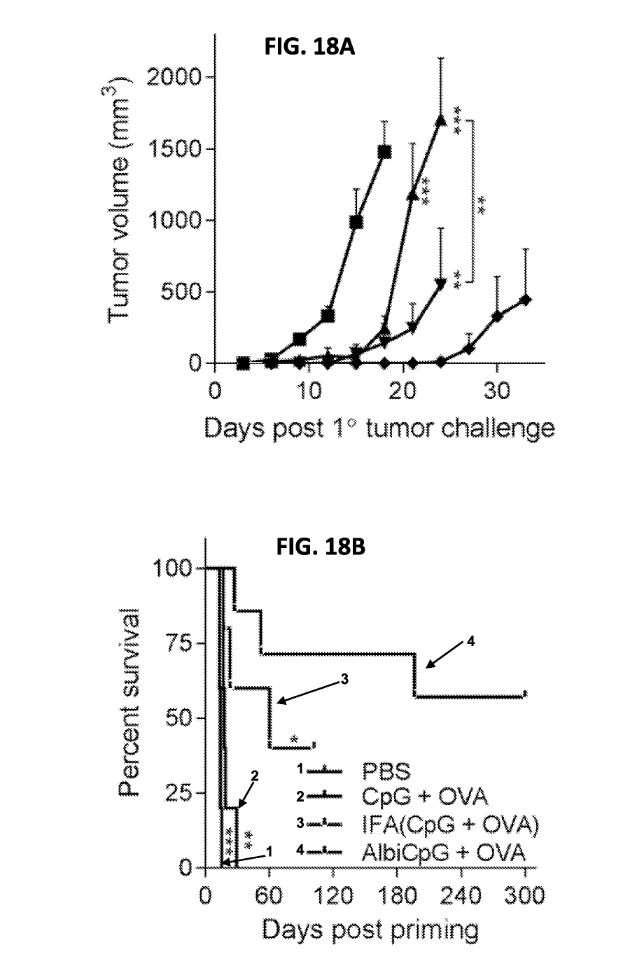

Given the low spontaneous T cell response in many patients and hence the low to medium response rate of immune checkpoint blockade, these observations provide an opportunity to elicit Ag-specific T cell response and enhance the response rate to checkpoint inhibitors such as anti-PD-1 by combining anti-PD-1 and AlbiVax for optimal therapeutic efficacy. AlbiCpG+OVA also potentiated the secretion of Ag-specific IgG2a which benefits cancer therapy (FIG. 17). On day 71, immunized mice were s.c. challenged with 3×10⁵ EG7.OVA cells. Marginal survival benefits were observed in mice vaccinated with CpG+OVA, despite early delay of tumor progression; IFA(CpG+OVA) moderately protected against tumor challenge; in contrast, AlbiCpG+OVA potently protected mice from challenging, with 71% (5/7) mice remaining tumor-free for >3 months (FIG. 18A, 18B). The surviving mice were re-challenged s.c. with 3×10⁵ EG7.OVA cells on day120, and all these mice survived the 2° challenge for >3 months owing to durable T cell responses elicited by AlbiVax.

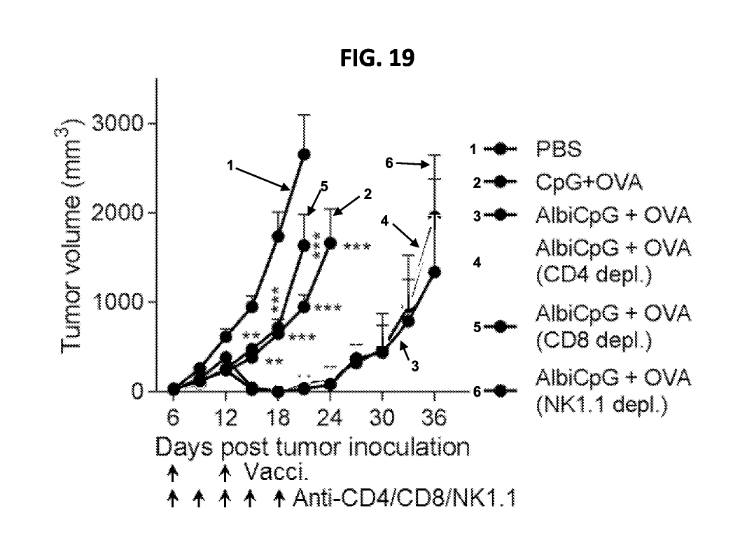

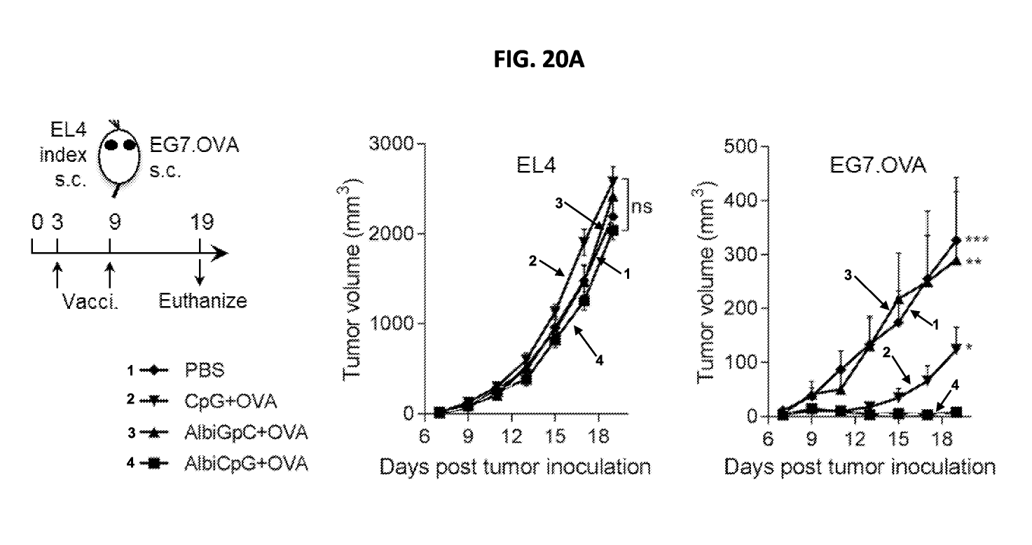

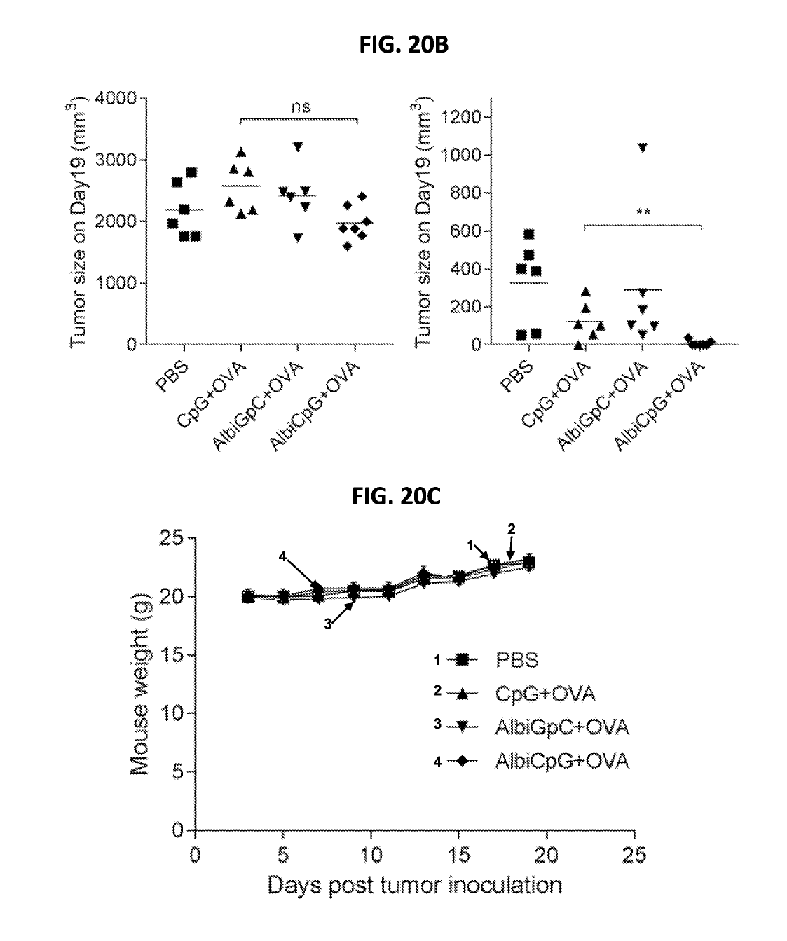

AlbiCpG+OVA was then investigated for immunotherapy of established tumors. C57BL/6 mice were s.c. inoculated with 3×10⁵ EG7.OVA, and on day6 post inoculation (˜35 mm³ tumor), mice were treated with AlbiCpG+OVA, followed by boosting on day12 (2 nmol CpG, 20 μg OVA). While CpG+OVA moderately retarded tumor progression, AlbiCpG+OVA markedly regressed tumor upon boosting in 5/8 mice by day24 (FIG. 19). While depletion of CD4 T cells or natural killer 1.1 (NK1.1) cells marginally affected the therapeutic outcome of AlbiCpG+OVA, depletion of CD8 T cells markedly neutralized its efficacy, suggesting the central role of CD8⁺ CTLs in AlbiVax-based immunotherapy (FIG. 19). Further, C57BL/6 mice were s.c. inoculated with 3×10⁵ EL4 cells (OVA-) on the left shoulder and 3×10⁵ EG7.OVA on the right shoulder, and again treated with AlbiCpG+OVA. While EL4 tumor progression was marginally influenced, AlbiCpG+OVA again eradicated 4/6 EG7.OVA tumors by day19 (FIGS. 20A, 20B), thus confirming the Ag-specificity of AlbiVax-mediated immunotherapy. Moreover, no significant morbidity or toxicity was observed in AlbiVax-treated mice (FIG. 20C).

### Example 8

### AlbiVax-Based Combination Melanoma Immunotherapy

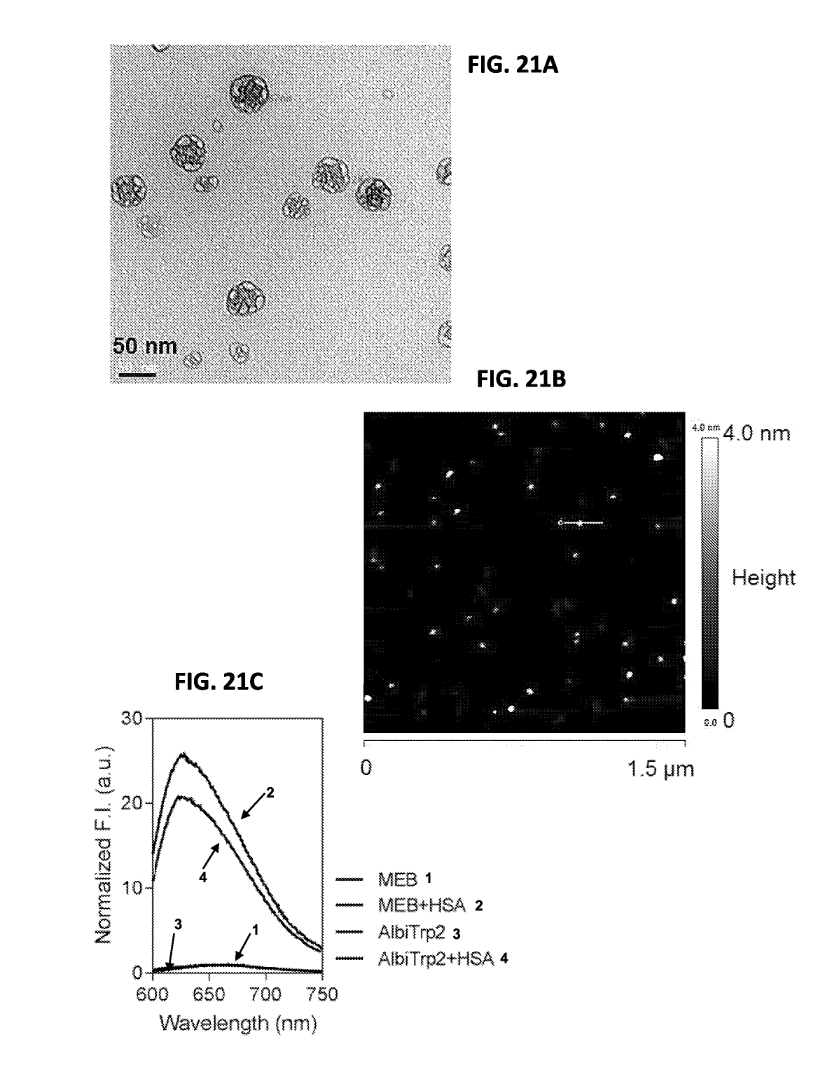

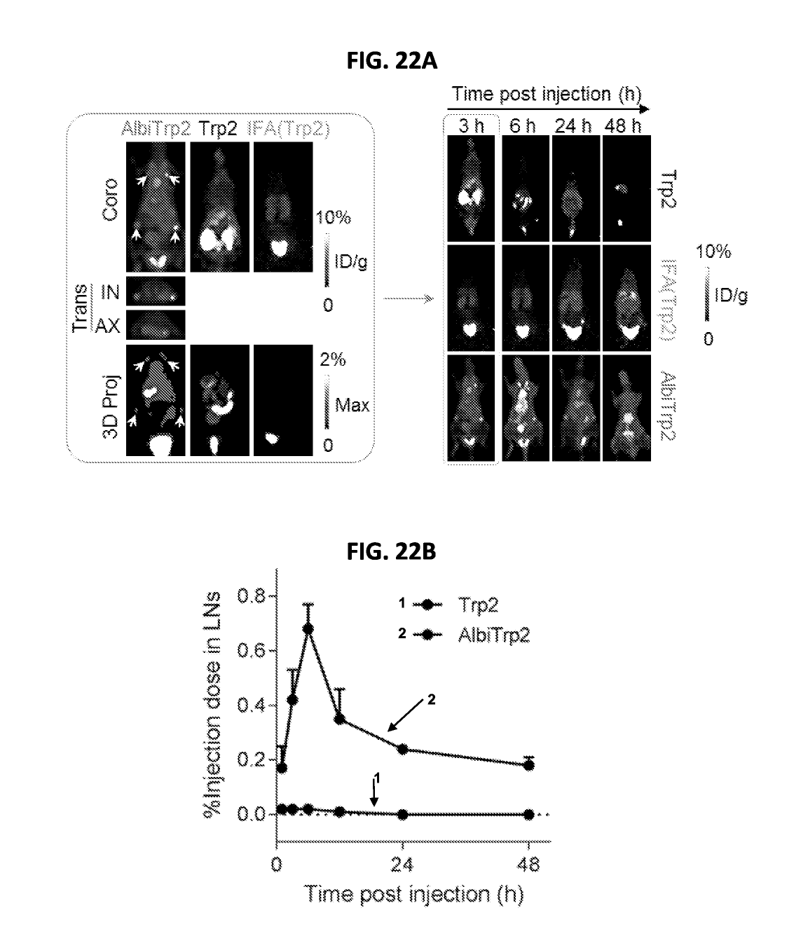

Encouraged by the potent and durable T cell responses elicited by AlbiVax, AlbiVax was studied for immunotherapy of melanoma, a type of aggressive skin tumor whose clinical outcome can be improved by potentiating antitumor immunity. Tyrosinase-related protein 2 (Trp2), a melanoma-associated subunit Ag, was used in this study. Trp2 with an N-terminal cysteine was conjugated with MEB to prepare albumin-binding MEB-Trp2 (denoted as AlbiTrp2). Hydrophilic MEB increased the water solubility of AlbiTrp2 relative to Trp2, and the resulting amphiphilic AlbiTrp2 self-assembled into nanoparticles in aqueous solution (FIG. 21A, 21B). AlbiTrp2 was able to tightly bind with albumin (K_{d}=0.79 μM. R²=0.88), which drove the transformation from AlbiTrp2 amphiphilic nanoparticles to albumin/AlbiTrp2 complexes and recovered MEB fluorescence (FIG. 21c). To study LN-targeted Trp2 delivery, ⁶⁴Cu-labeled AlbiTrp2 was s.c. injected at the tail base of FVB mice for PET imaging (FIG. 22A). While Trp2 was systemically disseminated rapidly, leaving nondetectable Trp2 in draining LNs, AlbiTrp2 was efficiently delivered to LNs, with 91-fold larger AUC in LNs within 3 days than free Trp2 (FIG. 22B); in contrast, IFA-emulsifying Trp2 dramatically retained Trp2 in the injection sites, which could not only sequestrate but also disarm and delete Ag-specific CD8⁺ T cell.

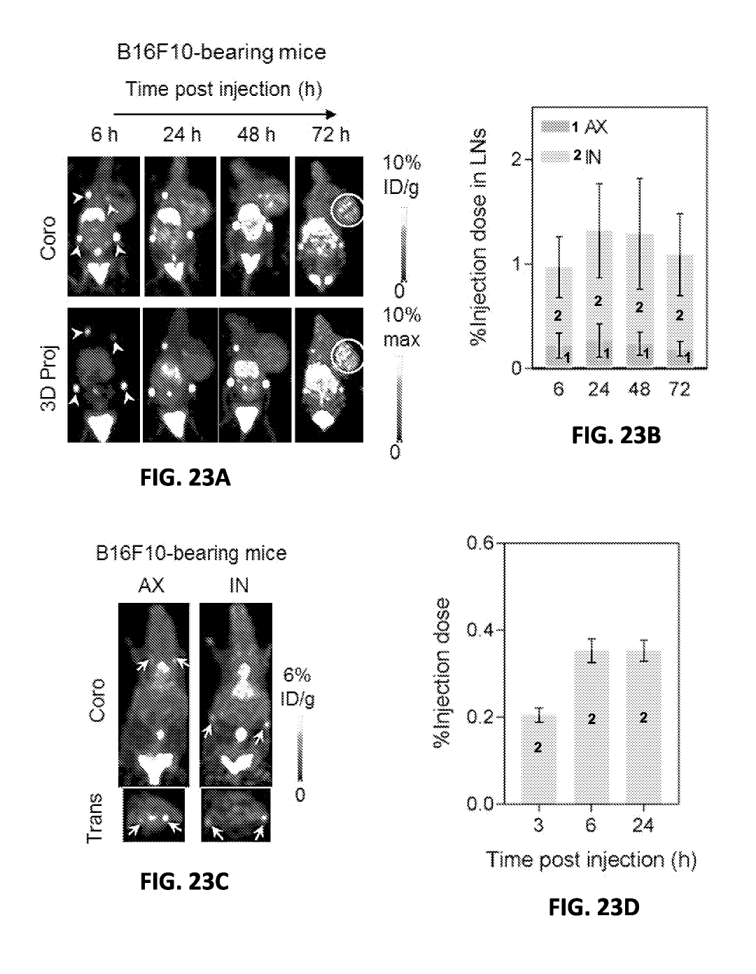

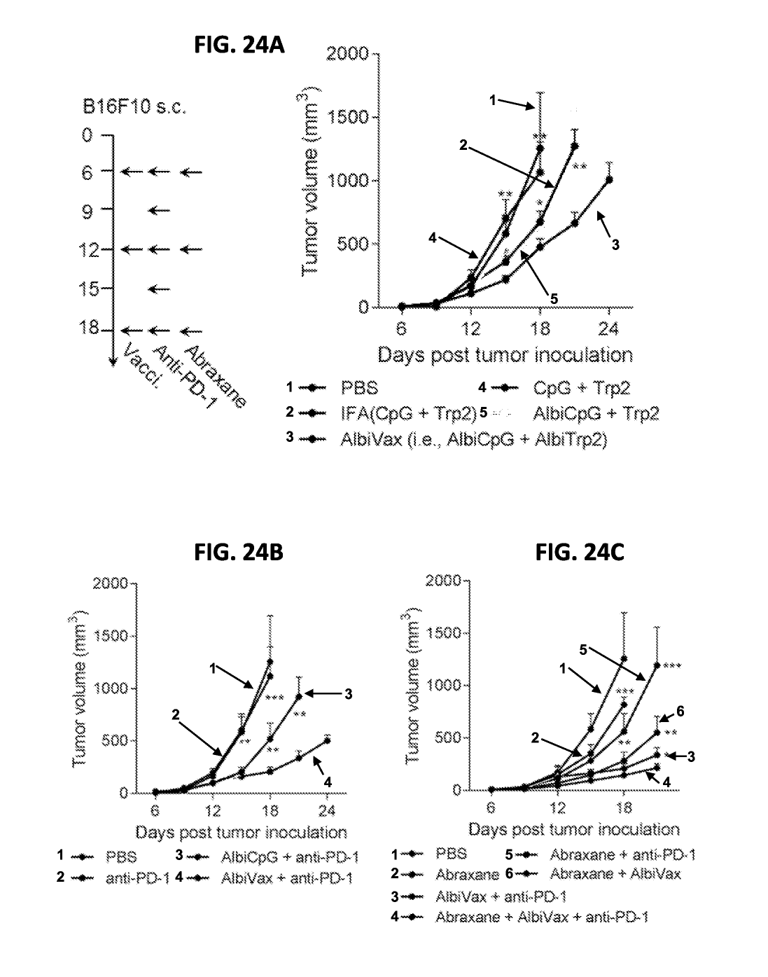

AlbiCpG+AlbiTrp2 was then investigated for melanoma immunotherapy in B16F10-tumor-bearing syngeneic C57BL/6 mice, in which it was verified by PET imaging that both AlbiCpG (FIGS. 23A, 23B) and AlbiTrp2 (FIGS. 23C, 23D) were efficiently delivered to LNs except tumor draining axillary LNs, due to damaged lymphatic drainage in tumor. For immunotherapy, C57BL/6 mice s.c. inoculated with 3×10⁵ B16F10 cells were treated with AlbiCpG (2 nmole CpG equivalents)+AlbiTrp2 (20 μg) on days 6, 12, and 18 post inoculation. While none of CpG+Trp2, AlbiCpG+Trp2, and IFA(CpG+Trp2) significantly inhibited tumor progression, AlbiCpG+AlbiTrp2 significantly prohibited tumor growth (FIG. 24A). Double combination of anti-PD-1 and AlbiCpG+AlbiTrp2 enhanced the therapeutic efficacy (FIG. 24B). Further, triple combination of anti-PD-1, AlbiCpG+AlbiTrp2, and chemotherapy Abraxane (albumin-bound paclitaxel) further inhibited the tumor progression (FIG. 24C). Even though no tumors were eradicated, enhanced therapeutic efficacy can be achieved by using a multi-epitope AlbiVax to induce a broader spectrum of antitumor T cell responses. Furthermore, using a combination of multiple synergistic therapeutics can be used. Taking advantage of the systemic T cell responses induced by AlbiVax, AlbiCpG+AlbiTrp2 was investigated for the immunotherapy of metastatic melanoma, which is a significant death cause of melanoma patients.

### Example 9

### Neoantigen-Based AlbiVax for Personalized Tumor Immunotherapy

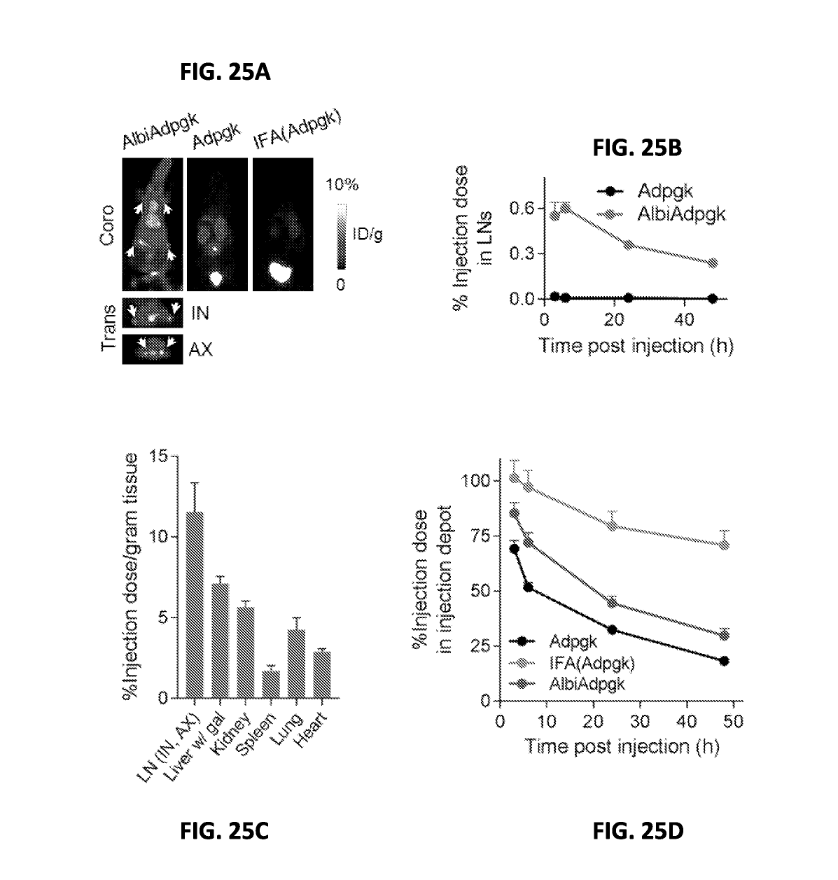

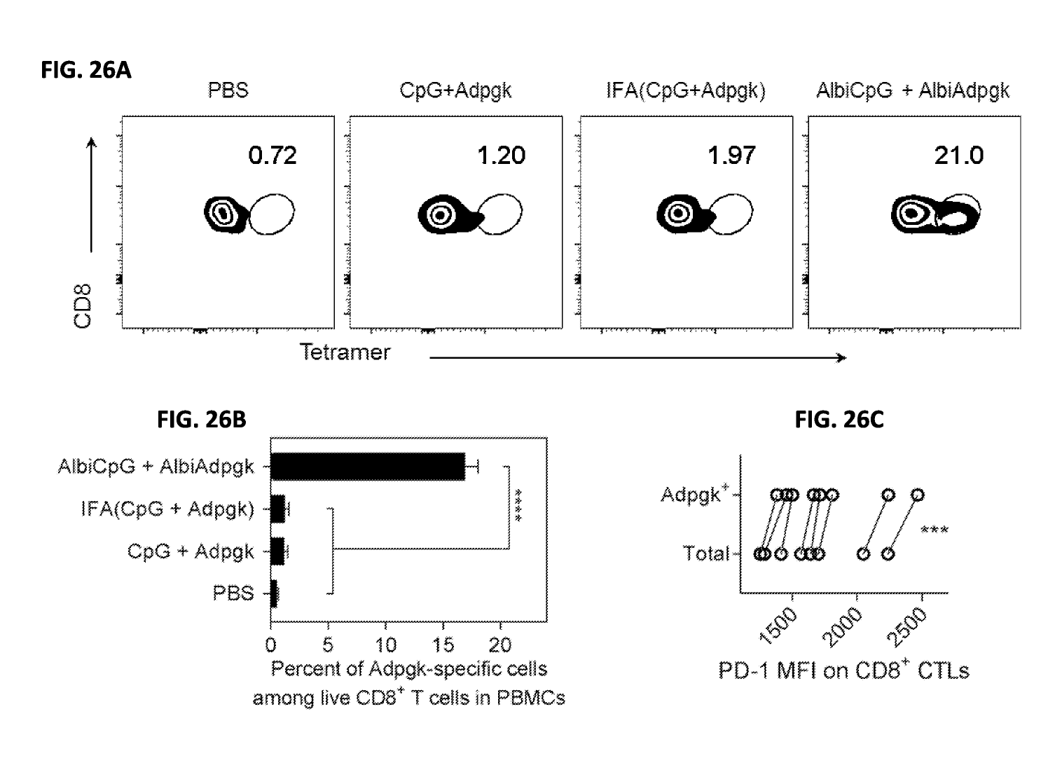

Encouraged by the efficient delivery of peptide antigen, AlbiVax-based neoantigen delivery was exploited for personalized tumor immunotherapy. Adpgk, a neoantigen [ASMTN(R→M)ELM] (SEQ ID NO: 86) in MC38 (colon cancer) tumor cells⁷ was used. The conjugation of hydrophilic MEB improved the water solubility of MEB-Adpgk (denoted as AlbiAdpgk). Using ⁶⁴Cu-labeled NMEB-Adpgk, PET imaging again demonstrated efficient LN-targeted delivery of AlbiAdpgk, and the AUC in IN and AX LNs was 43-fold higher than that of Adpgk or IFA(Adpgk) within 2 days (FIG. 25A-25C). Significantly less AlbiAdpgk was trapped at the injection sites than IFA(Adpgk) (FIG. 25D). Immunization of C57BL/6 mice with AlbiVax (AlbiCpG+AlbiAdpgk) on day0 and 14 elicited 14.1- and 13.6-fold greater frequency of peripheral Adpgk-specific CD8⁺ CTLs than CpG+Adpgk and IFA(CpG+Adpgk), respectively, as stained using a H-2D^{b}-ASMTNMELM (SEQ ID NO: 86) tetramer (FIG. 26A, 26B). AlbiCpG+AlbiAdpgk upregulated PD-1 expression on peripheral CD8⁺ CTLs, especially Adpgk⁺ CD8⁺ CTLs (FIG. 26C).

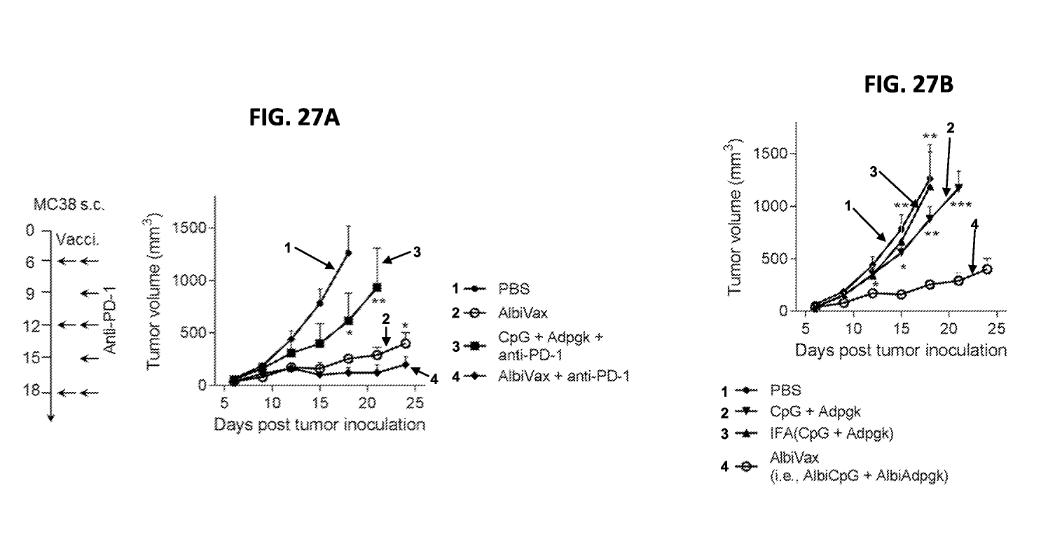

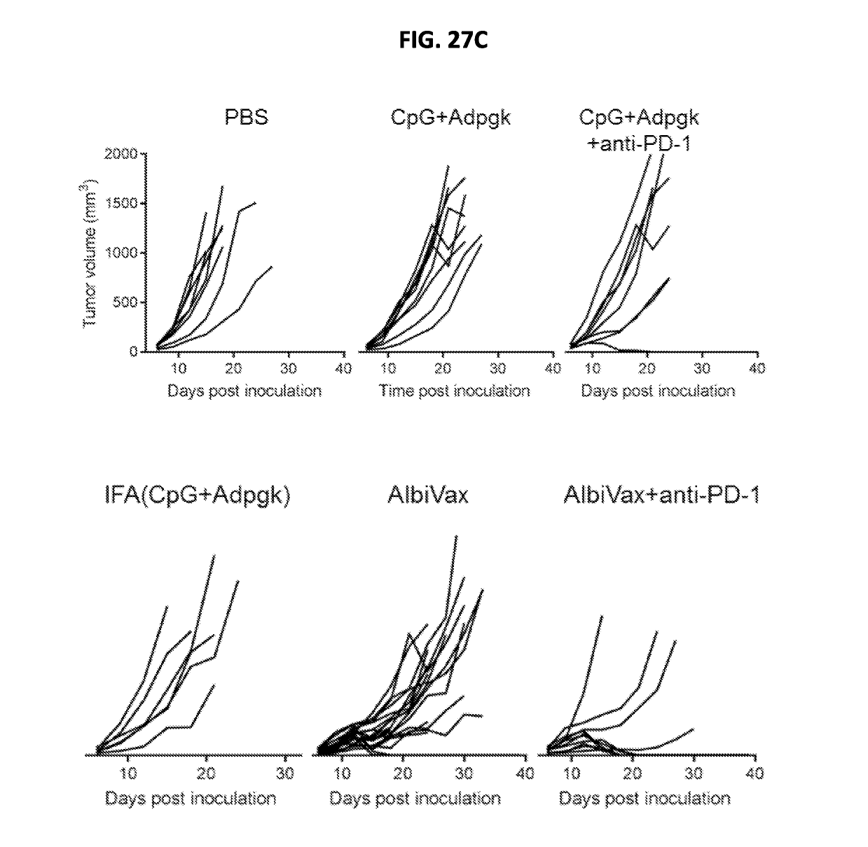

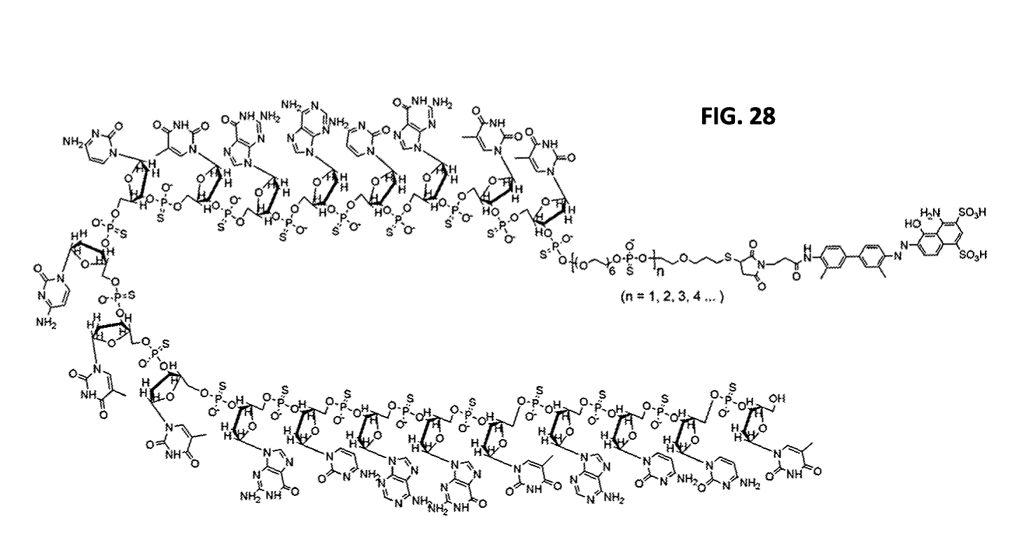

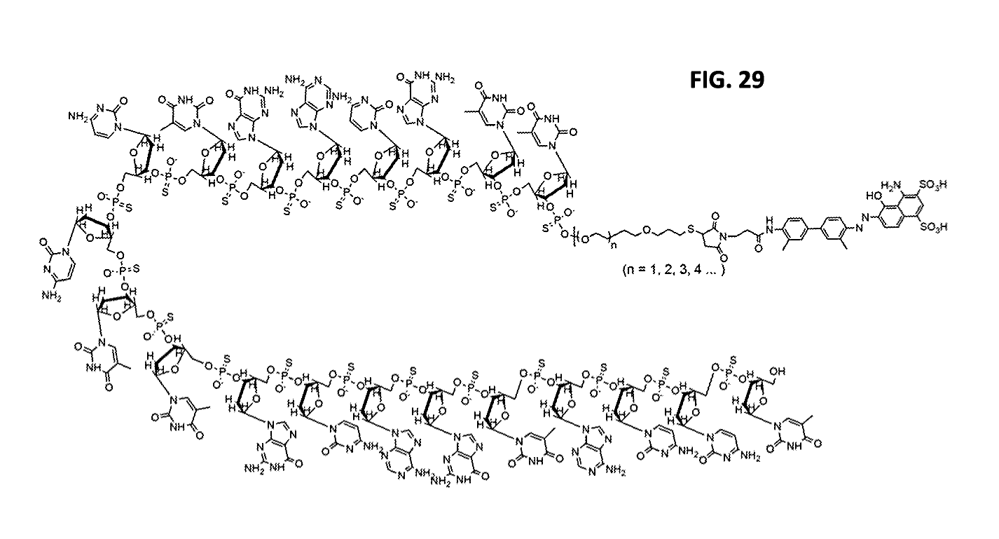

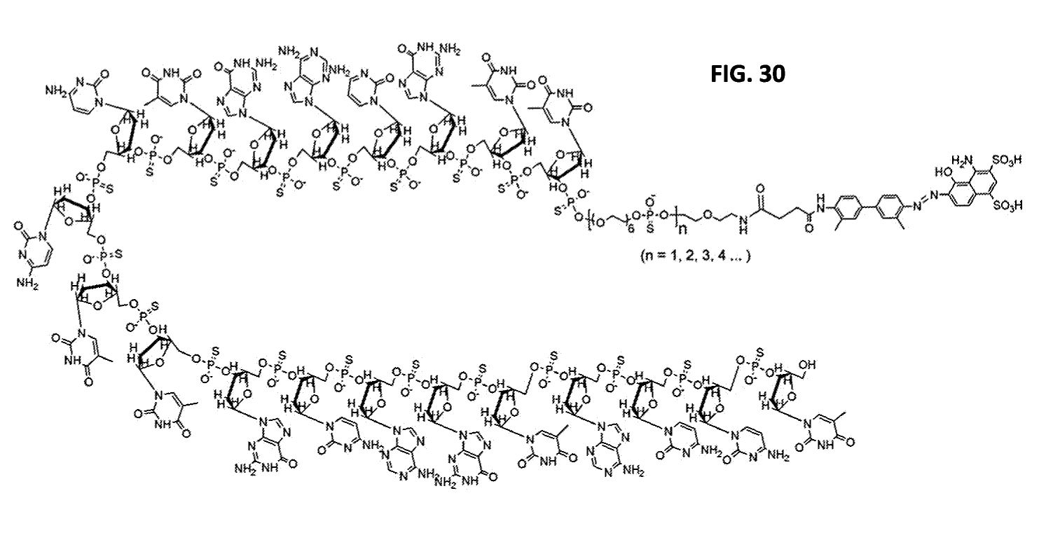

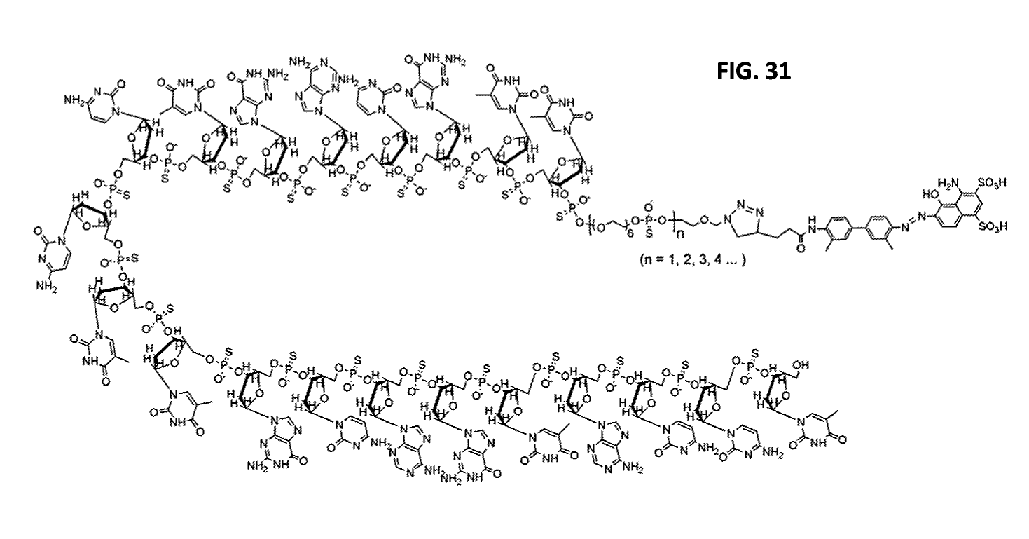

To study AlbiCpG+AlbiAdpgk for personalized tumor immunotherapy of established tumor, 3×10⁵ MC38 cells were s.c. inoculated in C57BL/6 mice, and treatment was initiated on day6 (tumor volume ˜30 mm³). In contrast to the moderate inhibition of tumor progression by IFA(CpG+Adpgk), AlbiCpG+AlbiAdpgk inhibited tumor progression significantly more effectively and regressed 2/16 tumors (FIG. 27A, 27C). By combining anti-PD-1 with AlbiCpG+AlbiAdpgk, CD8⁺ CTLs reinvigoration markedly increased the response rate and led to complete regression of 6/10 tumors for >60 days (FIG. 6B, 6C).

1. A chemical conjugate having a structure satisfying Formula I 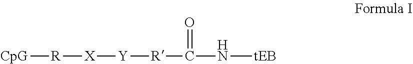
wherein
CpG is a CpG oligodeoxynucleotide;
tEB is a truncated Evans Blue dye;
X is selected from amine, sulfur, carbonyl, or hydroxy amine;
Y is selected from ester, amine, aliphatic, or a pyrrolidine dione; or
X and Y combine to form a triazole or cyclooctatriazole; and
each of R and R′ independently is selected from an aliphatic linker, an amide linker, an alkylene oxide linker, a peptide linker, or an oligonucleotide linker.

2. The chemical conjugate of claim 1, wherein the Evans Blue dye has a Formula II 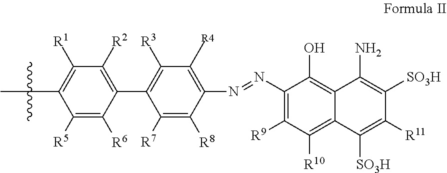
wherein each of R¹, R², R³, R⁴, R⁵, R⁶, R⁷, R⁸, R⁹, R¹⁰, and R¹¹ independently is selected from hydrogen, halogen, hydroxyl, cyano, aliphatic, heteroaliphatic, haloaliphatic, or haloheteroaliphatic.

3. The chemical conjugate of claim 2, wherein R¹ and R⁴ are each selected independently from halogen, hydroxyl, cyano, C₁-C₆alkyl, C₁-C₆alkoxy, C₁-C₆haloalkyl, or C₁-C₆haloalkoxy.

4. The chemical conjugate of claim 2, wherein R², R³, R⁵, R⁶, R⁷, R⁸, R⁹, R¹⁰, and R¹¹ are each hydrogen.

5. The chemical conjugate of claim 2, wherein R¹ and R⁴ are each C₁-C₆alkyl.

6. The chemical conjugate of claim 2, wherein R¹ and R⁴ are each methyl.

7. The chemical conjugate of claim 1, wherein X is selected from —NR^{a}—, wherein R^{a} is selected from hydrogen, aliphatic, or aryl; sulfur; —C(O)—; or

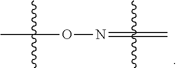 [SMILES: *ON=*]

8. The chemical conjugate of claim 1, wherein Y is selected from —C(O)O—; —NR^{a}—, wherein R^{a} is selected from hydrogen, aliphatic, or aryl;

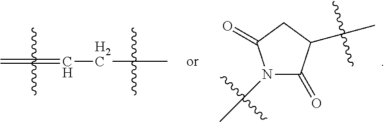

9. The chemical conjugate of claim 1, wherein X and Y combine to form a triazole having a structure 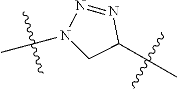 [SMILES: *C1CN(*)N=N1]
or a cyclooctatrizazole having a structure

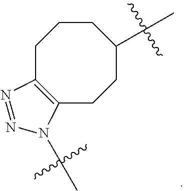 [SMILES: *C1CCCc2nnn(*)c2CC1]

10. The chemical conjugate of claim 1, wherein X is amine and Y is carboxyl.

11. The chemical conjugate of claim 1, wherein X is sulfur and Y is

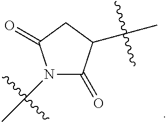 [SMILES: *C1CC(=O)N(*)C1=O]

12. The chemical conjugate of claim 1, wherein X is a carbonyl group and Y is an amine.

13. The chemical conjugate of claim 1, wherein X is a hydroxy amine and Y is alkyl.

14. The chemical conjugate of claim 13, wherein X and Y combine to form an oxime group having a structure

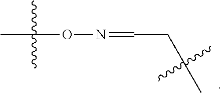 [SMILES: *CC=NO*]

15. The chemical conjugate of claim 1, wherein one or both of R and R′ is an oligonucleotide linker having a formula selected from (A)ₙ, (T)ₙ, (C)ₙ, (G)ₙ, (AT)ₙ, (AC)ₙ, (AG)ₙ, (TC)ₙ, (ATC)ₙ, (ATCG)ₙ, or (ATCGA)ₙ; wherein each n is selected from an integer that provides a linker length of greater than 0 nm to 200 nm.

16. The chemical conjugate of claim 1, wherein one or both of R and R′ is a peptide linker having a formula selected from (G)ₙ, (A)ₙ, (S)ₙ, (T)ₙ, (GA)ₙ, (TA)ₙ, (SA)ₙ, (GAT)ₙ, (TAS)ₙ, (SATA)ₙ, wherein each n is selected from an integer that provides a linker length of greater than 0 nm to 200 nm.

17. The chemical conjugate of claim 1, wherein one or both of R and R′ is an aliphatic linker selected from alkyl, alkenyl, alkynyl, or a combination thereof.

18. The chemical conjugate of claim 17, wherein the aliphatic linker is alkyl having a formula —(C(R^{b})₂)ₙ—, wherein each R^{b} independently can be hydrogen or aliphatic; and n is selected from an integer that provides a linker length of greater than 0 nm to 200 nm.

19. The chemical conjugate of claim 17, wherein the aliphatic linker is a C₁-C₂₀alkyl group.

20. The chemical conjugate of claim 1, wherein one or both of R and R′ is an alkylene oxide linker.

21. The chemical conjugate of claim 20, wherein the alkylene oxide linker is a PEG linker.

22. The chemical conjugate of claim 1, wherein one or both of R and R′ is an amide linker.

23. The chemical conjugate of claim 1, wherein R is an aliphatic linker and R′ is selected from an oligonucleotide linker, a peptide linker, an amide linker, or an alkylene oxide linker.

24. The chemical conjugate of claim 1, wherein R is an amide linker and R′ is selected from an aliphatic linker, an oligonucleotide linker, a peptide linker, or an alkylene oxide linker.

25. The chemical conjugate of claim 1, wherein R is an oligonucleotide linker and R′ is selected from a peptide linker, an aliphatic linker, an amide linker, or an alkylene oxide linker.

26. The chemical conjugate of claim 1, wherein R is a peptide linker and R′ is selected from an oligonucleotide linker, an aliphatic linker, an amide linker, or an alkylene oxide linker.

27. The chemical conjugate of claim 1, wherein R is an alkylene oxide linker and R′ is selected from an oligonucleotide linker, an aliphatic linker, an amide linker, or a peptide linker.

28. The chemical conjugate of claim 1, wherein the chemical conjugate has a structure satisfying any one or more of Formulas IV-IX 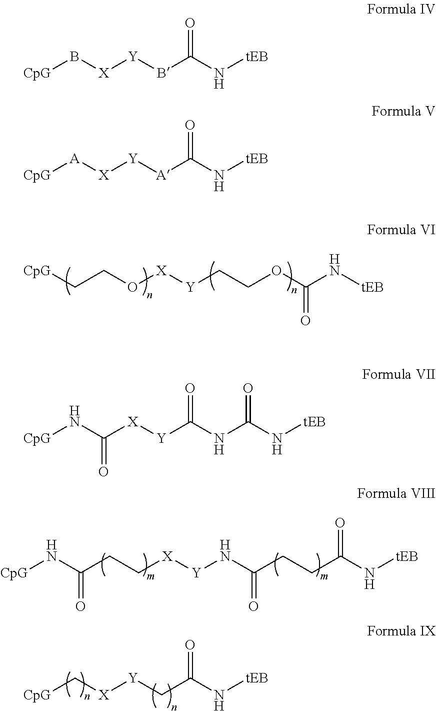
wherein
B and B′ independently are oligonucleotide linkers;
A and A′ independently are peptide linkers; and
each n independently is an integer that provides a linker length of greater than 0 to 200 nm.

29. The chemical conjugate of claim 1, wherein the chemical conjugate has a structure satisfying any one or more of the following formulas 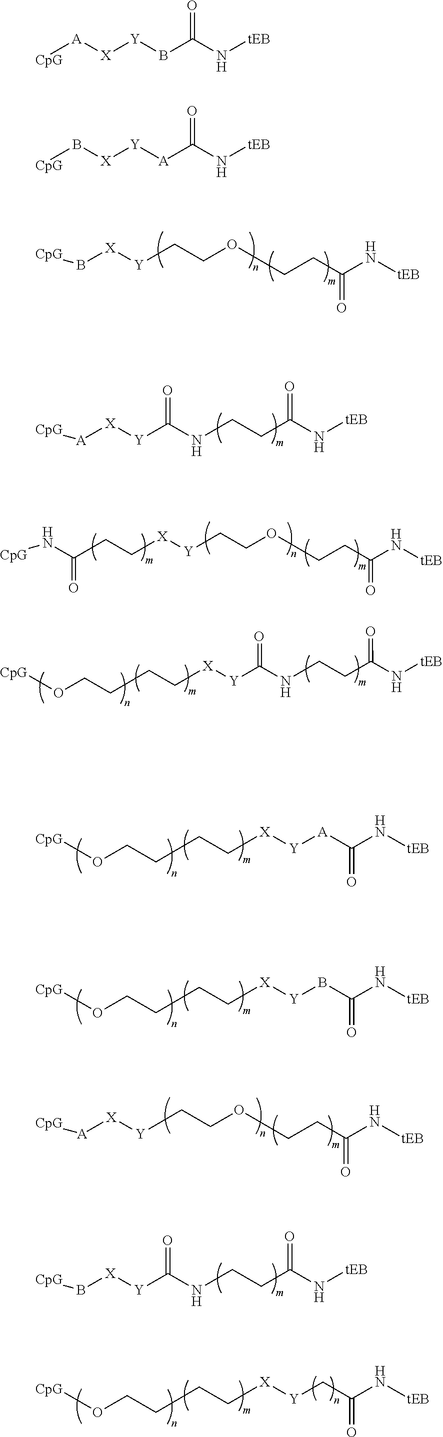 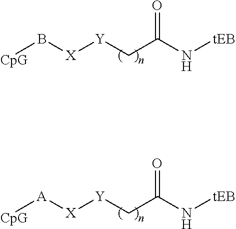
wherein
B is an oligonucleotide linker;
A is a peptide linker; and
each n independently is an integer that provides a linker length of greater than 0 to 200 nm.

30. The chemical conjugate of any claim 1, wherein the chemical conjugate has a structure satisfying any one of the following formulas

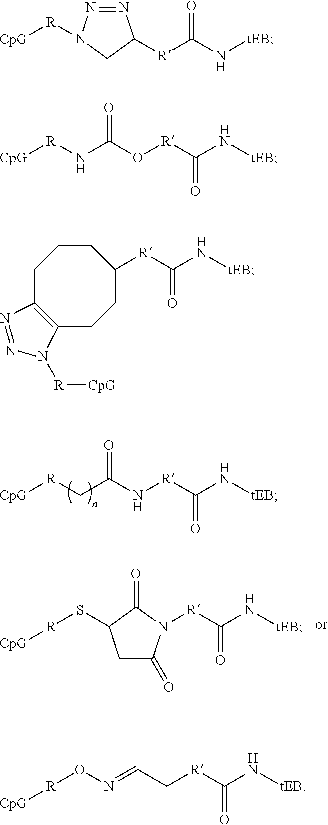

31. The chemical conjugate of claim 1, wherein the chemical conjugate has a structure satisfying any one of the following formulas

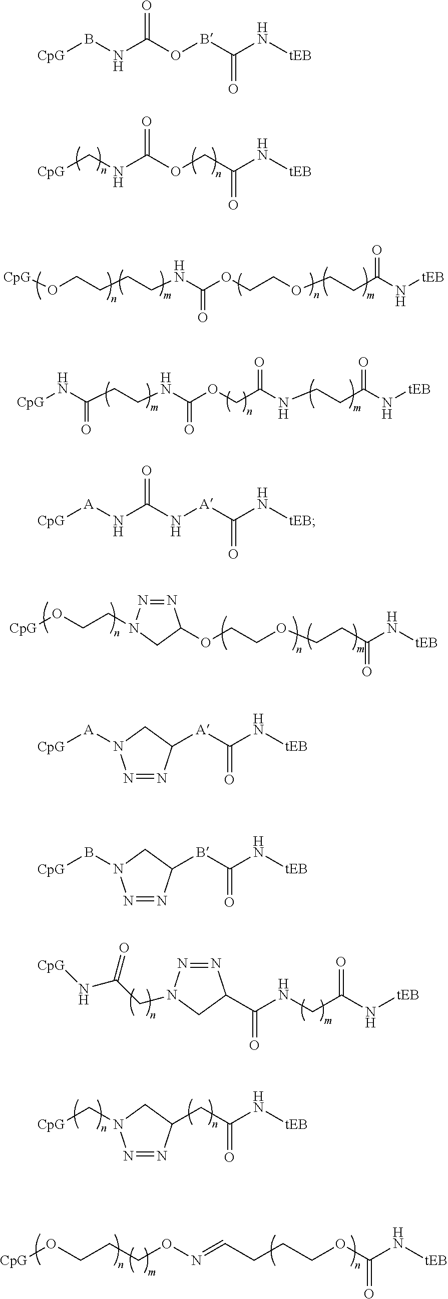

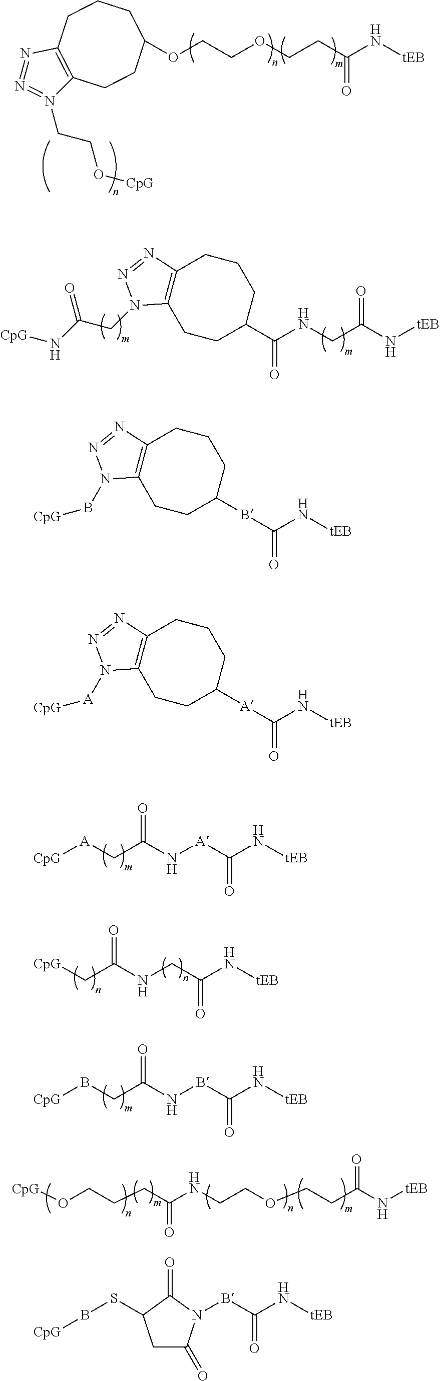

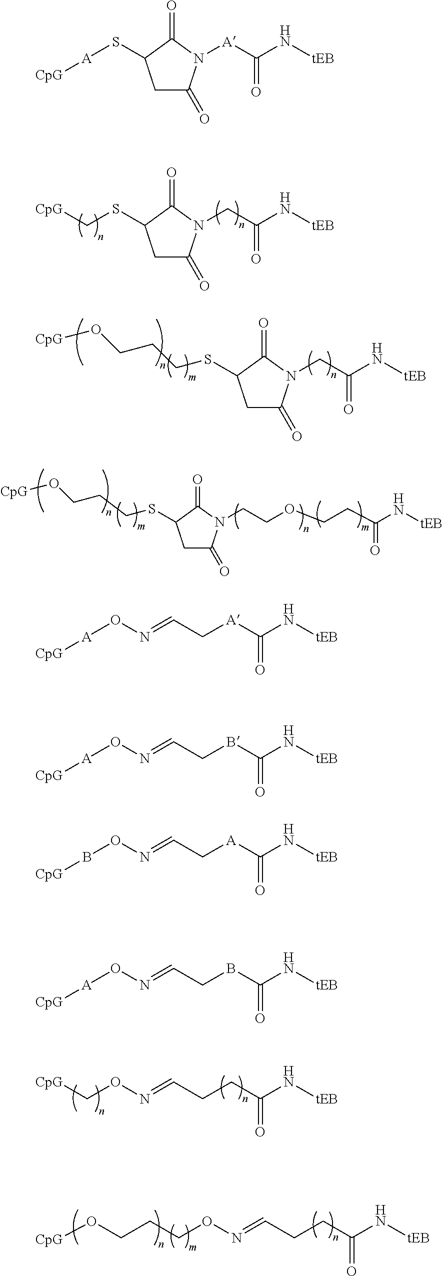

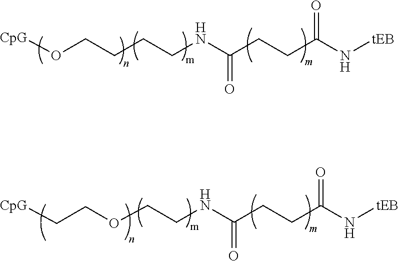

32. The chemical conjugate of claim 1, wherein the CpG group is attached to R through a phosphorothioate moiety, a PEG-functionalized phosphorothioate moiety, or polymeric PEG-functionalized phosphorothioate moiety.

33. The chemical conjugate of any one of claim 1, wherein the CpG group is attached to R through a —OP(S)(O⁻)O— group, a —Oₐ(CH₂)₂OP(S)(O⁻)O_{b}— group, wherein the CpG is bound to Oₐ and R is bound to O_{b}, or a —[Oₐ(CH₂)₂)ₙOP(S)(O⁻)O_{b}]_{n′}—, wherein the CpG is bound to Oₐ and R is bound to O_{b} and each of n and n′ independently can be 1 to 10, such as 1 to 8, or 1 to 6.

34. The chemical conjugate of claim 1, having a structure selected from

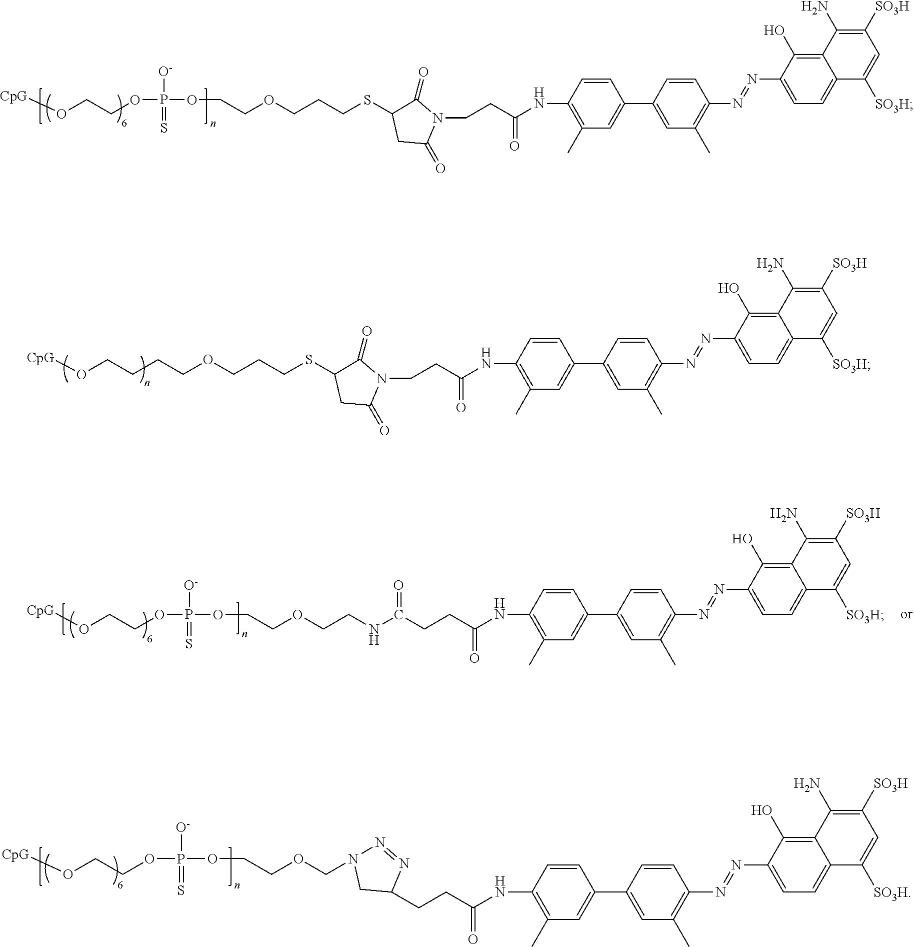

35. The chemical conjugate of claim 1, having a structure selected from

[symbol]

36. The chemical conjugate of claim 1, wherein the CpG oligodeoxynucleotide is a D-type, a K-type or a C-type CpG oligodeoxynucleotide.

37. The chemical conjugate of claim 36, wherein the K-type CpG oligodeoxynucleotide that has a nucleic acid sequence set forth as:
5′N₁N₂N₃D-CpG-WN₄N₅N₆ 3′ (SEQ ID NO: 1)
wherein the central CpG motif is unmethylated, D is T, G or A, W is A or T, and N₁, N₂, N₃, N₄, N₅, and N₆ are any nucleotide, wherein the CpG oligodeoxynucleotide is 10 to 30 nucleotides in length.

38. The chemical conjugate of claim 36, wherein the K-type CpG oligodeoxynucleotide that has a nucleic acid sequence set forth as one of SEQ ID NOs: 2-36.

39. The chemical conjugate of claim 36, wherein the D-type CpG oligodeoxynucleotide is least 18 nucleotides and no more than 30 nucleotides in length and comprises a sequence represented by the formula:
5′ X₁X₂X₃ Pu₁ Py₂ CpG Pu₃ Py₄ X₄X₅X₆(W)_{M} (G)_{N}-3′ (SEQ ID NO: 37)
wherein the central CpG motif is unmethylated, Pu is a purine nucleotide, Py is a pyrimidine nucleotide, X and W are any nucleotide, M is any integer from 0 to 10, and N is 4 to 8, wherein X₁X₂X₃ and X₄X₅X₆ are self complementary.

40. The chemical conjugate of claim 39, wherein Pu₁ Py₂ and Pu₃ Py₄ are self-complementary.

41. The chemical conjugate of claim 39, wherein the D-type CpG oligodeoxynucleotide comprises the nucleic acid sequence of one of SEQ ID NOs: 38-64.

42. The chemical conjugate of claim 36, wherein the C-type type CpG oligodeoxynucleotide comprises the nucleic acid sequence of one of SEQ ID NOs: 65-69.

43-65. (canceled)


SEQUENCE LISTING (88 sequences)

SEQ ID NO: 1 - DNA, 10 bp, Artificial Sequence
  CpG oligonucleotide
  misc_feature (1)..(3): N is any nucleotide
  misc_feature (8)..(10): N is any nucleotide
nnndcgwnnn

SEQ ID NO: 2 - DNA, 20 bp, Artificial Sequence
  CpG oligonucleotide
tccatgacgttcctgacgtt

SEQ ID NO: 3 - DNA, 24 bp, Artificial Sequence
  CpG oligonucleotide
tcgtcgttttgtcgttttgtcgtt

SEQ ID NO: 4 - DNA, 21 bp, Artificial Sequence
  CpG oligonucleotide
tcgtcgtttttcggtcgtttt

SEQ ID NO: 5 - DNA, 20 bp, Artificial Sequence
  CpG oligonucleotide
ataatcgacgttcaagcaag

SEQ ID NO: 6 - DNA, 13 bp, Artificial Sequence
  CpG oligonucleotide
ctcgagcgttctc

SEQ ID NO: 7 - DNA, 14 bp, Artificial Sequence
  CpG oligonucleotide
tctcgagcgttctc

SEQ ID NO: 8 - DNA, 16 bp, Artificial Sequence
  CpG oligonucleotide
actctggagcgttctc

SEQ ID NO: 9 - DNA, 12 bp, Artificial Sequence
  CpG oligonucleotide
tgcagcgttctc

SEQ ID NO: 10 - DNA, 12 bp, Artificial Sequence
  CpG oligonucleotide
tcgaggcttctc

SEQ ID NO: 11 - DNA, 12 bp, Artificial Sequence
  CpG oligonucleotide
gtcggcgttgac

SEQ ID NO: 12 - DNA, 19 bp, Artificial Sequence
  CpG oligonucleotide
tcgactctcgagcgttctc

SEQ ID NO: 13 - DNA, 20 bp, Artificial Sequence
  CpG oligonucleotide
atcgactctcgagcgttctc

SEQ ID NO: 14 - DNA, 12 bp, Artificial Sequence
  CpG oligonucleotide
tcgagcgttctc

SEQ ID NO: 15 - DNA, 12 bp, Artificial Sequence
  CpG oligonucleotide
gtcggcgtcgac

SEQ ID NO: 16 - DNA, 12 bp, Artificial Sequence
  CpG oligonucleotide
gtcgacgttgac

SEQ ID NO: 17 - DNA, 16 bp, Artificial Sequence
  CpG oligonucleotide
actctcgagggttctc

SEQ ID NO: 18 - DNA, 16 bp, Artificial Sequence
  CpG oligonucleotide
actctcgagcgttctc

SEQ ID NO: 19 - DNA, 12 bp, Artificial Sequence
  CpG oligonucleotide
gtcgtcgatgac

SEQ ID NO: 20 - DNA, 12 bp, Artificial Sequence
  CpG oligonucleotide
gtcgacgctgac

SEQ ID NO: 21 - DNA, 12 bp, Artificial Sequence
  CpG oligonucleotide
gtcgacgtcgac

SEQ ID NO: 22 - DNA, 12 bp, Artificial Sequence
  CpG oligonucleotide
gtcatcgatgca

SEQ ID NO: 23 - DNA, 12 bp, Artificial Sequence
  CpG oligonucleotide
gtcagcgtcgac

SEQ ID NO: 24 - DNA, 11 bp, Artificial Sequence
  CpG oligonucleotide
tcgagcgttct

SEQ ID NO: 25 - DNA, 16 bp, Artificial Sequence
  CpG oligonucleotide
actctggagcgttctc

SEQ ID NO: 26 - DNA, 16 bp, Artificial Sequence
  CpG oligonucleotide
actctcgagggttctc

SEQ ID NO: 27 - DNA, 16 bp, Artificial Sequence
  CpG oligonucleotide
actctcgagcgttcta

SEQ ID NO: 28 - DNA, 16 bp, Artificial Sequence
  CpG oligonucleotide
catctcgagcgttctc

SEQ ID NO: 29 - DNA, 14 bp, Artificial Sequence
  CpG oligonucleotide
actctttcgttctc

SEQ ID NO: 30 - DNA, 11 bp, Artificial Sequence
  CpG oligonucleotide
tcgagcgttct

SEQ ID NO: 31 - DNA, 12 bp, Artificial Sequence
  CpG oligonucleotide
tcgttcgttctc

SEQ ID NO: 32 - DNA, 15 bp, Artificial Sequence
  CpG oligonucleotide
gctagacgttagcgt

SEQ ID NO: 33 - DNA, 12 bp, Artificial Sequence
  CpG oligonucleotide
tcgaggcttctc

SEQ ID NO: 34 - DNA, 20 bp, Artificial Sequence
  CpG oligonucleotide
tccatgagcttcctgagctt

SEQ ID NO: 35 - DNA, 15 bp, Artificial Sequence
  CpG oligonucleotide
tagagcttagcttgc

SEQ ID NO: 36 - DNA, 12 bp, Artificial Sequence
  CpG oligonucleotide
ttgagtgttctc

SEQ ID NO: 37 - DNA, 30 bp, Artificial Sequence
  CpG oligonucleotide
  misc_feature (1)..(3): n is any nucleotide
  misc_feature (10)..(12): n is any nucleotide
  misc_feature (13)..(22): n is any nucleotide or no nucleotide
  misc_feature (27)..(30): n is G or no nucleotide
nnnrycgrynnnnnnnnnnnnnggggnnnn

SEQ ID NO: 38 - DNA, 20 bp, Artificial Sequence
  CpG oligonucleotide
  misc_feature (1)..(2): N can be any nucleotide, or not present
nntgcatcgatgcagggggg

SEQ ID NO: 39 - DNA, 20 bp, Artificial Sequence
  CpG oligonucleotide
  misc_feature (1)..(2): N can be any nucleotide, or not present
nntgcaccggtgcagggggg

SEQ ID NO: 40 - DNA, 20 bp, Artificial Sequence
  CpG oligonucleotide
  misc_feature (1)..(2): N can be any nucleotide, or not present
nntgcgtcgacgcagggggg

SEQ ID NO: 41 - DNA, 20 bp, Artificial Sequence
  CpG oligonucleotide
  misc_feature (1)..(2): N can be any nucleotide, or not present
nntgcgtcgatgcagggggg

SEQ ID NO: 42 - DNA, 20 bp, Artificial Sequence
  CpG oligonucleotide
  misc_feature (1)..(2): N can be any nucleotide, or not present
nntgcgccggcgcagggggg

SEQ ID NO: 43 - DNA, 20 bp, Artificial Sequence
  CpG oligonucleotide
  misc_feature (1)..(2): N can be any nucleotide, or not present
nntgcgccgatgcagggggg

SEQ ID NO: 44 - DNA, 20 bp, Artificial Sequence
  CpG oligonucleotide
  misc_feature (1)..(2): N can be any nucleotide, or not present
nntgcatcgacgcagggggg

SEQ ID NO: 45 - DNA, 20 bp, Artificial Sequence
  CpG oligonucleotide
  misc_feature (1)..(2): N can be any nucleotide, or not present
nntgcgtcggtgcagggggg

SEQ ID NO: 46 - DNA, 20 bp, Artificial Sequence
  CpG oligonucleotide
ggtgcatcgatgcagggggg

SEQ ID NO: 47 - DNA, 20 bp, Artificial Sequence
  CpG oligonucleotide
ggtgcatcgatgcagggggg

SEQ ID NO: 48 - DNA, 20 bp, Artificial Sequence
  CpG oligonucleotide
ggtgcaccggtgcagggggg

SEQ ID NO: 49 - DNA, 20 bp, Artificial Sequence
  CpG oligonucleotide
ggtgcatcgatgcagggggg

SEQ ID NO: 50 - DNA, 20 bp, Artificial Sequence
  CpG oligonucleotide
ggtgcgtcgatgcagggggg

SEQ ID NO: 51 - DNA, 20 bp, Artificial Sequence
  CpG oligonucleotide
ggtgtgtcgatgcagggggg

SEQ ID NO: 52 - DNA, 18 bp, Artificial Sequence
  CpG oligonucleotide
tgcatcgatgcagggggg

SEQ ID NO: 53 - DNA, 20 bp, Artificial Sequence
  CpG oligonucleotide
ggtgcatcgatacagggggg

SEQ ID NO: 54 - DNA, 20 bp, Artificial Sequence
  CpG oligonucleotide
ggtgcatcgatgcagggggg

SEQ ID NO: 55 - DNA, 20 bp, Artificial Sequence
  CpG oligonucleotide
ggtgcatcgttgcagggggg

SEQ ID NO: 56 - DNA, 20 bp, Artificial Sequence
  CpG oligonucleotide
ggtgcgtcgacgcagggggg

SEQ ID NO: 57 - DNA, 20 bp, Artificial Sequence
  CpG oligonucleotide
ggtcgatcgatgcacggggg

SEQ ID NO: 58 - DNA, 20 bp, Artificial Sequence
  CpG oligonucleotide
ggtgcatcgatgcagggggg

SEQ ID NO: 59 - DNA, 20 bp, Artificial Sequence
  CpG oligonucleotide
ggtgcatcgacgcagggggg

SEQ ID NO: 60 - DNA, 20 bp, Artificial Sequence
  CpG oligonucleotide
ggtgcatcgataggcggggg

SEQ ID NO: 61 - DNA, 20 bp, Artificial Sequence
  CpG oligonucleotide
ggtgcaccgatgcagggggg

SEQ ID NO: 62 - DNA, 20 bp, Artificial Sequence
  CpG oligonucleotide
cctgcatcgatgcagggggg

SEQ ID NO: 63 - DNA, 20 bp, Artificial Sequence
  CpG oligonucleotide
ggtatatcgatatagggggg

SEQ ID NO: 64 - DNA, 20 bp, Artificial Sequence
  CpG oligonucleotide
ggtggatcgatccagggggg

SEQ ID NO: 65 - DNA, 20 bp, Artificial Sequence
  CpG oligonucleotide
gggggacgatcgtcgggggg

SEQ ID NO: 66 - DNA, 25 bp, Artificial Sequence
  CpG oligonucleotide
tcgtcgtcgttcgaacgacgttgat

SEQ ID NO: 67 - DNA, 20 bp, Artificial Sequence
  CpG oligonucleotide
tccatgacgttcctgatgct

SEQ ID NO: 68 - DNA, 22 bp, Artificial Sequence
  CpG oligonucleotide
tcgtcgttttcggcgcgcgccg

SEQ ID NO: 69 - DNA, 21 bp, Artificial Sequence
  CpG oligonucleotide
tcgtcgaacgttcgagatgat

SEQ ID NO: 70 - protein, 15 aa, BK virus
TLYKKMEQDVKVAHQ

SEQ ID NO: 71 - protein, 15 aa, BK virus
GNLPLMRKAYLRKCK

SEQ ID NO: 72 - protein, 15 aa, BK virus
TFSRMKYNICMGKCI

SEQ ID NO: 73 - protein, 9 aa, JC virus
SITEVECFL

SEQ ID NO: 74 - protein, 9 aa, Human herpesvirus 4
QPRAPIRPI

SEQ ID NO: 75 - protein, 9 aa, Human cytomegalovirus
NLVPMVATV

SEQ ID NO: 76 - protein, 9 aa, Human papillomavirus type 16
YMLDLQPET

SEQ ID NO: 77 - protein, 9 aa, Influenza A virus
GILGFVFTL

SEQ ID NO: 78 - protein, 24 aa, Blastomyces dermatitidis
CELDNSHEDYNWNLWFKWCSGHGR

SEQ ID NO: 79 - protein, 24 aa, Blastomyces dermatitidis
TGHGKHFYDCDWDPSHGDYSWYLW

SEQ ID NO: 80 - protein, 24 aa, Blastomyces dermatitidis
DPSHGDYSWYLWDYLCGNGHHPYD

SEQ ID NO: 81 - protein, 24 aa, Blastomyces dermatitidis
DYLCGNGHHPYDCELDNSHEDYSW

SEQ ID NO: 82 - protein, 24 aa, Blastomyces dermatitidis
DPYNCDWDPYHEKYDWDLWNKWCN

SEQ ID NO: 83 - protein, 24 aa, Blastomyces dermatitidis
KYDWDLWNKWCNKDPYNCDWDPYH

SEQ ID NO: 84 - protein, 8 aa, Artificial Sequence
  Synthetic peptide
SIINFEKL

SEQ ID NO: 85 - protein, 9 aa, Artificial Sequence
  Synthetic peptide
CSIINFEKL

SEQ ID NO: 86 - protein, 9 aa, Artificial Sequence
  Synthetic peptide
ASMTNMELM

SEQ ID NO: 87 - protein, 20 aa, Human papillomavirus type 16
GQAEPDRAHYNIVTFCCKCD

SEQ ID NO: 88 - protein, 10 aa, Human papillomavirus type 16
CRAHYNIVTF